(19) United States
(12) Patent Application Publication
(10) Pub. No.: US 2004/0138160 A1
(43) Pub. Date: Jul. 15, 2004

(54) Preventive/therapeutic method for cancer

(76) Inventors: Kenichiro Naito, Mino-shi (JP); Shuichi Furuya, Kobe-shi (JP); Akihiro Tasaka, Suita-shi (JP); Toshikazu Ban, Ikoma-shi (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/475,990

(22) PCT Filed: Apr. 26, 2002
(86) PCT No.: PCT/JP02/04217

(30) Foreign Application Priority Data
Apr. 27, 2001 (JP) 2001-131613
Mar. 22, 2002 (JP) 2002-082019

(51) Int. Cl. A61K 48/00; A61K 39/395

(52) U.S. Cl. 514/44; 424/155.1

(57) ABSTRACT

This invention provides a prophylactic or therapeutic method for cancer.

A prophylactic or therapeutic method for cancer, which is characterized by selectively inhibiting ErbB-2 (HER2) to block information signals of multimers of the epithelial growth factor receptor family.

### TECHNICAL FIELD TO WHICH THE INVENTION BELONGS

The present invention relates to a method of preventing or treating cancer.

### BACKGROUND ART

The NEU oncogene found from rat neuroblastoma induced by a chemical carcinogen has been found to encode protein belonging to the EGF receptor family, indicating the relationship with the EGF receptor family. Thereafter, NEU human homolog was isolated and named ERBB2 or HER2 in view of similarity to the EGF receptor (ERBB). It has been reported that HER2 expresses in breast cancer, prostate cancer, lung cancer, gastric cancer and the like, and HER2 is considered to be involved in the growth of these cancers.

For example, it has been reported that, in prostate cancer, about 25% tests HER2 expression positive when it is a primary cancer, and the proportion increases as the cancer progresses (Journal of the National Cancer Institute, Vol. 92, No. 23, pp 1918-1925 (2000)).

Since HER2 scarcely expresses in tissues generally, a HER2 selective therapeutic agent becomes cancer selective, reduces toxicity and makes a therapeutic agent associated with extremely few side effects. Vastly different from conventional cancer chemotherapeutic agents, this leads to the provision of an extremely safe and highly versatile treatment method. As such HER2 selective therapeutic agent, for example, an inhibitor of tyrosine kinase (phosphorylated enzyme), which has high selectivity to HER2, and the like can be mentioned.

The problems to be overcome when treating cancer include (1) attenuation of treatment effect due to acquired drug tolerance of cancer tissue and (2) cancer metastasis.

(1) Various theories exist as regards the acquirement of the drug tolerance of cancer cells, and, for example, involvement of P-glycoprotein and the like has been suspected. A recent report has documented that the EGF receptor family members (EGF receptor, HER2, HER3 and the like) are mixed and express to promote drug tolerance (B.B.R.C., vol. 277, pp. 757-763 (2000)). Inhibitors of the EGF receptor family, such as a HER2-neutralizing antibody, are effective when HER2 alone is to be suppressed but, when the family members are present in mixture, an effective treatment by a sole inhibitor cannot be expected. In addition, tyrosine kinases (phosphorylated enzyme) having low selectivity may widely inhibit the EGF family, but may inhibit other tyrosine kinase (phosphorylated enzyme) as well, and use thereof at low toxicity is highly unlikely to be available.

(2) There are many texts regarding cancer metastasis and various theories are reported. Recently, the presence of a correlation between the frequency of the expression of HER3 (belonging to EGF receptor family) in cancer cells and the lymph node metastasis of said cancer cells has been reported (Eur. Arch. Otorhinolaryngol., Vol. 250, p 392 (1993)).

As a compound that inhibits receptor type tyrosine kinases, including HER2, Japanese Patent Unexamined Publication No. 60571/1999 discloses a compound represented by the formula:

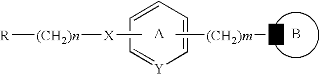

wherein R is an aromatic heterocyclic group which may be substituted; X is an oxygen atom, an optionally oxidized sulfur atom, —C(═O)— or —CH(OH)—; Y is CH or N; m is an integer from 0 to 10; n is an integer from 1 to 5; the cyclic group represented by the formula:

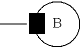

is an aromatic azole group which may be substituted; Ring A may be further substituted.

Thus, development of a superior method of preventing or treating cancer is desired, which can reduce side effects such as acquirement of drug tolerance of cancer tissue, cancer metastasis and the like.

### DISCLOSURE OF THE INVENTION

The present inventors have conducted intensive studies in an attempt to solve the above-mentioned problems and found that cancer can be unexpectedly prevented or treated efficiently by selectively inhibiting ErbB-2 (HER2) to block the information signal through the multimer of the epithelial growth factor receptor family, which resulted in the completion of the present invention.

Accordingly, the present invention relates to

[1] a method (A) for the prophylaxis or treatment of cancer, which comprises blocking an information signal through a multimer of an epithelial growth factor receptor family by selectively inhibiting ErbB-2 (HER2),

[2] the method of [1], wherein the information signal is blocked by inhibiting formation of a multimer between an ErbB-2 (HER2) and the epithelial growth factor receptor family,

[3] the method of [1] or [2], wherein the epithelial growth factor receptor family is EGFR (HER1), ErbB-2 (HER2), ErbB-3 (HER3) or ErbB-4 (HER4),

[4] the method of [1], wherein the multimer of the epithelial growth factor receptor family is a heterodimer between (1) ErbB-2 (HER2) and (2) an epithelial growth factor receptor family selected from EGFR (HER1), ErbB-3 (HER3) and ErbB-4 (HER4),

[5] the method of [1] to [4], wherein the cancer is a cancer including a cancer cell that tests HER2 positive by a HER2 diagnostic method such as Hercep Test™ and the like,

[6] the method of [1] to [5], wherein the ErbB-2 (HER2) is selectively inhibited by administering a HER2 tyrosine kinase inhibitor,

[7] the method of [1] to [5], wherein the ErbB-2 (HER2) is selectively inhibited by administering an inhibitor of an extracellular moiety of ErbB-2 (HER2),

[8] the method of [1] to [5], wherein the ErbB-2 (HER2) is selectively inhibited by administering a HER2 tyrosine kinase inhibitor and an anti-HER2 antibody in combination,

[9] the method of [8], wherein the anti-HER2 antibody is Trastuzumab,

[10] the method of [1] to [8], which is a prophylactic or therapeutic method of a drug resistant cancer,

[11] the method of [1] to [8], which is a prophylactic or therapeutic method of a hormone independent cancer,

[12] the method of [1] to [8], which is a prophylactic or therapeutic method of an anthracycline (doxorubicin and the like) resistant cancer,

[13] the method of [1] to [8], which is a prophylactic or therapeutic method of a taxon (Taxol, Taxotere and the like) resistant cancer,

[14] the method of [1] to [8], which is a prophylactic or therapeutic method of a platinum complex drug (cisplatin, carboplatin and the like) resistant cancer,

[15] the method of [1] to [8], which delays or prevents cancer's becoming hormone independent,

[16] a method (B) of inhibiting a cancer from acquiring drug resistance, which comprises selectively inhibiting ErbB-2 (HER2), thereby to block an information signal through the multimer of the epithelial growth factor receptor family,

[17] a method (C1) of inhibiting metastasis of a cancer, which comprises selectively inhibiting ErbB-2 (HER2), thereby to block an information signal through a multimer of the epithelial growth factor receptor family,

[18] a method (C2) of inhibiting metastasis of a cancer to a lymph node, which comprises selectively inhibiting ErbB-2 (HER2), thereby to block an information signal through a multimer of the epithelial growth factor receptor family,

[19] a method (D) for the prophylaxis or treatment of a cancer, which comprises inhibiting ErbB-2 (HER2) without causing downregulation of ErbB-2 (HER2) or going though an immune mechanism,

[20] a method (E) for regressing a cancer, which comprises selectively inhibiting ErbB-2 (HER2) to prevent involvement of the corresponding adaptor protein in ErbB-2 (HER2), thereby to block an information signal through a multimer of the epithelial growth factor receptor family,

[21] a method (F) of suppressing generation of an information signal through a HER2-HER3 dimer, which comprises inhibiting ErbB-2 (HER2) to prevent involvement of the corresponding adaptor protein in ErbB-3(HER3),

[22] a method (G) of delaying or prohibiting transition into a hormone independent cancer, which comprises administering a selective ErbB-2 (HER2) inhibitor to a patient with a hormone dependent cancer, and

[23] a method (H) for treating a cancer, which comprises administering a selective ErbB-2 (HER2) inhibitor to a patient with a hormone independent cancer to make cancer cells hormone-dependent, and administering a different anticancer agent and/or a hormone therapy agent to said patient, and the like.

Moreover, the present invention relates to

[24] the method according to [6] or [8], wherein the HER2 tyrosine kinase inhibitor is a compound of the formula:

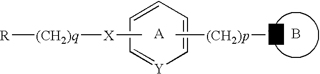

wherein R is an aromatic heterocyclic group which may be substituted; X is an oxygen atom, an optionally oxidized sulfur atom, —C(═O)— or —CH(OH)—; Y is CH or N; p is an integer from 0 to 10; q is an integer from 1 to 5; the group represented by the formula

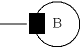

is an aromatic azole group which may be substituted; Ring A may be further substituted, or a salt or a prodrug thereof,

[25] the method according to [6] or [8], wherein HER2 tyrosine kinase inhibitor is a compound of the formula:

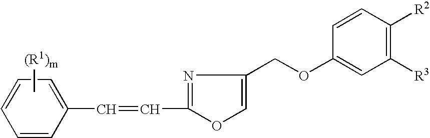

wherein m is 1 or 2;

R¹ is a halogen atom or an optionally halogenated C₁₋₂ alkyl group;

one of R² and R³ is a hydrogen atom and the other is a group represented by the formula:

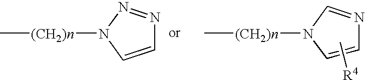

wherein n is 3 or 4; R⁴ is a C₁₋₄ alkyl group substituted by 1 or 2 hydroxy groups,

or a salt or a prodrug thereof,

[26] the method according to [24], wherein the cyclic group represented by the formula;

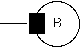

is pyrrolyl group, imidazolyl group, pyrazolyl group, triazolyl group, tetrazolyl group or benzimidazolyl group, each of which may be substituted with one or two groups selected from (i) alkyl group, (ii) aryl group, (iii) hydroxyalkyl group, (iv) carboxyl group, (v) alkoxycarbonyl group and (vi) carbamoyl group,

[27] The method according to [24], wherein p is an integer of 3 to 5,

[28] the method according to [24], wherein q is 1,

[29] the method according to [24], wherein X is oxygen atom,

[30] the method according to [24], wherein R is an optionally substituted oxazolyl group or an optionally substituted thiazolyl group,

[31] the method according to [24], wherein R is oxazolyl group, benzoxazolyl group or thiazolyl group, each of which may be substituted with one or two groups selected from (i) aryl group which may be substituted with one or two groups selected from hydroxyl group, alkoxy group, arylalkoxy group, alkyl group, cyano group, halogen atom and tetrazolyl group, (ii) alkyl group, (iii) hydroxyalkyl group, (iv) alkoxycarbonylalkyl group, (v) alkyl group substituted with one or two aryl groups, (vi) alkenyl group substituted with one or two aryl groups, (vii) cycloalkyl group, (viii) a partially saturated naphthyl group, (ix) thienyl group or furyl group, which may be substituted with one or two groups selected from hydroxy group, alkoxy group, arylalkoxy group, alkyl group, cyano group, allyl group and halogen atom, (x) benzofuranyl group and (xi) benzothienyl group,

[32] the method according to [24], wherein R is oxazolyl group, benzoxazolyl group or thiazolyl group, each of which may be substituted with one or two groups selected from (i) aryl group which may be substituted with one or two groups selected from hydroxyl group, alkoxy group, arylalkoxy group, alkyl group, cyano group, halogen atom and tetrazolyl group, (ii) alkyl group, (iii) hydroxyalkyl group, (iv) alkoxycarbonylalkyl group, (v) alkyl group substituted with one or two aryl groups, (vi) alkenyl group substituted with one or two aryl groups, (vii) cycloalkyl group, (viii) a partially saturated naphthyl group, (ix) thienyl group or furyl group, which may be substituted with one or two groups selected from hydroxy group, alkoxy group, arylalkoxy group, alkyl group, cyano group, allyl group and halogen atom, (x) benzofuranyl group and (xi) benzothienyl group,

X is oxygen atom,

p is an integer of 0 to 6,

q is 1,

the cyclic group represented by the formula;

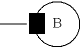

is pyrrolyl group, imidazolyl group, pyrazolyl group, triazolyl group, tetrazolyl group or benzimidazolyl group, which may be substituted with one or two groups selected from (i) alkyl group, (ii) aryl group, (iii) hydroxyalkyl group, (iv) carboxyl group, (v) alkoxycarbonyl group and (vi) carbamoyl group,

[33] the method according to [24], wherein R is oxazolyl group substituted with arylalkenyl or arylalkoxyaryl group,

X is oxygen atom,

p is 3 or 4,

q is 1,

the cyclic group represented by the formula;

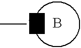

is imidazolyl group or triazolyl group,

the group represented by the formula;

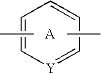

is 1,3-phenylene group or 1,4-phenylene group,

[34] the method according to [24], wherein. R is oxazolyl group substituted with thienyl or thiazolyl group substituted with thienyl,

X is oxygen atom,

p is 3 or 4,

q is 1 and

the cyclic group represented by the formula;

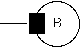

is imidazolyl group or triazolyl group,

the group represented by the formula;

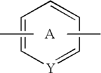

is 1,3-phenylene group or 1,4-phenylene group,

[35] the method according to [24], wherein R is benzoxazolyl group substituted with thienyl,

X is oxygen atom,

p is 3 or 4,

q is 1,

the cyclic group represented by the formula;

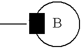

is imidazolyl group or triazolyl group,

the group represented by the formula;

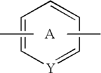

is 1,3-phenylene group or 1,4-phenylene group,

[36] the method according to [24], wherein the HER2 tyrosine kinase inhibitor is (i) 1-[4-[4-[2-[(E)-2-phenylethenyl]-4-oxazolylmethoxy]phenyl]butyl-1,2,4-triazole, (ii) 4-[4-[4-(1-imidazolyl)butyl]phenoxymethyl]-2-[(E)-2-phenylethenyl]oxazole, (iii) 4-[4-[3-(1-imidazolyl)propyl]phenoxymethyl]-2-[(E)-2-phenylethenyl]oxazole, (iv) 4-[3-[3-(1-imidazolyl)propyl]phenoxymethyl]-2-[(E)-2-phenylethenyl]oxazole, (v) 2-(4-benzyloxyphenyl)-4-[4-[4-[3-(1-imidazolyl)propyl]phenoxymethyl]oxazole, (vi) 4-[4-[3-(1-imidazolyl)propyl]phenoxymethyl]-2-(2-thienyl)oxazole, (vii) 4-[4-[3-(1-imidazolyl)propyl]phenoxymethyl]-2-(5-methyl-2-thienyl)oxazole, (viii) 2-(5-chloro-2-thienyl)-4-[4-[3-(1-imidazolyl)propyl]phenoxymethyl]oxazole, (ix) 4-[4-[3-(1-imidazolyl)propyl]phenoxymethyl]-2-(2-thienyl)thiazole, (x) 5-[4-[3-(1-imidazolyl)propyl]phenoxymethyl]-2-(2-thienyl)benzoxazole or a salt thereof or a prodrug thereof,

[37] the method according to [25], wherein R¹ is fluoro or trifluoromethyl,

[38] the method according to [25], wherein R² is a group represented by the formula:

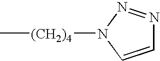

and R³ is a hydrogen atom; or

R² is a hydrogen atom and R³ is a group represented by the formula:

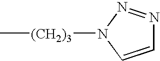

[39] the method according to [25], wherein R² is a group represented by the formula:

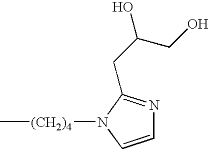

and R³ is a hydrogen atom,

[40] the method according to [25], wherein m is 1, R¹ is 4-trifluoromethyl, and R² is a group represented by the formula:

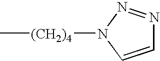

and R³ is a hydrogen atom, and

[41] the method according to [25], wherein the HER2 tyrosine kinase inhibitor is (i) 1-(4-{4-[(2-{(E)-2-[4-(trifluoromethyl)phenyl]ethenyl}-1,3-oxazol-4-yl)methoxy]phenyl}butyl)-1H-1,2,3-triazole, (ii) 1-(3-(3-[(2-{((E)-2-[4-(trifluoromethyl)phenyl]ethenyl}-1,3-oxazol-4-yl)methoxy]phenyl)propyl)-1H-1,2,3-triazole, or (iii) 3-(1-{4-[4-({2-[(E)-2-(2,4-difluorophenyl)ethenyl]-1,3-oxazol-4-yl}methoxy)phenyl]butyl}-1H-imidazol-2-yl)-1,2-propanediol, or

a salt thereof or a prodrug thereof.

### BRIEF DESCRIPTION OF THE DRAWINGS

FIG. 1 shows dose-dependent suppression of phosphorylation of compound B4 in BT-474 cell line that excessively expresses HER2, wherein the upper line shows the amount of phosphorylated HER2 and the lower line shows the amount of phosphorylated Akt.

FIG. 2 is a graph showing the cell death induction activity of compound B4 by inhibition of intracellular signal transduction, wherein the vertical axis shows the proportion (%) of the dead cell.

FIG. 3 shows HER2 phosphorylation inhibitory action of compound B4 in tumor.

FIG. 4 is a graph showing antitumor effect of compound B4, wherein the vertical axis shows relative tumor volume.

FIG. 5 shows the selectivity of compound B4 to HER2 expression cell.

### BEST MODE FOR EMBODYING THE INVENTION

The method (A) of the prophylaxis or treatment of cancer of the present invention characteristically inhibits ErbB-2 (HER2) selectively to block an information signal through a multimer of the epithelial growth factor receptor family.

As the epithelial growth factor receptor family, EGFR (HER1), ErbB-2 (HER2), ErbB-3 (HER3) and ErbB-4 (HER4) can be mentioned.

As a multimer between ErbB-2 (HER2) and the epithelial growth factor receptor family, a heterodimer between (1) ErbB-2 (HER2) and (2) an epithelial growth factor receptor family selected from EGFR (HER1), ErbB-3 (HER3) and ErbB-4 (HER4) is preferable.

For blocking the information signal, for example, inhibiting formation of a multimer between ErbB-2 (HER2) and an epithelial growth factor receptor family, and the like can be mentioned.

For selective inhibition of ErbB-2 (HER2), no particular limitation is imposed as long as it is a method capable of selectively inhibiting ErbB-2 (HER2). For example, an antibody (polyclonal antibody, monoclonal antibody) specific to ErbB-2 (HER2), a selective inhibitor of ErbB-2 (HER2), an expression suppressant of ErbB-2 (HER2), an antisense oligonucleotide to ErbB-2 (HER2) gene, a substance that inhibits promoter activity of ErbB-2 (HER2) gene and the like are preferably used.

More preferable methods for selectively inhibiting ErbB-2 (HER2) include,

(1) administering a HER2 tyrosine kinase inhibitor,

(2) administering an inhibitor of ErbB-2 (HER2) extracellular moiety,

(3) administering a HER2 tyrosine kinase inhibitor and an anti-HER2 antibody in combination, and the like.

As the HER2 tyrosine kinase inhibitor, for example, compound (I) to be mentioned later, a salt thereof, a prodrug thereof and the like are used.

As the inhibitor of ErbB-2 (HER2) extracellular moiety, HER2 receptor antagonist, an anti-HER 2 antibody and the like are used. As the anti-HER2 antibody, for example, Trastuzumab; Herceptin (trademarks) and the like are used.

Since the prophylactic or therapeutic method (A) of the present invention can selectively inhibit ErbB-2 (HER2) to block information signals of multimers of the epithelial growth factor receptor family, it is useful as a prophylactic or therapeutic method of cancer (e.g., breast cancer, prostate cancer, pancreatic cancer, gastric cancer, lung cancer, colon cancer, rectal cancer, esophagus cancer, duodenal cancer, cancer of the tongue, cancer of pharynx, cerebral cancer, neurilemoma, cancer of rectum, non-small cell lung cancer, small cell lung cancer, liver cancer, kidney cancer, cancer of the bile duct, cancer of the uterine body, cancer of the uterine cervix, ovarian cancer, bladder cancer, skin cancer, hemangioma, malignant lymphoma, malignant melanoma, thyroid carcinoma, bone tumors, hemangioma, vascular fibroma, retinoblastoma, penile cancer, solid cancer in childhood, Kaposi's sarcoma, Kaposi's sarcoma derived from AIDS, maxillary tumor, fibrous histiocytoma, leiomyosarcoma, rhabdomyosarcoma, leukemia and the like) in mammals (e.g., human, horse, bovine, dog, cat, rat, mouse, rabbit, pig, monkey etc.).

In addition, since the method of the present invention can specifically inhibit HER2, it can delay or inhibit cancer's becoming drug resistant or hormone independent.

Moreover, by performing the prophylactic or therapeutic method (A) of the present invention,

(1) acquisition of drug resistance of cancer can be inhibited (method (B) of the present invention),

(2) metastasis of cancer can be inhibited (method (C1) of the present invention), and

(3) metastasis of cancer to lymph node can be inhibited (method (C2) of the present invention).

The method (D) of the present invention is a method that prevents or treats cancer by inhibiting ErbB-2 (HER2) without causing downregulation of ErbB-2 (HER2) and without going through an immunomechanism.

By the downregulation of ErbB-2 (HER2) is meant uptake of HER2 molecule into the cell to decrease HER2 molecules on the cell membrane, due to stimulation by binding of HER2 molecule on cell membrane with a ligand, an antibody or any of the same kind and the like and activation of the proteolysis system of cells and the like.

By not going through immunomechanism is meant not causing any immunoresponse reaction in the living organisms.

As a means to not cause downregulation of ErbB-2 (HER2) and not go through an immunomechanism, inhibition of tyrosine kinase activity of ErbB-2 (HER2), a method of inhibiting formation of a dimer of ErbB-2 (HER2) and a HER family protein including the HER2 itself, by inhibition of a ligand binding etc., and the like can be mentioned.

According to the method (D) of the present invention, the aforementioned cancer can be prevented or treated efficiently.

The method (E) of the present invention is a method that achieves regression of cancer by selectively inhibiting ErbB-2 (HER2) to prevent involvement of the corresponding Adaptor protein in ErbB-2 (HER2), thereby blocking the information signal through the multimer of the epithelial growth factor receptor family.

The method for selectively inhibiting ErbB-2 (HER2) is as explained in the prophylactic or therapeutic method (A) of the present invention. Particularly, a method of administering compound (I) to be mentioned below or a salt thereof or a prodrug thereof and the like are used.

As the corresponding Adaptor protein, Grb2, shc, phosphatidylinositol 3 phosphate kinase (p85) and the like can be mentioned.

As the involvement of the corresponding Adaptor protein in ErbB-2 (HER2), binding with phosphotyrosin motif and the like can be mentioned.

In this way, binding of the corresponding Adaptor protein with ErbB-2 (HER2) can be inhibited by selectively inhibiting ErbB-2 (HER2), and information signals of multimers of the epithelial growth factor receptor family can be blocked.

Blocking of the information signals of multimers of the epithelial growth factor receptor family is as explained in the aforementioned prophylactic or therapeutic method (A) of the present invention.

According to the method (E) of the present invention, cancer can be regressed efficiently and the cancer can be effectively treated.

The method (F) of the present invention is a method that inhibits ErbB-2 (HER2) to prevent involvement of the corresponding Adaptor protein in ErbB-3 (HER), thereby suppressing generation of information signals through a HER2-HER3 dimer.

The means for inhibiting ErbB-2 (HER2) is not particularly limited as long as it is a method that can inhibit ErbB-2 (HER2). For example, an antibody against ErbB-2 (HER2) (polyclonal antibody, monoclonal antibody), ErbB-2 (HER2) inhibitor, ErbB-2 (HER2) expression suppressant, antisense oligonucleotide to ErbB-2 (HER2) gene, a substance that inhibits ErbB-2 (HER2) gene promoter activity and the like are preferably used.

More preferably, as a method for inhibiting ErbB-2 (HER2),

(1) administering a HER2 tyrosine kinase inhibitor,

(2) administering an inhibitor of ErbB-2 (HER2) extracellular moiety,

(3) administering a HER2 tyrosine kinase inhibitor and an anti-HER2 antibody in combination, and the like can be mentioned.

As a HER2 tyrosine kinase inhibitor, for example, compound (I) to be mentioned later, a salt thereof, a prodrug thereof and the like are used.

As the corresponding Adaptor protein, those similar to the aforementioned can be mentioned.

As the involvement of the corresponding Adaptor protein in ErbB-3 (HER3), phosphatidylinositol 3 phosphate kinase (p85), shc, Grb7 and the like can be mentioned.

In this way, inhibition of ErbB-2 (HER2) leads to the prevention of the involvement of the corresponding Adaptor protein in ErbB-3 (HER4), thereby suppressing generation of information signals through a HER2-HER3 dimer.

As the information signal through a HER2-HER3 dimer, cell growth signal, apoptosis resistant signal (survival signal), cell migration signal, angiogenesis inducing signal and the like can be mentioned.

According to method (F) of the present invention, generation of information signals through a HER2-HER3 dimer can be efficiently suppressed, and therefore, the aforementioned cancer can be effectively prevented or treated.

The method (G) of the present invention delays or prevents transition into hormone independent cancer by administering a selective ErbB-2 (HER2) inhibitor to patients with a hormone dependent cancer.

As the hormone dependent cancer, breast cancer, prostate cancer, ovarian cancer, cancer of uterine body, cervical cancer and the like can be mentioned.

As the selective ErbB-2 (HER2) inhibitor, for example, an antibody specific to ErbB-2 (HER2) (polyclonal antibody, monoclonal antibody), a selective inhibitor of ErbB-2 (HER2), expression suppressant of ErbB-2 (HER2), an antisense oligonucleotide to ErbB-2 (HER2) gene, a substance inhibiting promoter activity of ErbB-2 (HER2) gene and the like are used. Particularly, compound (I) to be mentioned later, a salt thereof, a prodrug thereof and the like are preferable.

According to method (G) of the present invention, transition from hormone dependent cancer to hormone independent cancer can be delayed or prevented. Thus, the aforementioned hormone dependent cancers can be effectively prevented or treated.

The method (H) of the present invention is a therapeutic method of cancer which characteristically comprises administration of a selective ErbB-2 (HER2) inhibitor to patients with hormone independent cancer to make the cancer cells hormone-dependent, after which other anticancer agent and/or hormone therapy agent are/is administered to the patients.

As the hormone independent cancer, those similar to the aforementioned can be mentioned.

As the selective ErbB-2-(HER2) inhibitor, those similar to the inhibitors used for method (G) of the present invention are used, and compound (I) to be mentioned later, a salt thereof, a prodrug thereof and the like are particularly preferable.

As other anticancer agents, various anticancer agents other than selective ErbB-2 (HER2) inhibitors are used. Specifically, anticancer agents to be mentioned later (e.g., chemical therapy agents, immunotherapy agents, pharmaceutical agent inhibiting action of cell growth factor and its receptor) and the like are used.

As the hormone therapy agents, those to be mentioned later and the like are used.

The dose of the anticancer agent is the same as that for compound (I) to be mentioned later, a salt thereof or a prodrug thereof.

According to method (H) of the present invention, since cancer cells can be once made hormone-dependent by the administration of a selective ErbB-2 (HER2) inhibitor, further subsequent administration of other anticancer agents and/or hormone therapy agents affords effective prophylaxis or treatment of the aforementioned hormone independent cancers.

As a method for assaying a receptor belonging to the epithelial growth factor receptor family, for example, gene diagnosis, diagnosis by antibody and the like are used.

While the gene diagnosis includes DNA diagnosis and mRNA diagnosis, DNA or RNA encoding the receptor belonging to the forementioned epithelial growth factor receptor family can be used as a probe in both cases.

The aforementioned probe may be any of DNA derived genomic DNAs, genomic DNA libraries, cDNAs derived from various cells/tissues, cDNA libraries derived from various cells/tissues, RNAs prepared from them, synthetic DNAs and synthetic RNAs. The vector to be used for the library may be any of bacteriophage, plasmid, cosmid, phagemid, and the like. It is also possible to prepare a total RNA or mRNA fraction from a cell/tissue and directly amplify same by Reverse Transcriptase Polymerase Chain Reaction (hereinafter to be abbreviated as RT-PCR method).

As the probe of the receptor belonging to the aforementioned epithelial growth factor receptor family, a DNA encoding HER2 (Nature, vol. 319, pp. 230-234 (1986)), a DNA encoding HER3 (Proceedings of the National Academy of Sciences of the United States of America, vol. 86, pp. 9193-9197 (1989)), a DNA encoding HER4 (Proceedings of the National Academy of Sciences of the United States of America, vol. 90, pp. 1746-1750 (1993)) and the like are specifically used.

Since the probe of the receptor belonging to the aforementioned epithelial growth factor receptor family can detect abnormality of DNA or mRNA encoding the receptor (gene abnormality) expressed in cancer cells, genetic diagnosis can be made, such as deletion, amplification, recombination (gene fusion), damage, mutation, lower expression, excessive expression and the like of said DNA or mRNA.

The genetic diagnosis can be performed by, for example, southern hybridization, FISH (fluorecence in situ hybridization), northern hybridization, PCR-SSCP method (Genomics, vol. 5, pp. 874-879 (1989); Proceedings of the National Academy of Sciences of the United States of America, vol. 86, pp. 2766-2770 (1989)), DNA chip method, DNA array method and the like, which are known per se.

The diagnosis using an antibody can be performed using an antibody to the aforementioned epidermal growth factor receptor. This antibody may be any of polyclonal antibody and monoclonal antibodies, as long as the antibody can recognize the aforementioned epidermal growth factor receptor. Since the antibody can recognize amino acid sequence, protein modification, steric structure and the like, for example, an antibody capable of recognizing changes in the protein modification state of this growth factor receptor, such as phosphorylation, acetylation, glycosylation and the like, as well as structural changes of the growth factor receptor without protein modification, such as amino acid mutation, steric structure and the like, and the like can be used.

As the antibody, a known antibody (e.g., trastuzumab which is an anti-HER2 antibody) can be used, or can be produced according to a method known per se for the production of antibody or antiserum using the aforementioned epidermal growth factor receptor as an antigen.

For diagnosis using an antibody, for example, western blotting, immunohistological staining, ELIZA and the like, which are known per se, can be used.

[Production of a Monoclonal Antibody]

(a) Establishment of Monoclonal Antibody-Producing Cells

The growth factor is administered with or without carrier and diluent to sites by which antibody production is induced in mammals. To increase the productivity of antibodies, Freund's complete or incomplete adjuvant-may be administered. Usually, the administration is performed every 2-6 weeks, 2-10 times in total. The mammal used includes monkeys, rabbits, dogs, guinea pigs, mice, rats, sheep, goats and chickens. Among them, mice and rats are preferably used.

In production of monoclonal antibody-producing cells, animals in which antibody titer are observed are selected from warm-blooded animals immunized with antigen such as mice, and the spleen or lymph nodes are excised 2-5 days after the final immunization. Monoclonal antibody-producing hybridomas can be produced by fusing the antibody-producing cells contained in the excised organ with myeloma cells. Antibody titer in an antiserum can be measured by, for example, reacting the labeled growth factor described below with the antiserum, followed by measurement of the activity of the label bound to the antibodies. The fusion manipulation can be performed according to known methods such as the method by Köhler and Milstein [Nature Vol. 256, 495 (1975)]. As the fusion-promoting agent, for example, polyethyleneglycol (PEG) and Sendai virus are used, and PEG is preferred.

For myeloma cells, for example, NS-1, P3U1, SP2/0 and the like are included, and P3U1 is preferably used. The preferable ratio of the number of antibody-producing cells (spleen cells) to that of myeloma cells is about 1:1-20:1. PEG (preferably PEG1000-PEG6000) is added at the concentration of about 10-80% and the cells are incubated at about 20-40° C., preferably about 30-37° C., for about 1-10 minutes, then, an efficient cell fusion can be performed.

Various methods can be used for screening monoclonal antibody-producing hybridomas. For example, culture supernatant of hybridoma is added to the growth factor adsorbed on a solid phase (e.g. microplate) directly or with carrier, and anti-immunoglobulin antibody labeled with radioactive substance, enzyme or the like (when the cells used for cell fusion are mouse cells, anti-mouse immunoglobulin antibody is used) or protein A is added, then the monoclonal antibody bound to the solid phase is detected. In other method, culture supernatant of hybridoma is added to anti-immunoglobulin antibody or protein A adsorbed on a solid phase, and the growth factor labeled with radioactive substance, enzyme or the like is added, then the monoclonal antibody bound to the solid phase is detected.

Monoclonal antibodies can be selected by publicly known methods or its modified methods. Usually, medium for animal cells supplemented with HAT (hypoxanthine, aminopterin, thymidine) can be used. Any medium in which hybridoma can grow is used for selection and clonal growth. For example, RPMI 1640 medium containing 1-20%, preferably 10-20% fetal calf serum, GIT medium (Wako Pure Chemical Industries, Ltd.) containing 1-10% fetal calf serum, serum-free medium for hybridoma culture (SFM-101, Nissui Pharmaceutical Co., Ltd.) and the like can be used. The temperature for culture is usually 20-40° C., preferably about 37° C. The duration of culture is usually 5 days to 3 weeks, preferably 1-2 weeks. Usually, the cells can be cultured under 5% CO₂ gas. The antibody titer in culture supernatant of hybridoma can be measured by the same procedure as that for antibody titer in antiserum described above.

(b) Purification of Monoclonal Antibody

The monoclonal antibodies can be separated and purified in the same manner as in general separation and purification of polyclonal antibodies by the methods for separation and purification of the immunoglobulins [e.g. salting out, alcohol precipitation, isoelectric precipitation, electrophoresis, adsorption-desorption method using ion exchangers (e.g. DEAE), ultracentrifugation, gel filtration, specific purification methods in which only antibody is collected using antigen-bound solid phase or an active adsorbent such as, protein A and protein G, and the antibody is obtained by dissociating the binding].

[Production of Polyclonal Antibodies]

The polyclonal antibodies of this invention can be manufactured by publicly known methods or modified methods thereof. For example, a conjugate of an immunogen (growth factor) and a carrier protein is prepared, and mammals are immunized by the same method as described for production of monoclonal antibody, and the material containing the antibodies against the growth factor are collected from said immunized animals, and the antibodies are separated and purified.

Regarding the complex of immunogen and carrier protein for immunizing mammals, any kind of carrier protein can be used at any conjugation ratio of the carrier to the hapten if the antibody is efficiently produced against the hapten crosslinked to the carrier for immunization. For example, bovine serum albumin, bovine thyroglobulin, keyhole limpet hemocyanin or the like is coupled to hapten at a weight ratio of carrier to hapten of about 0.1-20, preferably about 1-5.

Various condensation agents can be used for coupling of carrier to hapten. Active ester reagents containing glutaraldehyde, carbodiimide, maleimide active ester, thiol group, dithiopyridyl group or the like are used.

The condensation product is administered with or without carrier and diluent to the site at which antibody can be produced in warm-blooded animals. To increase the productivity of antibodies, Freund's complete or incomplete adjuvant may be administered when the condensation product is administered. Usually, the condensation product may be administered every once per about 2-6 weeks, about 3-10 times in total.

The polyclonal antibody can be collected from the blood, ascites and the like, preferably from the blood of warm-blooded animals immunized by the method described above.

The polyclonal antibody titer in antiserum can be measured by the same procedure as that for the serum antibody titer described above. The antibody can be separated and purified according to the same separation and purification method for immunoglobulin as that of the monoclonal antibody described above.

Since the antibodies can specifically recognize the aforementioned growth factor, the growth factor in a test solution can be quantified by the sandwich immunoassay and the like. For example,

(i) A test solution and a labeled growth factor are competitively reacted with an antibody, and the ratio of the labeled growth factor bound to the antibody is measured, or

(ii) a test solution, and antibody immobilized on a carrier, and a labeled antibody are simultaneously or sequentially reacted, and the activity of the label on the immobilized carrier is measured.

In (ii), it is preferable that one antibody recognizes the N-terminal region of the growth factor, and the other antibody reacts with the C-terminal region of the growth factor.

Using monoclonal antibody against the growth factor (hereinafter sometimes to be briefly referred to as monoclonal antibody), quantification of the growth factor, and also detection of said growth factor by tissue staining or the like can be performed. For these purposes, whole antibody molecules may be used, and F(ab′)₂, Fab′, and Fab fractions of the antibody molecule may also be used. Quantification methods using antibodies against the growth factor are not restricted. Any measurement method can be used in which the amount of antibody, antigen, or antibody-antigen complex corresponding to the amount of antigen (e.g. the amount of the protein of the growth factor) in the test solution is detected by a chemical or physical technique and the antigen amount is calculated from a standard curve prepared from standard solutions containing known amount of the antigen. For example, nephrometry, competitive method, immunometric method, and sandwich method are appropriately used, and the sandwich method described below is most preferable in regard to sensitivity and specificity.

For the labeling agent for the measurement methods using labeled substances, for example, radioisotopes, enzymes, fluorescent substances, and luminescent substances are used. For the radioisotope, for example, [¹²⁵I], [¹³¹I], [³H], [¹⁴C] and the like are preferred. As the enzyme described above, stable enzymes with high specific activity are preferred, for example, β-galactosidase, β-glucosidase, alkaline phsophatase, peroxidase, malate dehydrogenase and the like are used. For the fluorescent substance, for example, fluorescamine, fluorescein isothiocyanate and the like are used. For the luminescent substance, for example, luminol, luminol derivatives, luciferin, lucigenin and the like are used. The biotin-avidin system may be used for the binding of labeling agents to antibody or antigen.

For immobilization of antigen or antibody, physical adsorption may be used, and chemical coupling methods usually used for immobilization or fixing proteins, enzymes and the like may also be used. As the carrier, for example, insoluble polysaccharides such as agarose, dextran, cellulose, synthetic resin such as polystyrene, polyacrylamide and silicon, glass, and the like are used.

In the sandwich method, immobilized monoclonal antibody is reacted with test solution (primary reaction), then, with labeled monoclonal antibody (secondary reaction), and the amount of protein of the growth factor in the test solution can be quantified by measuring the activity of the label on the insoluble carrier. The order of the primary and secondary reactions may be reversed, and the reactions may also be performed simultaneously or sequentially. The labeling agents and the methods for immobilization can follow those described above.

In the immunoassay by the sandwich method, the antibody used for immobilized and labeled antibodies is not necessarily one species, and a mixture of two or more species of antibody may be used to increase the measurement sensitivity.

In the methods for measuring the growth factor by the sandwich method, for the monoclonal antibodies used in the primary and secondary reactions, antibodies that bind to different sites of the growth factor and the like are preferred. Thus, the antibodies for the primary and secondary reactions are, for example, when antibody used in the secondary reaction recognizes the C-terminal region of the growth factor, it is preferable to use antibody recognizing the region other than the C-terminal region for the primary reaction, for example, antibody recognizing the N-terminal region.

Monoclonal antibodies can be used for measurement systems other than the sandwich method, for example, competitive method, immunometric method, nephrometry and the like. In the competitive method, antigen in test solution and the labeled antigen are competitively reacted with antibody, and (F): the non-reacted labeled antigen and (B): the labeled antigen bound to the antibody are separated (B/F separation). The amount of the label in B or F is measured, and the amount of the antigen in the test solution is quantified. For the reaction method, a liquid phase method using a soluble antibody, polyethylene glycol for B/F separation, and the secondary antibody against the soluble antibody, or an immobilized method using immobilized antibody as the first antibody or soluble antibody as the first antibody and an immobilized antibody as the secondary antibody is used.

In the immunometric method, antigen in test solution and immobilized antigen are competitively reacted with a specified amount of labeled antibody, then the solid phase and liquid phase are separated, or antigen in test solution and an excess amount of labeled antibody are reacted, then immobilized antigen is added to bind to the non-reacted labeled antibody to the solid phase, and the solid phase and liquid phases are separated. Then, the amount of the label in either phase is measured to quantify the antigen in the test solution.

In the nephrometry, insoluble precipitate produced after antigen-antibody reaction in gel or in solution is quantified. When the amount of antigen in the test solution is small and only a small amount of precipitate is obtained, for example, laser nephrometry using laser scattering is appropriately used.

For applying these immunological measurement methods to the quantification methods for this invention, no specific conditions, procedures or the like are necessary. Systems for measuring the epidermal growth factor are constructed by adding the usual technical consideration in the art to the conventional conditions and procedures. The details of these general technical means can be referred to reviews and texts. For example, Irie, H. ed. ‘Radioimmunoassay’ (Kodansha, 1974); Irie, H. ed. ‘Sequel to the Radioimmunoassay’ (Kodansha, 1979); Ishikawa, E. et al. ed. ‘Immunoenzyme assay’ 25′ (Igakushoin, 1978); Ishikawa, E. et al. ed. ‘Immunoenzyme assay’ (2nd ed.) (Igakushoin, 1982); Ishikawa, E. et al. ed. ‘Immunoenzyme assay’ (3rd ed.) (Igakushoin, 1987); Methods in ENZYMOLOGY [Vol. 70 (Immunochemical Techniques (Part A)), Vol. 73 (Immunochemical Techniques (Part B)), Vol. 74 (Immunochemical Techniques (Part C)), Vol. 84 (Immunochemical Techniques (Part D: Selected Immunoassays)), Vol. 92 (Immunochemical Techniques (Part E: Monoclonal Antibodies and General Immunoassay Methods)), Vol. 121 (Immunochemical Techniques (Part I: Hybridoma Technology and Monoclonal Antibodies)) (Academic Press Publishing)]; and the like can be referred.

By using the antibodies as mentioned above, the epidermal growth factor can be quantified with high sensitivity.

As a HER2 tyrosine kinase inhibitor, compound (I) represented by the formula

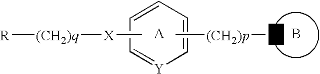

wherein R is an aromatic heterocyclic group which may be substituted; X is an oxygen atom, an optionally oxidized sulfur atom, —C(═O)— or —CH(OH)—; Y is CH or N; p is an integer from 0 to 10; q is an integer from 1 to 5; the group represented by the formula

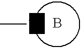

is an aromatic azole group which may be substituted; Ring A may be further substituted, or a salt thereof or a prodrug thereof and the like is used.

In the above-mentioned the formula (I), as the heterocyclic group in the optionally substituted aromatic heterocyclic group shown by R, for example, (1) 5- or 6-membered aromatic monocyclic heterocyclic group containing as the ring-forming atoms, besides carbon atoms, 1 to 4 atoms selected from nitrogen atom, oxygen atom and sulfur atom, and (2) aromatic condensed heterocyclic group formed by condensation of (i) 5- or 6-membered aromatic monocyclic heterocyclic group containing, as the ring-forming atoms, besides carbon atoms, 1 to 4 atoms selected from nitrogen atom, oxygen atom and sulfur atom with (ii) 5- or 6-membered aromatic or non-aromatic heterocyclic group containing, as the ring-forming atoms, besides carbon atoms, 1 or 2 nitrogen atoms, benzene ring or 5-membered aromatic or non-aromatic heterocyclic group containing, as the ring-forming atoms, besides carbon atoms, one sulfur atom.

Specific examples of these aromatic heterocyclic groups include pyridyl (e.g. 2-pyridyl, 3-pyridyl, 4-pyridyl), pyrimidinyl (e.g. 2-pyrimidinyl, 5-pyrimidinyl, 6-pyrimidinyl), pyridazinyl (e.g. 3-pyridazinyl, 4-pyridazinyl), pyrazinyl (e.g. 2-pyrazinyl), pyrrolyl (e.g. 1-pyrrolyl, 2-pyrrolyl), imidazolyl (e.g. 1-imidazolyl, 2-imidazolyl, 4-imidazolyl, 5-imidazolyl), pyrazolyl (e.g. 1-pyrazolyl, 3-pyrazolyl, 4-pyrazolyl), isoxazolyl, isothiazolyl, thiazolyl (e.g. 2-thiazolyl, 4-thiazolyl, 5-thiazolyl), oxazolyl (e.g. 2-oxazolyl, 4-oxazolyl, 5-oxazolyl), oxadiazolyl (e.g. 1,2,4-oxadiazolyl such as 1,2,4-oxadiazol-5-yl, 1,2,3-oxadiazolyl, 1,3,4-oxadiazolyl), thiadiazolyl (e.g. 1,2,3-thiadiazolyl, 1,2,4-thiadiazolyl, 1,3,4-thiadiazolyl), triazolyl (e.g. 1,2,4-triazolyl such as 1,2,4-triazol-1-yl, 1,2,4-triazol-5-yl, 1,2,3-triazolyl such as 1,2,3-triazol-1-yl, 1,2,3-triazol-2-yl, 1,2,3-triazol-4-yl), tetrazolyl (e.g. tetrazol-1-yl, tetrazol-5-yl), benzimidazolyl (e.g. benzimidazol-1-yl, benzimidazol-2-yl), indolyl (e.g. indol-1-yl, indol-3-yl), indazolyl (e.g. 1H-indazol-1-yl, 1H-indazol-3-yl), pyrrolopyrazinyl (e.g. 1H-pyrrolo[2,3-b]pyrazinyl), pyrrolopyridyl (e.g. 1H-pyrrolo[2,3-b]pyridyl), imidazopyridyl (e.g. 1H-imidazo[4,5-b]pyridyl, 1H-imidazo[4,5-c]pyridyl), imidazopyrazinyl (e.g. 1H-imidazo[4,5-b]pyrazinyl), pyrrolopyridazinyl (e.g. pyrrolo[1,2-b]pyridazinyl), pyrazolopyridyl (e.g. pyrazolo[1,5-a]pyridyl), imidazopyridyl (e.g. imidazo[1,2-a]pyridyl, imidazo[1,5-a]pyridyl), imidazopyridazinyl (e.g. imidazo[1,2-b]pyridazinyl), imidazopyrimidinyl (e.g. imidazo[1,2-a]pyrimidinyl), furyl, thienyl, benzofuranyl, benzothienyl (e.g. benzo[b]thienyl), benzoxazolyl, benzthiazolyl, quinolyl, isoquinolyl and quinazolinyl. Preferable examples include 5-membered monocyclic aromatic azole group such as oxazolyl, thiazolyl, isoxazolyl, isothiazolyl, imidazolyl, triazolyl, oxadiazolyl and thiadiazolyl, aromatic condensed azole group formed by condensation with benzene ring such as benzoxazolyl and benzthiazolyl, and 6-membered monocyclic aromatic heterocyclic group such as pyridyl and pyrimidyl. Further preferable examples of the aromatic heterocyclic group include 5-membered monocyclic aromatic azole group such as oxazolyl group and thiazolyl group.

As the aromatic heterocyclic group represented by R and the aromatic azole group represented by the formula:

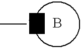

for example, (1) 5-membered aromatic monocyclic heterocyclic group containing, as the ring-forming atoms, besides carbon atoms, 1 to 4 nitrogen atoms and optionally containing one oxygen atom or one sulfur atom, and (2) aromatic condensed heterocyclic group formed by condensation of (i) 5-membered aromatic monocyclic heterocyclic group containing, as the ring-forming atoms, besides carbon atoms, 1 to 4 nitrogen atoms and optionally containing one oxygen atom or one sulfur atom, with (ii) 5- or 6-membered aromatic or non-aromatic heterocyclic group containing, as the ring-forming atoms, besides carbon atoms, one or two nitrogen atoms, benzene ring or 5-membered aromatic or non-aromatic heterocyclic group containing, as the ring-forming atoms, besides carbon atoms, one sulfur atom, can be mentioned.

Specific examples of the aromatic azole group include aromatic heterocyclic group such as pyrrolyl (e.g. 1-pyrrolyl), imidazolyl (e.g. 1-imidazolyl), pyrazolyl (e.g. 1-pyrazolyl), triazolyl (e.g. 1,2,4-triazol-1-yl, 1,2,3-triazol-1-yl), tetrazolyl (e.g. tetrazol-1-yl), benzimidazolyl (e.g. benzimidazol-1-yl), indolyl (e.g. indol-1-yl), indazolyl (e.g. 1H-indazol-1-yl), pyrrolopyrazinyl (e.g. 1H-pyrrolo[2,3-b]pyrazin-1-yl), pyrrolopyridyl (e.g. 1H-pyrrolo[2,3-b]pyridin-1-yl), imidazopyridyl (e.g. 1H-imidazo[4,5-b]pyridin-1-yl), and imidazopyrazinyl (e.g. 1H-imidazo[4,5-b]pyrazin-1-yl). These groups are bonded to —(CH₂)ₘ— through 10 the nitrogen atom contained as one of the ring-forming atoms.

Preferable examples of the aromatic azole group include imidazolyl group and triazolyl group.

The aromatic heterocyclic group represented by R and the aromatic azole group represented by the formula:

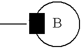

may have 1 to 3 (preferably one or two) substituents at any substitutable position. Examples of the substituents include aliphatic hydrocarbon group, alicyclic hydrocarbon group, aromatic hydrocarbon group, aliphatic hydrocarbon group substituted with aromatic hydrocarbon group, aliphatic hydrocarbon group substituted with alicyclic hydrocarbon group, aromatic heterocyclic group, non-aromatic heterocyclic group, aliphatic hydrocarbon group substituted with aromatic heterocyclic group, halogen atom, nitro group, cyano group, optionally substituted amino group, optionally substituted acyl group, optionally substituted hydroxyl group, optionally substituted thiol group, and optionally esterified and amidated carboxyl group. The aliphatic hydrocarbon group, alicyclic hydrocarbon group, aromatic hydrocarbon group, aliphatic hydrocarbon group substituted with aromatic hydrocarbon group, aliphatic hydrocarbon group substituted with alicyclic hydrocarbon group, aromatic heterocyclic group, non-aromatic heterocyclic group and aliphatic hydrocarbon group substituted with aromatic heterocyclic group may be further optionally substituted, respectively.

The ring A may optionally have, besides X and (CH₂)ₚ, 1 to 4 (preferably one or two) substituents at any substitutable position. As the substituents, mention is made of those exemplified as substituents which the substituents on the aromatic heterocyclic group represented by R may optionally have, as exemplified by aliphatic hydrocarbon group, alicyclic hydrocarbon group, aromatic hydrocarbon group, aliphatic hydrocarbon group substituted with aromatic hydrocarbon group, aliphatic hydrocarbon group substituted with alicyclic hydrocarbon group, aromatic heterocyclic group, non-aromatic heterocyclic group, aliphatic hydrocarbon group substituted with aromatic heterocyclic group, halogen atom, nitro group, cyano group, optionally substituted amino group, optionally substituted acyl group, optionally substituted hydroxyl group, optionally substituted thiol group, and optionally esterified or amidated carboxyl group. The aliphatic hydrocarbon group, alicyclic hydrocarbon group, aromatic hydrocarbon group, aliphatic hydrocarbon group substituted with aromatic hydrocarbon group, aliphatic hydrocarbon group substituted with alicyclic hydrocarbon group, aromatic heterocyclic group, non-aromatic heterocyclic group and aliphatic hydrocarbon group substituted with aromatic heterocyclic group mentioned above as substituents may optionally be further substituted.

As the aliphatic hydrocarbon group, mention is made of straight-chain or branched aliphatic hydrocarbon group having 1 to 15 carbon atoms, such as alkyl group, alkenyl group and alkynyl group.

Preferable examples of the alkyl group include C₁₋₁₀ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, nonyl, decyl, 1,1-dimethylbutyl, 2,2-dimethylbutyl, 3,3-dimethylbutyl and 2-ethylbutyl, more preferably C₁₋₆ alkyl group.

Preferable examples of the alkenyl group include C₂₋₁₀ alkenyl group such as vinyl (ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, more preferably C₂₋₆ alkenyl group.

Preferable examples of the alkynyl group include C₂₋₁₀ alkynyl group such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, more preferably C₂₋₆ alkynyl group.

Examples of the alicyclic hydrocarbon group include C₃₋₁₂ saturated or unsaturated alicyclic hydrocarbon group such as cycloalkyl group, cycloalkenyl group, cycloalkadienyl group or partially unsaturated condensed bicyclic hydrocarbon group.

Preferable examples of the cycloalkyl group include C₃₋₁₀ cycloalkyl group such as cyclopropyl, cyclobutyl, cyclopentyl, cyclohexyl, cycloheptyl and cyclooctyl; and C₆₁₀ bicycloalkyl group such as bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl and bicyclo[4.3.1]decyl.

Preferable examples of the cycloalkenyl group include C₅₋₁₀ cycloalkenyl group such as 2-cyclopenten-1-yl, 3-cyclopenten-1-yl, 2-cyclohexen-1-yl and 3-cyclohexen-1-yl.

Preferable examples of the cycloalkadienyl group include C₅₋₁₀ cycloalkadienyl group such as 2,4-cyclopentadien-1-yl, 2,4-cyclohexadien-1-yl and 2,5-cyclohexadien-1-yl.

Preferable examples of the partially unsaturated condensed bicyclic hydrocarbon group include C₉₋₁₂ group formed by condensation of benzene ring with alicyclic hydrocarbon, such as indanyl group, partially saturated naphthyl group (e.g. dihydronaphthyl group such as 3,4-dihydro-2-naphthyl; and tetrahydronaphthyl such as 1,2,3,4-tetrahydronaphthyl).

As the aromatic hydrocarbon group, mention is made of monocyclic or condensed polycyclic aromatic hydrocarbon group, preferably exemplified by C₆₋₁₄ aryl group such as phenyl, naphthyl, anthryl, phenanthryl acenaphthylenyl and 9-fluorenon-2-yl. Among them, monocyclic or condensed bicyclic aromatic hydrocarbon groups such as phenyl, 1-naphthyl and 2-naphthyl are preferable.

As the aliphatic hydrocarbon group substituted with aromatic hydrocarbon group, mention is made of, for example, aliphatic hydrocarbon group substituted with 1 to 3 (preferably 1 or 2) C₇₋₂₀ aromatic hydrocarbon groups. Preferable examples of such aliphatic hydrocarbon groups substituted with aromatic hydrocarbon groups as above include C₁₋₆ alkyl group substituted with 1 to 3 C₆₋₁₄ aryl groups (e.g. C₁₋₆ alkyl group substituted with 1 to 3 phenyl groups such as benzyl, 2-phenylethyl, 1,2-diphenylethyl and 2,2-diphenylethyl, C₁₋₆ alkyl group substituted with 1 to 3 naphthyl, and 9-fluorenyl-C₁₋₆ alkyl group) and C₂₋₆ alkenyl group substituted with 1 to 3 C₆₋₁₄ aryl groups (e.g. C₂₋₆ alkenyl group substituted with 1 to 3 phenyl groups, such as (E)-2-phenylethenyl, (Z)-2-phenylethenyl, 2,2-diphenylethenyl, 2-(2-napthyl)ethenyl and 4-phenyl-1,3-butadienyl, C₂₋₆ alkenyl group substituted with 1 to 3 naphthyl groups), and 9-fluorenylidenealkyl group.

As the aliphatic hydrocarbon group substituted with alicyclic hydrocarbon group, mention is made of the above-mentioned aliphatic hydrocarbon group substituted with 1 to 3 (preferably one or two) of the above-mentioned alicyclic hydrocarbon groups. Preferable examples of such aliphatic hydrocarbon group substituted with alicyclic hydrocarbon group include C₁₋₆ alkyl group substituted with 1 to 3 C₃₋₁₀ cycloalkyl groups, C₂₋₆ alkenyl group substituted with 1 to 3 C₃₋₁₀ cycloalkyl groups, C₁₋₆ alkyl group substituted with 1 to 3 C₅₋₁₀ cycloalkenyl groups, and C₂₋₆ alkenyl group substituted with 1 to 3 C₅₋₁₀ cycloalkenyl groups, such as cyclopropylmethyl, cyclopropylethyl, cyclobutylmethyl, cyclopentylmethyl, 2-cyclopentenylmethyl, 3-cyclopentenylmethyl, cyclohexylmethyl, 2-cyclohexenylmethyl, 3-cyclohexenylmethyl, cyclohexylethyl, cyclohexylpropyl, cycloheptylmethyl and cycloheptylethyl.

As preferable examples of the aromatic heterocyclic group, mention is made of the 5- or 6-membered aromatic monocyclic heterocyclic group containing, as ring-forming atoms, besides carbon atoms, 1 to 4 atoms selected from nitrogen atom, oxygen atom and sulfur atom, such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl and triazinyl; and the aromatic condensed heterocyclic group formed by condensation of (i) 5- or 6-membered aromatic heterocyclic group containing, as ring-forming atoms, besides carbon atoms, 1 to 4 atoms selected from nitrogen atom, oxygen atom and sulfur atom with (ii) 5- or 6-membered aromatic or non-aromatic heterocyclic group containing, as ring-forming atoms, besides carbon atoms, 1 or 2 nitrogen atoms, benzene ring or 5-membered aromatic or non-aromatic heterocyclic group containing, as ring-forming atoms, besides carbon atoms, one sulfur atom, such as benzofuranyl, isobenzofuranyl, benzo[b]thienyl, indolyl, isoindolyl, 1H-indazolyl, benzimidazolyl, benzoxazolyl, 1,2-benzisoxazolyl, benzothiazolyl, 1,2-benzisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, and 1,2,4-triazolo[4,3-b]pyridazinyl.

Preferable examples of the non-aromatic heterocyclic group include 3- to 7-membered non-aromatic heterocyclic group containing, as ring forming atoms, besides carbon atoms, 1 or 2 atoms selected from nitrogen atom, oxygen atom and sulfur atom, such as oxiranyl, azetidinyl, oxetanyl, thietanyl, pyrrolidinyl, tetrahydrofuryl, thiolanyl, piperidyl, tetrahydropyranyl, morpholinyl, thiomorpholinyl and piperazinyl.

As aliphatic hydrocarbon group substituted with aromatic heterocyclic group, mention is made of C₁₋₆ aliphatic hydrocarbon group substituted with 1 to 3 (preferably 1 or 2) of the above-mentioned aromatic heterocyclic groups (for example, C₁₋₆ alkyl group and C₂₋₆ alkenyl group). Preferable examples of the aliphatic hydrocarbon group substituted with aromatic heterocyclic group include C₁₋₆ alkyl group with 1 to 3 of, for example, furyl group, thienyl group, imidazolyl group or pyridyl group (e.g. (2-furyl)methyl, thienylmethyl and 2-(1-imidazolyl)ethyl), and C₂₋₆ alkenyl group substituted with 1 to 3 of furyl group, thienyl group, imidazolyl group or pyridyl group (e.g. 2-(2-furyl)ethenyl, 2-thienylethenyl).

As halogen atoms, mention is made of, for example, fluorine, chlorine, bromine and iodine, especially fluorine and chlorine being preferable.

As the optionally substituted amino group, mention is made of amino group optionally mono- or di-substituted with, for example, C₁₋₁₀ alkyl group, C₃₋₁₀ cycloalkyl group, C₂₋₁₀ alkenyl group, C₅₋₁₀ cycloalkenyl group, C₁₋₁₀ acyl group or C₆₁₂ aromatic hydrocarbon group (e.g. methylamino, dimethylamino, ethylamino, diethylamino, dibutylamino, diallylamino, cyclohexylamino, acetylamino, propionylamino, benzoylamino, phenylamino and N-methyl-N-phenylamino) and 4- to 6-membered cyclic amino group (e.g. 1-azetidinyl, 1-pyrrolidinyl, piperidino, morpholino and 1-piperazinyl).

The said 4- to 6-membered cyclic amino group may optionally be further substituted with (1) C₁₋₆ alkyl group, (2) C₆₋₁₄ aryl group optionally substituted with halogen, C₁₋₆ alkoxy group or trifluoromethyl (e.g. phenyl and naphthyl), (3)-5- or 6-membered heterocyclic group containing, as ring-forming atoms, besides carbon atoms, 1 or 2 nitrogen atoms (e.g. 2-pyridyl and pyrimidinyl) or (4) 6-membered cyclic amino group (e.g. piperidino and 1-piperazinyl).

As the acyl group of optionally substituted acyl group, mention is made of C₁₋₁₃ acyl group, more specifically, besides formyl, those formed by linkage of, for example, C₁₋₆ alkyl group, C₃₋₁₀ cycloalkyl group, C₂₋₆ alkenyl group, C₅₋₁₀ cycloalkenyl group, C₆₋₁₂ aromatic hydrocarbon group (e.g. phenyl and naphthyl) or aromatic heterocyclic ring (e.g. pyridyl) with carbonyl group, as exemplified by C₂₋₇ alkanoyl group (e.g. acetyl, propionyl, butyryl, isobutyryl, valeryl, isovaleryl, pivaloyl, hexanoyl, heptanoyl and octanoyl), C₃₋₁₀ cycloalkyl-carbonyl group (e.g. cyclobutanecarbonyl, cyclopentanecarbonyl, cyclohexanecarbonyl and cycloheptanecarbonyl), C₃₋₇ alkenoyl group (e.g. crotonoyl group), C₅₋₁₀ cycloalkenyl-carbonyl group (e.g. 2-cyclohexenecarbonyl), benzoyl group and nicotinoyl group.

As substituents in the optionally substituted acyl group, mention is made of, for example, C₁₋₃ alkyl group, C₁₋₃ alkoxy group, halogen (e.g. chlorine, fluorine and bromine), nitro group, hydroxyl group and amino group. The number of substituents ranges, for example, from 1 to 3.

Examples of the optionally substituted hydroxyl group include hydroxyl group, alkoxy group, cycloalkyloxy group, alkenyloxy group, cycloalkenyloxy group, aralkyloxy group, aryloxy group and acyloxy group.

Preferable examples of the alkoxy group include C-10 alkoxy group such as methoxy, ethoxy, propoxy, isopropoxy, butoxy, isobutoxy, sec-butoxy, tert-butoxy, pentyloxy, isopentyloxy, neopentyl, hexyloxy, heptyloxy and nonyloxy.

Preferable examples of the cycloalkyloxy group include C₃₋₁₀ cycloalkyloxy group such as cyclobutoxy, cyclopentyloxy and cyclohexyloxy.

Preferable examples of the alkenyloxy group include C₂₋₁₀ alkenyloxy group such as allyloxy, crotyloxy, 2-pentenyloxy and 3-hexenyloxy.

Preferable examples of the cycloalkenyloxy group include C₅₋₁₀ cycloalkenyloxy group such as 2-cyclopentenyloxy and 2-cyclohexenyloxy.

Preferable examples of the aralkyloxy group include C₇₋₂₀ aralkyloxy group such as C₆₋₁₄ aryl-C₁₋₆ alkoxy group such as phenyl-C₁₋₆ alkoxy group (e.g. benzyloxy and phenethyloxy), naphthyl-C₁₋₆ alkoxy group.

Preferable examples of the aryloxy group include C₆₋₁₄ aryloxy group optionally substituted with C₁₋₃ alkyl group, C₁₋₃ alkoxy group, halogen, nitro group, hydroxyl group or amino group, which are exemplified, by phenoxy and 4-chlorophenoxy. Preferable examples of the acyloxy group include C₂₋₁₅ acyloxy group such as C₂₋₇ alkanoyloxy group (e.g. acetyloxy, propionyloxy, butyryloxy and isobutyryloxy), C₆₋₁₄ aryl-carbonyloxy (e.g. benzoyloxy and naphthoyloxy).

Examples of the optionally substituted thiol group include mercapto group, alkylthio group, cycloalkylthio group, alkenylthio group, aralkylthio group, arylthio group, heteroarylthio group, heteroarylalkylthio group and acylthio group.

Preferable examples of the alkylthio group include C₁₋₁₀ alkylthio group such as methylthio, ethylthio, propylthio, isopropylthio, butylthio, isobutylthio, sec-butylthio, tert-butylthio, pentylthio, isopentylthio, neopentylthio, hexylthio, heptylthio and nonylthio.

Preferable examples of the cycloalkylthio group include C₃₋₁₀ cycloalkylthio group such as cyclobutylthio, cyclopentylthio and cyclohexylthio.

Preferable examples of the alkenylthio group include C₂₋₁₀ alkenylthio group such as allylthio, crotylthio, 2-pentenylthio and 3-hexenylthio.

Preferable examples of the aralkylthio group include C₇₋₂₀ aralkylthio group such as C₆₋₁₄ arylthio group, exemplified, more specifically, by phenyl-C₁₋₆ alkylthio (e.g. benzylthio and phenethylthio), and naphthyl-C₁₋₆ alkylthio.

Preferable examples of the arylthio group include C₆₋₁₄ arylthio group optionally substituted with C₁₋₃ alkyl group, C₁₋₃ alkoxy group, halogen, nitro group, hydroxyl group or amino group, such as phenylthio, naphthylthio and 4-chlorophenylthio.

As the heteroarylthio group, mention is made of, for example, the mercapto group substituted with aromatic heterocyclic group as mentioned above, especially preferable one being pyridylthio (e.g. 2-pyridylthio, 3-pyridylthio), imidazolylthio (e.g. 2-imidazolylthio) or triazoylthio (e.g. 1,2,4-triazol-5-ylthio).

As the heteroarylalkylthio group, mention is made of, or example, the above-mentioned alkylthio group substituted with the above-mentioned aromatic heterocyclic group.

Preferable examples of the heteroarylthio group include pyridyl-C₁₋₆ alkylthio group (e.g. 2-pyridylmethylthio and 3-pyridylmethylthio).

Preferable examples of the acylthio group include C₂₋₁₅ acylthio group, such as C₂₋₇ alkanoylthio group (e.g. acetylthio, propionylthio, butyrylthio and isobutyrylthio), C₆₋₁₄ aryl-carbonylthio (e.g. benzoylthio and naphthoylthio).

As optionally esterified or amidated carboxyl group, mention is made of carboxyl group, esterified carboxyl group and amidated carboxyl group.

Examples of the esterified carboxyl group include alkoxycarbonyl group, aralkyloxycarbonyl group, aryloxycarbonyl group and heteroarylalkyloxycarbonyl group.

Preferable examples of the alkoxycarbonyl group include C₂₋₇ alkoxycarbonyl group such as methoxycarbonyl, ethoxycarbonyl, propoxycarbonyl and butoxycarbonyl.

Preferable examples of the aralkyloxycarbonyl group include C₈₋₂₁ aralkyloxycarbonyl group such as phenyl-C₂₋₇ alkoxycarbonyl (e.g. benzyloxycarbonyl) and naphthyl-C₂₋₇ alkoxycarbonyl.

Preferable examples of the aryloxycarbonyl group include C₇₋₁₅ aryloxycarbonyl group optionally substituted with C₁₋₃ alkyl group, C₁₋₃ alkoxy group, halogen, nitro group, hydroxyl group and amino group, such as phenoxycarbonyl and p-tolyloxycarbonyl.

As the heteroarylalkyloxycarbonyl, mention is made of, for example, the above-mentioned alkoxycarbonyl group substituted with the above-mentioned aromatic heterocyclic group. Preferable examples of the heteroarylalkyloxycarbonyl group include pyridyl-C₂₋₇ alkoxycarbonyl group (e.g. 2-pyridylmethoxycarbonyl and 3-pyridylmethoxycarbonyl).

As the amidated carboxyl group, mention is made of the group represented by the formula: —CON(R¹)(R²) [wherein R¹ and R² are the same or different and each stands for hydrogen atom, optionally substituted hydrocarbon group or optionally substituted heterocyclic group]. As the hydrocarbon group in optionally substituted hydrocarbon group shown by R¹ or R², mention is made of the aliphatic hydrocarbon group, alicyclic hydrocarbon group and aromatic hydrocarbon group exemplified as the substituents on the aromatic heterocyclic group shown by R. As the heterocyclic group in the optionally substituted heterocyclic group shown by R¹ or R², mention is made of the aromatic heterocyclic group exemplified as the substituents on the aromatic heterocyclic group shown by R. As substituents on the hydrocarbon group or heterocyclic group in R¹ or R², mention is made of 1 to 3 substituents selected from halogen atoms (e.g. chlorine, fluorine, bromine and iodine), C₁₋₆ alkyl groups and C₁₋₆ alkoxy groups.

In the general formula (I), when the substituent on aromatic heterocyclic group represented by R, the aromatic azole group represented by the formula

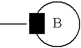

or ring A is alicyclic hydrocarbon group, aromatic hydrocarbon group, aliphatic hydrocarbon group substituted with aromatic hydrocarbon group, aromatic heterocyclic group, non-aromatic heterocyclic group or aliphatic hydrocarbon group substituted with aromatic heterocyclic group, the said alicyclic hydrocarbon group, aromatic hydrocarbon group, the aromatic hydrocarbon group in the aliphatic hydrocarbon group substituted with aromatic hydrocarbon group, aromatic heterocyclic group, non-aromatic hydrocarbon group or the aromatic heterocyclic group in the aliphatic hydrocarbon group substituted with aromatic heterocyclic group may optionally have further 1 to 3 (preferably 1 or 2) substituents on respectively substitutable positions. Examples of such substituents include optionally substituted C₁₋₆ alkyl group, C₂₋₆ alkenyl group, C₂₋₆ alkynyl group, C₃₋₁₀ cycloalkyl group, C₅₋₁₀ cycloalkenyl group, C₆₋₁₄ aryl group (e.g. phenyl and naphthyl), aromatic heterocyclic group (e.g. thienyl, furyl, pyridyl, oxazolyl, thiazolyl and tetrazolyl), non-aromatic heterocyclic group (e.g. tetrahydrofuryl, morpholinyl, piperidyl, pyrrolidyl and piperazinyl), C₇₋₂₀ aralkyl group (e.g. phenyl-C₁₋₆ alkyl group, naphthyl-C₁₋₆ alkyl group), amino group, N-mono(C₁₋₆)alkylamino group, N,N-di(C₁₋₆)alkylamino group, C₂₋₇ acylamino group (e.g. C₂₋₇ alkanoylamino group such as acetylamino and propionylamino, and benzoylamino group), amidino group, C₂₋₇ acyl group (e.g. C₂₋₇ alkanoyl group and benzoyl group), carbamoyl group, N-mono(C₁₋₆) alkylcarbamoyl group, N,N-di(C₁₋₆)alkylcarbamoyl group, sulfamoyl group, N-mono(C₁₋₆)alkylsulfamoyl group, N,N-di(C₁₋₆)alkylsulfamoyl group, carboxyl group, C₂₋₇ alkoxycarbonyl group, C₈₋₂₁ aralkyloxycarbonyl group (e.g. phenyl-C₂₋₇ alkoxycarbonyl and naphthyl-C₂₋₇ alkoxycarbonyl), hydroxyl group, optionally substituted C₁₋₆ alkoxy group, C₂₋₆ alkenyloxy group, C₃₋₁₀ cycloalkyloxy group, C₅₋₁₀ cycloalkenyloxy group, C₇₋₂₀ aralkyloxy group (e.g. phenyl-C₁₋₆ alkoxy group, naphthyl-C₁₋₆ alkoxy group), C₆₋₁₄ aryloxy group (e.g. phenoxy and naphthyloxy), mercapto group, C₁₋₆ alkylthio group, C₃₋₁₀ cycloalkylthio group, C₇₋₂₀ aralkylthio group (e.g. phenyl-C₁₋₆ alkyl group and naphthyl-C₁₋₆ alkylthio group), C₆₋₁₄ arylthio group (e.g. phenylthio and naphthylthio group), sulfo group, cyano group, azide group, nitro group, nitroso group, and halogen atoms (e.g. fluorine, chlorine, bromine and iodine).

As substituents in the above-mentioned optionally substituted C₁₋₆ alkoxy group and optionally substituted C₁₋₆ alkyl group, mention is made of, for example, 1 to 3 substituents selected from halogen atoms (e.g. fluorine, chlorine, bromine and iodine), hydroxyl group and C₁₋₆ alkoxy groups.

As the substituted C₁₋₆ alkoxy group, mention is made of, for example, trifluoromethoxy, difluoromethoxy, 2,2,2-trifluoroethoxy and 1,1-difluoroethoxy.

As the substituted C₁₋₆ alkyl group, mention is made of, for example, trifluoromethyl, difluoromethyl, 2,2,2-trifluoroethyl, trichloromethyl, hydroxymethyl, methoxymethyl, ethoxymethyl, 2-methoxyethyl and 2,2-dimethoxyethyl.

In the general formula (I), when the substituent on the aromatic heterocyclic group represented by R, the aromatic azole group represented by the formula

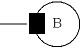

or ring A is aliphatic hydrocarbon group, aliphatic hydrocarbon group substituted with aromatic hydrocarbon group, or aliphatic hydrocarbon group substituted with aromatic heterocyclic group, said aliphatic hydrocarbon group, the aliphatic hydrocarbon group in the aliphatic hydrocarbon group substituted with aromatic hydrocarbon group, or the aliphatic hydrocarbon group in the aliphatic hydrocarbon group substituted with aromatic heterocyclic group may have further 1 to 3 (preferably 1 or 2) substituents at respectively substitutable positions. Examples of these substituents include non-aromatic heterocyclic group (e.g. tetrahydrofuryl, morpholinyl, piperidyl, pyrrolidyl and piperazinyl), amino group, N-mono(C₁₋₆)alkylamino group, N,N-di(C₁₋₆)alkylamino group, C₂₋₇ acylamino group (e.g. C₂₋₈ alkanoylamino group such as acetylamino and propionylamino, and benzoylamino group), amidino group, C₂₋₇ acyl group (e.g. C₂₋₇ alkanoyl group and benzoyl group), carbamoyl group, N-mono(C₁₋₆)alkylcarbamoyl group, N,N-di(C₁₋₆)alkylcarbamoyl group, sulfamoyl group, N-mono(C₁₋₆)alkylsulfamoyl group, N,N-di(C₁₋₆)alkylsulfamoyl group, carboxyl group, C₂₋₇ alkoxycarbonyl group, C₈₋₂₁ aralkyloxycarbonyl group (e.g. phenyl-C₂₋₇ alkoxycarbonyl group and naphthyl-C₂₋₇ alkoxycarbonyl group), hydroxyl group, optionally substituted C₁₋₆ alkoxy group, C₂₋₆ alkenyloxy group, C₃₋₁₀ cycloalkyloxy group, C₅₋₁₀ cycloalkenyloxy group, C₇₋₂₀ aralkyloxy group (e.g. phenyl-C₁₋₆ alkoxy group and naphthyl-C₁₋₆ alkoxy group), C₆₋₁₄ aryloxy group (e.g. phenoxy and naphthyloxy), mercapto group, C₁₋₆ alkylthio group, C₃₋₁₀ cycloalkylthio group, C₇₋₂₀ aralkylthio group (e.g. phenyl-C₁₋₆ alkyl group, naphthyl-C₁₋₆ alkylthio group), C₆₋₁₄ arylthio group (e.g. phenylthio and naphthylthio), sulfo group, cyano group, azide group, nitro group, nitroso group, halogen atoms (e.g. fluorine, chlorine, bromine and iodine).

As the substituents in the above-mentioned optionally substituted C₁₋₆ alkoxy group, mention is made of, for example, 1 to 3 substituents selected from halogen atoms (e.g. fluorine, chlorine, bromine and iodine), hydroxyl group, and C1.6 alkoxy groups.

As the above-mentioned substituted C₁₋₆ alkoxy group, mention is made of, for example, trifluoromethoxy, difluoromethoxy, 2,2,2-trifluoroethoxy and 1,1-difluoroethoxy.

Preferable examples of R are oxazolyl group, benzoxazolyl group or thiazolyl group optionally respectively substituted with 1 or 2 substituents selected from (i) aryl group (e.g. phenyl group and naphthyl group) optionally substituted with 1 or 2 substituents selected from hydroxyl group, alkoxy group (e.g. C₁₋₆ alkoxy group), arylalkoxy group (e.g. phenyl-C₁₋₆ alkoxy group), alkyl group (e.g. C₁₋₆ alkyl group), cyano group, halogen atom and tetrazolyl group, (ii) alkyl group (e.g. C₁₋₁₀ alkyl group), (iii) hydroxyalkyl group (e.g. hydroxy-C₁₋₁₀ alkyl group), (iv) alkoxycarbonylalkyl group (e.g. C₂₋₇ alkoxycarbonyl-C₁₋₁₀ alkyl group), (v) alkyl group substituted with 1 or 2 aryl groups (e.g. C₁₋₆ alkyl group substituted with 1 or 2 phenyl groups), (vi) alkenyl group substituted with 1 or 2 aryl groups (e.g. C₂₋₆ alkenyl group substituted with 1 or 2 phenyl groups), (vii) cycloalkyl group (e.g. C₃₋₁₀ cycloalkyl group), (viii) partially saturated naphthyl group (e.g. dihydronaphthyl group), (ix) thienyl or furyl group optionally substituted with 1 or 2 substituents selected from hydroxyl group, alkoxy group, arylalkoxy group, alkyl group, cyano group, allyl group and halogen atom, (x) benzofuranyl group and (xi) benzothienyl, and oxazolyl group substituted with arylalkenyl group (e.g. phenyl-C₂₋₆ alkenyl group) and oxazolyl group substituted with arylalkoxy-aryl group (e.g. phenyl-C₁₋₆ alkoxy-phenyl group) are more preferable.

Preferable examples of the aromatic azole group represented by the formula

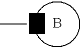

include pyrrolyl group, imidazolyl group, pyrazolyl group, triazolyl group, tetrazolyl group or benzimidazolyl group respectively substituted with 1 or 2 substituents selected from (i) alkyl group (e.g. C₁₋₁₀ alkyl group), (ii) aryl group (e.g. phenyl group), (iii) hydroxyalkyl group (e.g. hydroxy-C₁₋₁₀ alkyl group), (iv) carboxyl group, (v) alkoxycarbonyl group (e.g. C₂₋₇ alkoxycarbonyl group) and (vi) carbamoyl group, and imidazolyl group and triazolyl group are more preferable.

The ring A forms, depending on the kind of Y (CH or N), optionally substituted benzene ring or optionally substituted pyridine ring. As preferable examples, mention is made of optionally substituted benzene ring. More preferable examples include benzene ring or pyridine ring, which are optionally substituted with 1 or 2 C₁₋₆ alkoxy groups.

Preferable examples of the group represented by the formula:

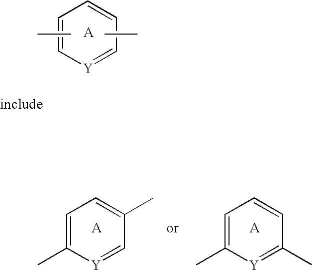

and most preferable ones are 1,3-phenylene group or 1,4-phenylene group.

X stands for an oxygen atom (O), an optionally oxidized sulfur atom [S(O)ₖ (k means an integer of 0 to 2)], —C(═O)— or —CH(OH)—, and the preferable examples include oxygen atom.

The symbol p denotes an integer of 0 to 10, preferable 0 to 6, more preferably 3 to 5.

The symbol q means an integer of 1 to 5, preferably I.

As a specific example of compound (I), the compounds produced in Example of JP-A-11-60571 are mentioned. Particularly, (i) 1-[4-[4-[2-[(E)-2-phenylethenyl]-4-oxazolylmethoxy]phenyl]butyl-1,2,4-triazole, (ii) 4-[4-[4-(1-imidazolyl)butyl]phenoxymethyl]-2-[(E)-2-phenylethenyl]oxazole (iii) 4-[4-[3-(1-imidazolyl)propyl]phenoxymethyl]-2-[(E)-2-phenylethenyl]oxazole, (iv) 4-[3-[3-(1-imidazolyl)propyl]phenoxymethyl]-2-[(E)-2-phenylethenyl]oxazole, (v) 2-(4-benzyloxyphenyl)-4-[4-[4-[3-(1-imidazolyl)propyl]phenoxymethyl]oxazole, (vi) 4-[4-[3-(1-imidazolyl)propyl]phenoxymethyl]-2-(2-thienyl)oxazole, (vii) 4-[4-[3-(1-imidazolyl)propyl]phenoxymethyl]-2-(5-methyl-2-thienyl)oxazole, (viii) 2-(5-chloro-2-thienyl)-4-[4-[3-(1-imidazolyl)propyl]phenoxymethyl]oxazole, (ix) 4-[4-[3-(1-imidazolyl)propyl]phenoxymethyl]-2-(2-thienyl)thiazole, (x) 5-[4-[3-(1-imidazolyl)propyl]phenoxymethyl]-2-(2-thienyl)benzoxazole and the like are preferred.

As the above-mentioned compound (I), for example, compound (II) represented by the formula

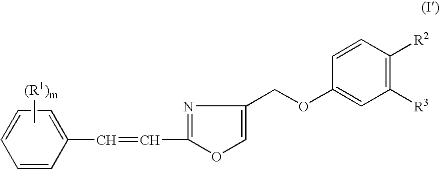

wherein m is 1 or 2;

R¹ is a halogen atom or an optionally halogenated C₁₋₂ alkyl group;

one of R² and R³ is a hydrogen atom and the other is a group represented by the formula:

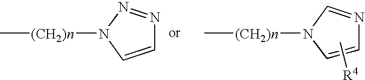

wherein n is 3 or 4; R⁴ is a C₁₋₄ alkyl group substituted by 1 or 2 hydroxy groups, and the like is preferable.

With respect to the formula (I′) above, the “halogen atom” represented by R¹ is exemplified by fluoro, chloro, bromo, and iodo. In particular, fluoro is preferred.

The “halogen” of the “optionally halogenated C₁₋₂ alkyl group” represented by R¹ is exemplified by fluoro, chloro, bromo, and iodo. In particular, fluoro is preferred.

The “C₁₋₂ alkyl group” of the “optionally halogenated C₁₋₂ alkyl group”represented by R¹ is exemplified by methyl and ethyl, and methyl is preferred.

Said “C₁₋₂ alkyl group” may have 1 to 3, preferably 2 or 3, halogens mentioned above at any substitutable positions; hen 2 or more such halogens are present, they may be identical or different.

As specific examples of said “optionally halogenated C₁₋₂ alkyl group”, there may be mentioned methyl, ethyl, and trifluoromethyl.

R¹ is preferably a halogen atom or a halogenated C₁₋₂ alkyl group, and fluoro and trifluoromethyl are more preferable.

When m is 2, the R¹ groups may be different.

The group represented by R² or R³ for the formula:

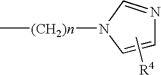

wherein R⁴ has the same meanings as defined above, is preferably a group represented by the formula:

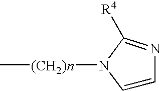

wherein R⁴ has the same meaning as defined above.

As examples of the “C₁₋₄ alkyl group” of the “C₁₋₄ alkyl group substituted by 1 or 2 hydroxy groups” represented by R⁴, there may be mentioned methyl, ethyl, propyl, isopropyl, butyl, isobutyl, sec-butyl, and tert-butyl. In particular, ethyl, propyl, etc. are preferred.

As examples of said “C₁₋₄ alkyl group substituted by 1 or 2 hydroxy groups”, there may be mentioned 2-hydroxyethyl, 2,3-dihydroxypropyl, and 1,3-dihydroxypropyl. In particular, 2,3-dihydroxypropyl is preferred.

With respect to the formula above, it is preferable that R² is a group represented by the formula:

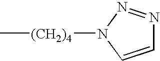

and R³ is a hydrogen atom.

It is also preferable that R² is a hydrogen atom and R³ is a group represented by the formula:

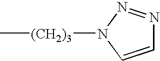

It is also preferable that R² is a group represented by the formula:

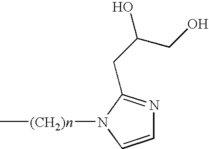

wherein n has the same meaning as defined above, and R³ is a hydrogen atom, with n being more preferably 4.

As a preferable example of Compound (I′), there may be mentioned a compound represented by the formula:

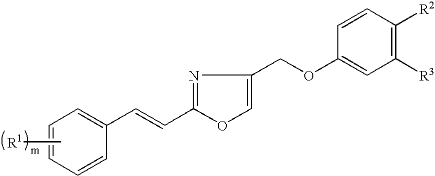

wherein the symbols have the same meaning as defined above, or a salt thereof.

Of Compound (I), a compound wherein m is 1; R¹ is 4-trifluoromethyl; R² is a group represented by the formula:

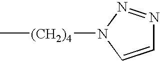

and R³ is a hydrogen atom, or a salt thereof is preferred.

As specific examples of Compound (I′), there may be mentioned

(i) 1-(4-{4-[(2-{(E)-2-[4-(trifluoromethyl)phenyl]ethenyl}-1,3-oxazol-4-yl)methoxy]phenyl}butyl)-1H-1,2,3-triazole,

(ii) 1-(3-{3-[(2-{(E)-2-[4-(trifluoromethyl)phenyl]ethenyl}-1,3-oxazol-4-yl)methoxy]phenyl}propyl)-1H-1,2,3-triazole,

(iii) 3-(1-{4-[4-({2-[(E)-2-(2,4-difluorophenyl)ethenyl]-1,3-oxazol-4-yl}methoxy)phenyl]butyl}-1H-imidazol-2-yl)-1,2-propanediol, etc.

As the salt of Compound (I) of the present invention, pharmaceutically acceptable salts are preferred, including salts with inorganic bases, salts with organic bases, salts with inorganic acids, salts with organic acids, and salts with basic or acidic amino acids. As preferable examples of salts with inorganic bases, there may be mentioned alkali metal salts such as sodium salt and potassium salt; alkaline earth metal salts such as calcium salt and magnesium salt; aluminum salt; and ammonium salt. As preferable examples of salts with organic bases, there may be mentioned salts with trimethylamine, triethylamine, pyridine, picoline, ethanolamine, diethanolamine, triethanolamine, dicyclohexylamine, N,N′-dibenzylethylenediamine, etc. As preferable examples of salts with inorganic acids, there may be mentioned salts with hydrochloric acid, hydrobromic acid, nitric acid, sulfuric acid, phosphoric acid, etc. As preferable examples of salts with organic acids, there may be mentioned salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc. As preferable examples of salts with basic amino acids, there may be mentioned salts with arginine, lysine, ornithine, etc.; as preferable examples of salts with acidic amino acids, there may be mentioned salts with aspartic acid, glutamic acid, etc.

In Compound (I), two kinds, i.e., (Z)-ethenyl configuration and (E)-ethenyl configuration, are present; these isomers are included in the scope of the present invention, whether they are present in the form of simple substances or mixtures.

Furthermore, when Compound (I) has asymmetric carbons, optical isomers exist; these isomers are included in the scope of the present invention, whether they are present in the form of simple substance or mixtures.

Compound (I) or a salt thereof is obtained by commonly known methods, e.g., a method based on the method described in Japanese Patent Unexamined Publication No. 60571/1999.

Specifically, compound (I′) or a salt thereof is obtained by, for example, the methods schematized by Reaction Formulas A through H below.

The symbols for the compounds given in the schemes for the reaction formulas below have the same definitions as those shown above. The compounds shown in the reaction formulas include salts thereof.

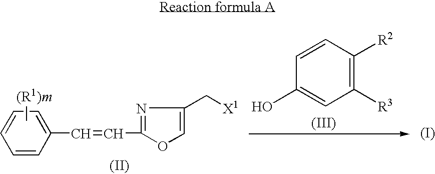

As examples of the “leaving group” represented by X¹, there may be mentioned halogen (e.g., chloro, bromo) or group represented by the formula:

—OSO₂R⁵ wherein R⁵ is an alkyl or an aryl optionally having substituent.

As examples of the “alkyl” represented by R⁵, there may e mentioned C₁₋₆ alkyl such as methyl, ethyl, and propyl.

As examples of the “aryl” of the “aryl optionally having substituent” represented by R⁵, there may be mentioned C₆₋₁₄ aryls such as phenyl.

The “substituent” of the “aryl optionally having substituent” represented by R⁵ is exemplified by C₁₋₆ alkyls such as methyl, ethyl, and propyl.

As specific examples of said “aryl optionally having substituent,” there may be mentioned phenyl optionally having C₁₋₆ alkyl (e.g., p-tolyl).

Compound (II) and Compound (III) are reacted to yield Compound (I′).

This condensation reaction is usually carried out in the presence of a base between Compound (II) and Compound (III).

As examples of said “base”, there may be mentioned alkali metal or alkaline earth metal hydroxides (e.g., sodium hydroxide, potassium hydroxide), alkali metal or alkaline earth metal carbonates (e.g., sodium hydrogen carbonate, sodium carbonate, potassium carbonate), amines (e.g., pyridine, triethylamine, N,N-dimethylaniline), alkali metal or alkaline earth metal hydrides (e.g., sodium hydride, potassium hydride, calcium hydride), and alkali metal or alkaline earth metal lower alkoxides (e.g., sodium methoxide, sodium ethoxide, potassium tert-butoxide).

The amount of “base” used is preferably about 1 to 5 mol per mol of Compound (II).

The amount of “Compound (III)” used is preferably about 0.5 to 5 mol per mol of Compound (II).

This reaction is advantageously carried out in the presence of a solvent which does not interfere with the reaction. Said solvent is not subject to limitation, as long as the reaction proceeds; as examples of this solvent, aromatic hydrocarbons, ethers, ketones, halogenated hydrocarbons, amides, sulfoxides or mixtures of two or more kinds thereof may be used.

Reaction temperature is normally 50 to +150° C., preferably about −10 to +100° C. Reaction time is normally 0.5 to 48 hours.

Compound (II) can be produced by a commonly known method or a modification thereof, e.g., Compound (IIa), wherein X is chloro, can be produced by the method shown by Reaction Formula B below, or the like.

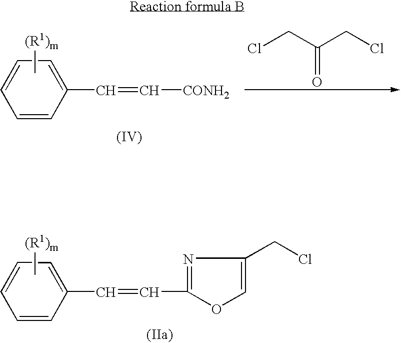

Compound-(IV) and 1,3-dichloroacetone are subjected to a condensation/dehydration reaction to yield Compound (IIa).

If commercially available, Compound (IV) may be used as a commercial product as is, or may be produced by a commonly known method, a modification thereof, or the like.

The amount of “1,3-dichloroacetone” used is about 1 equivalent to a large excess (amount of solvent) relative to Compound (IV).

This reaction is advantageously carried out in the absence of solvent or in the presence of solvent which does not interfere with the reaction. Said solvent is not subject to limitation, as long as the reaction proceeds; as examples of this solvent, aromatic hydrocarbons, ethers, ketones, halogenated hydrocarbons or mixtures of two or more kinds thereof may be used.

Reaction temperature is normally 50 to 150° C., preferably about 60 to 120° C. Reaction time is normally 0.5 to 48 hours.

Although the product can be used for the next reaction in the form of a reaction mixture as-is, or in the form of a crude product, it can also be isolated from the reaction mixture by a conventional method.

Of Compound (III), Compound (IIIa), wherein R³ is a hydrogen atom, can be produced by a commonly known method or a modification thereof, e.g., the method shown by Reaction Formula C below.

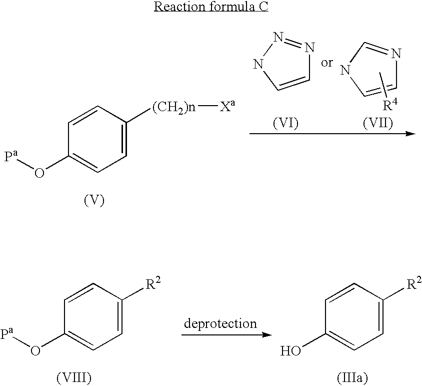

With respect to the formula above, p^{a} is a hydrogen atom or a protective group; X^{a} is a leaving group.

As examples of the “protective group” represented by p^{a}, there may be mentioned alkyls (e.g., C₁₋₆ alkyls such as methyl and ethyl), phenyl-C₁₋₆ alkyls (e.g., benzyl), C₁₋₆ alkyl-carbonyl, alkyl-substituted silyl (e.g., trimethylsilyl, tert-butyldimethylsilyl).

As examples of the “leaving group” represented by X^{a}, there may be mentioned the same examples as those of the “leaving group” represented by X¹ above.

By condensing Compound (V) and Compound (VI) or Compound (VII) to yield Compound (VIII), which is subjected to a deprotecting reaction as necessary, Compound (IIIa) is obtained.

If commercially available, each of Compound (V), Compound (VI) and Compound (VII) may be used as a commercial product as is, or may be produced by a commonly known method, a modification thereof, or the like.

Said “condensation reaction” is normally carried out in the presence of a base in a solvent which does not interfere with the reaction.

Said “base” is exemplified by the bases described in detail with respect to Reaction Formula above.

The amount of “base” used is preferably about 1 to 5 mol per mol of Compound (V).

The amount of “Compound (VI) or Compound (VII)” used is preferably about 0.5 to 5 mol per mol of Compound (V).

Said solvent is not subject to limitation, as long as the reaction proceeds; as examples of this solvent, aromatic hydrocarbons, ethers, ketones, halogenated hydrocarbons, amides, sulfoxides or mixtures of two or more kinds thereof may be used.

The reaction temperature is normally −50 to +150° C., preferably about −10 to +100° C. Reaction time is about 0.5 to 48 hours.

Although Compound (VIII) obtained can be used for the next reaction in the form of a reaction mixture as-is, or in the form of a crude product, it can also be isolated from the reaction mixture by a conventional method.

Said “deprotection reaction” can be carried out by an appropriately selected conventional method.

When p^{a} is an alkyl, for example, Compound (VIII) is subjected to a treatment with an acid (e.g., mineral acid such as hydrobromic acid, or Lewis acid such as titanium tetrachloride).

When p^{a} is a phenyl-C₁₋₆ alkyl, for example, Compound (VIII) is subjected to a hydrogenation reaction.

When P^{a} is an alkyl-substituted silyl, for example, Compound (VIII) is reacted with a fluoride (e.g., tetrabutylammonium fluoride).

Although Compound. (IIIa) obtained can be used for the next reaction in the form of a reaction mixture as-is, or in the form of a crude product, it can also be isolated from the reaction mixture by a conventional method.

Of Compound (III), Compound (IIIb), wherein R² is a hydrogen atom, can be produced by a commonly known method or a modification thereof, e.g., the method shown by Reaction Formula D below.

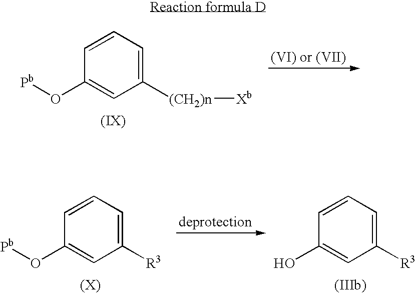

With respect to the formula above, p^{b} is a hydrogen atom or a protective group; X is a leaving group.

The “protective group” represented by p^{b} is the same as the “protective-group” represented by p^{a} above.

The “leaving group” represented by X^{b} is, for example, the same as the leaving group represented by X¹ above.

In the same manner as the method described with respect to Reaction Formula C above, Compound (IX) and Compound (VI) or Compound (VII) are condensed to yield Compound (X), which is then subjected to a deprotection reaction as necessary to yield Compound (IIIb).

If commercially available, Compound (IX) may be used as a commercial product as is, or may be produced by a commonly known method, a modification thereof, or the like.

Of Compound (I′), Compound (Ia′), wherein R³ is a hydrogen atom, can also be produced by the method shown by Reaction Formula E below.

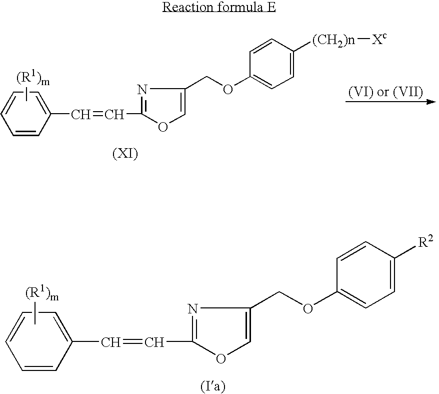

With respect to the formula above, X^{c} is a leaving group.

The “leaving group” represented by X^{c} is, for example, the same as the leaving group represented by X¹ above.

Compound (XI) and-Compound (VI) or Compound (VII) are reacted to yield Compound (Ia).

This condensation reaction is normally carried out in the presence of a base between Compound (XI) and Compound (VI) or Compound (VII).

Said “base” is exemplified by the base described in detail with respect to Reaction Formula A above.

The amount of “base” used is preferably about 1 to 5 mol per mol of Compound (XI).

The amount of each of “Compound (VI)” and “Compound (VII)” used is preferably about 0.5 to 5 mol per mol of Compound (XI).

This reaction is advantageously carried out in the presence of solvent that does not interfere with the reaction. Said solvent is not subject to limitation, as long as the reaction proceeds, and is exemplified by aromatic hydrocarbons, ethers, ketones, halogenated hydrocarbons, amides, sulfoxides, or mixtures of two or more kinds thereof.

The reaction temperature is normally −20 to +150° C., preferably about −10 to +100° C. The reaction time is normally 0.5 to 48 hours.

Compound (XI) can be produced by a commonly known method or a modification thereof, e.g., the method shown by Reaction Formula F below.

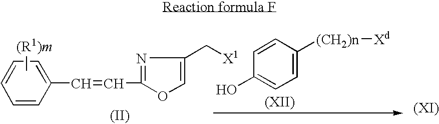

With respect to the formula above, X^{d} is a leaving group.

The “leaving group” represented by X^{d} is, for example, the same as the leaving group represented by X¹ above, and is preferably a leaving group which is less reactive than X¹.

In the same manner as the method described with respect to Reaction Formula A above, Compound (II) and Compound (XII) are reacted to yield Compound (XI).

If commercially available, Compound (XII) may be used as a commercial product as is, or may be produced by a commonly known method, a modification thereof, or the like.

Of Compound (I′), Compound (Ib′), wherein R² is a hydrogen atom, can also be produced by the method shown by Reaction Formula G below.

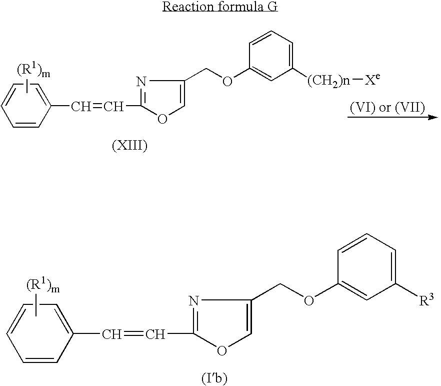

With respect to the formula above, X^{e} is a leaving group.

The “leaving group” represented by X^{e} is, for example, the same as the leaving group represented by X¹ above.

In the same manner as the method described with respect to Reaction Formula E above, Compound (XIII) and Compound (VI) or Compound (VII) are reacted to yield Compound (Ib′).

Compound (XIII) can be produced by a commonly known method or a modification thereof, e.g., the method shown by Reaction Formula H below.

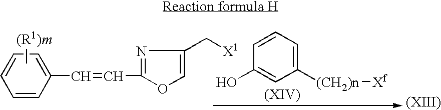

With respect to the formula above, X^{f} is a leaving group.

The “leaving group” represented by X^{f} is, for example, the same as the leaving group represented by X¹ above, and is preferably a leaving group which is less reactive than X¹.

In the same manner as the method described with respect to Reaction Formula A above, Compound (II) and Compound (XIV) are reacted to yield Compound (XIII).

If commercially available, Compound (XIV) may be used as a commercial product as is, or may be produced by a commonly known method, a modification thereof, or the like.

As the aforementioned “aromatic hydrocarbons”, for example, benzene, toluene, xylene, etc. are used.

As the aforementioned “ethers” for example, tetrahydrofuran, dioxane, etc. are used.

As the aforementioned “ketones”, for example, acetone, 2-butanone, etc. are used.

As the aforementioned “halogenated hydrocarbons”, for example, chloroform, dichloromethane, etc. are used.

As the aforementioned “amides”, for example, N,N-dimethylformamide etc. are used.

As the aforementioned “sulfoxides”, for example, dimethylsulfoxide etc. are used.

In each reaction mentioned above, if the product is obtained as a free form, it can be converted into a salt thereof by a conventional method; if the product is obtained as a salt, it can be converted into a free form thereof by a conventional method.

In the reactions mentioned above, if amino (NH₂), hydroxy (OH), carboxyl (COOH), or the like is contained in a substituent, the starting material may have these groups protected and the protective groups may be removed by a commonly known method after the reaction to produce the desired product. As amino-protecting groups, there may be mentioned acyls (e.g., C₁₋₆ alkyl-carbonyls such as acetyl; benzyloxycarbonyl; C₁₋₆ alkoxy-carbonyls such as tert-butoxycarbonyl; phthaloyl; formyl). As examples of hydroxy-protecting groups, there may be mentioned C₁₋₆ alkyls (e.g., methyl, ethyl), phenyl-C₁₋₆ alkyls (e.g., benzyl), C₁₋₆ alkylcarbonyls (e.g., acetyl), benzoyl, and alkyl-substituted silyls (e.g., trimethylsilyl, tert-butyldimethylsilyl). As examples of carboxyl-protecting groups, there may be mentioned C₁₋₆ alkyls (e.g., methyl, ethyl), and phenyl-C₁₋₆ alkyls (e.g., benzyl).

Compound (I′) [Including (I′a) and (I′b)] thus obtained can be isolated and purified by commonly known means for separation, e.g., concentration, concentration under reduced pressure, solvent extraction, crystallization, recrystallization, re-dissolution, and chromatography.

If Compound (I′) is obtained as a free form, it can be converted into a desired salt by a commonly known method or a modification thereof; conversely, if Compound (I′) is obtained as a salt, it can be converted into a free form or another desired salt by a commonly known method or a modification thereof.

Compound (I) may be a hydrate or a non-hydrate.

When Compound (I) is obtained as a mixture of optical isomers, the desired (R)-configuration or (S)-configuration can be separated by a commonly known means of optical resolution.

Compound (I) may be labeled with an isotope (e.g., ³H, ¹⁴C) or the like.

A pro-drug of the compound (I) or a salt thereof (hereinafter referred to as the compound (I)) means a compound which is converted to the compound (I) under physiological conditions or by reaction with an enzyme, gastric acid, etc. in the living body, that is, a compound which is converted to the compound (I) with oxidation, reduction, hydrolysis, etc. y an enzyme; a compound which is converted to the compound (I) with hydrolysis, etc. by gastric acid. Examples of the pro-drug of the compound (I) include a compound wherein a hydroxy group of the compound (I) is substituted with acyl;

alkyl, phosphoric acid, boric acid, etc. (e.g. a compound wherein a hydroxy group of the compound (I) is substituted with acetyl, palmitoyl, propanoyl, pivaloyl, succinyl, fumaryl, alanyl, dimethylaminomethylcarbonyl, etc.) etc. These pro-drugs can be produced by per se known methods from the compound (I).

The pro-drug of the compound (I) may be a compound which is converted into the compound (I) under the physiological conditions as described in “Pharmaceutical Research and Development”, Vol. 7 (Drug Design), pages 163-198 published in 1990 by Hirokawa Publishing Co. (Tokyo, Japan).

The compound (I)(including prodrug) is of low toxicity and can be used as a pharmaceutical as-is, or used as pharmaceutical composition in a mixture with a commonly known pharmaceutically acceptable carrier etc. in mammals (e.g., humans, horses, bovines, dogs, cats, rats, mice, rabbits, pigs, monkeys).

In addition to the compound (I), said pharmaceutical composition may contain other active ingredients, e.g., the following hormone therapy agents, anti-cancer agents (e.g., chemotherapy agents, immunotherapy agents, or drugs which inhibit the activity of cell growth factors and receptors thereof) and the like.

As a pharmaceutical for mammals such as humans, the compound (I) can be administered orally in the form of, for example, tablets, capsules (including soft capsules and microcapsules), powders, and granules, or non-orally in the form of injections, suppositories, and pellets. Examples of the parenteral administration route include intravenous, intramuscular, subcutaneous, intra-organ, intranasal, intradermal, instillation, intracerebral, intrarectal, intravaginal, intraperitoneal, intratumoral, juxtaposition of tumor and administration directly to the lesion.

The dose of the compound (I) varies depending on the route of administration, symptoms, etc. For example, when it is administered orally as an anticancer agent to a patient (body weight 40 to 80 kg) with breast cancer or prostate cancer, its dose is, for example, 0.5 to 100 mg/kg body weight per day, preferably 1 to 50 mg/kg body weight per day, and more preferably 1 to 25 mg/kg body weight per day. This amount may be administered once or in 2 to 3 divided portions daily.

Compound (I) can be formulated with a pharmaceutically acceptable carrier and administered orally or non-orally in the form of solid preparations such as tablets, capsules, granules and powders; or liquid preparations such as syrups and injectable preparations.

As pharmaceutically acceptable carriers, there may be used various organic or inorganic carrier substances in common use for pharmaceutical preparations, including excipients, lubricants, binders, and disintegrating agents in solid preparations; solvents, dissolution aids, suspending agents, isotonizing agents, buffers, and soothing agents in liquid preparations. Such pharmaceutical additives as antiseptics, antioxidants, coloring agents, and sweetening agents can also be used as necessary.

As examples of preferable excipients, there may be mentioned, for example, lactose, sucrose, D-mannitol, starch, crystalline cellulose, and light silicic anhydride.

As examples of preferable lubricants, there may be mentioned, for example, magnesium stearate, calcium stearate, talc, and colloidal silica.

As examples of preferable binders, there may be mentioned, for example, crystalline cellulose, sucrose, D-mannitol, dextrin, hydroxypropyl cellulose, hydroxypropylmethyl cellulose, and polyvinylpyrrolidone.

As examples of preferable disintegrating agents, there may be mentioned, for example, starch, carboxymethyl cellulose, carboxymethyl cellulose calcium, crosslinked carmellose sodium, and carboxymethyl starch sodium.

As examples of preferable solvents, there may be mentioned, for example, water for injection, alcohol, propylene glycol, macrogol, sesame oil, and corn oil.

As examples of preferable dissolution aids, there may be mentioned, for example, polyethylene glycol, propylene glycol, D-mannitol, benzyl benzoate, ethanol, trisaminomethane, cholesterol, triethanolamine, sodium carbonate, and sodium citrate.

As examples of preferable suspending agents, there may be mentioned, for example, surfactants such as stearyltriethanolamine, sodium lauryl sulfate, laurylaminopropionic acid, lecithin, benzalkonium chloride, benzethonium chloride, and monostearic glycerol; and hydrophilic polymers such as polyvinyl alcohol, polyvinylpyrrolidone, carboxymethyl cellulose sodium, methyl cellulose, hydroxymethyl cellulose, hydroxyethyl cellulose, and hydroxypropyl cellulose.

As examples of preferable isotonizing agents, there may be mentioned, for example, sodium chloride, glycerol, and D-mannitol.

As examples of preferable buffers, there may be mentioned, for example, buffer solutions of phosphates, acetates, carbonates, citrates, etc.

As examples of preferable soothing agents, there may be 30 mentioned, for example, benzyl alcohol.

As examples of preferable antiseptics, there may be mentioned, for example, para-oxybenzoic acid esters, chlorobutanol, benzyl alcohol, phenethyl alcohol, dehydroacetic acid, and sorbic acid.

As examples of preferable antioxidants, there may be mentioned, for example, sulfites and ascorbic acid.

A pharmaceutical composition can be produced by a conventional method by containing the compound (I) in a ratio of normally 0.1 to 95% (w/w) to the total amount of the preparation, although the ratio varies depending on dosage form, method of administration, carrier, etc.

In addition, cancer can be more effectively prevented or treated by the combination of (1) administering an effective amount of compound (I) with (2) 1-3 kinds selected from the group consisting of (i) administering an effective amount of another anti-cancer agent, (ii) administering an effective amount of hormonotherapy agent, and (iii) non-pharmaceutical therapy. As the non-pharmaceutical therapy, for example, operation, radiation therapy, gene therapy, thermotherapy, cryotherapy, laser burning therapy and the like can be mentioned, wherein two or more kinds thereof may be used in combination.

For example, the compound of the present invention can be used simultaneously with hormonal therapeutic agents, anticancer agent (e.g., chemotherapeutic agents, immunotherapeutic agents, or drugs that inhibit the activity of growth factors or receptors)(after here, these are referred to as a combination drug).

Although the compound (I) exhibits excellent anticancer action even when used as a simple agent, its effect can be enhanced by using it in combination with one or more of the combination drugs mentioned above (multi-agent co-administration).

As examples of said “hormonal therapeutic agents,” here may be mentioned fosfestrol, diethylstilbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, anti-estrogens (e.g., tamoxifen citrate, toremifene citrate), pill preparations, mepitiostane, testrolactone, aminoglutethimide, LH-RH agonists (e.g., goserelin acetate, buserelin, leuprorelin), droloxifene, epitiostanol, ethinylestradiol sulfonate, aromatase inhibitors (e.g., fadrozole hydrochloride, anastrozole, retrozole, exemestane, vorozole, formestane), anti-androgens (e.g., flutamide, bicartamide, nilutamide), 5_{α}-reductase inhibitors (e.g., finasteride, epristeride), adrenocorticohormone drugs (e.g., dexamethasone, prednisolone, betamethasone, triamcinolone), androgen synthesis inhibitors (e.g., abiraterone), and retinoid and drugs that retard retinoid metabolism (e.g., liarozole), etc., and LH-RH agonists (e.g., goserelin acetate, buserelin, leuprorelin) are preferable.

As examples of said “chemotherapeutic agents”, there may be mentioned alkylating agents, antimetabolites, anticancer antibiotics, and plant-derived anticancer agents.

As examples of “alkylating agents”, there may be mentioned nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, umitepa, ribomustin, temozolomide, treosulphan, rophosphamide, zinostatin stimalamer, carboquone, adozelesin, ystemustine, and bizelesin.

As examples of “antimetabolites”, there may be mentioned mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, and ambamustine, etc.

As examples of “anticancer antibiotics”, there may be mentioned actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, and idarubicin hydrochloride, etc.

As examples of “plant-derived anticancer agents”, there may be mentioned etoposide, etoposide phosphate, vinblastine sulfate, vincristine sulfate, vindesine sulfate, teniposide, paclitaxel, docetaxel, vinorelbine, camptothecine, and irinotecan hydrochloride etc.

As examples of said “immunotherapeutic agents (BRM)”, there may be mentioned picibanil, krestin, sizofiran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, and procodazole.

The “growth factor” in said “drugs that inhibit the activity of growth factors or growth factor receptors”, there may-be mentioned any substances that promote cell proliferation, which are normally peptides having a molecular weight-of not more than 20,000 that are capable of exhibiting their activity at low concentrations by binding to a receptor, including (1) EGF (epidermal growth factor) or substances possessing substantially the same activity as it [e.g., EGF, heregulin (HER2 ligand), etc.], (2) insulin or substances possessing substantially the same activity as it [e.g., insulin, IGF (insulin-like growth factor)-1, IGF-2, etc.], (3) FGF (fibroblast growth factor) or substances possessing substantially the same activity as it [e.g., acidic FGF, basic FGF, KGF (keratinocyte growth factor), FGF-10, etc.], and (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), etc.].

As examples of said “growth factor receptors”, there may be mentioned any receptors capable of binding to the aforementioned growth factors, including EGF receptor, heregulin receptor (HER2), insulin receptor, IGF receptor, FGF receptor-1 or FGF receptor-2, and the like.

As examples of said “drugs that inhibit the activity of cell growth factor”, there may be mentioned trastuzumab (Herceptin (trade mark); anti-HER2 antibody), ZD1839 (IRESSA), and GLEEVEC.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan), topoisomerase II inhibitors (e.g., sobuzoxane), differentiation inducers (e.g., retinoid, vitamin D), angiogenesis inhibitors, α-blockers (e.g., tamsulosin hydrochloride), etc. can be used.

Among those mentioned above, LH-RH agonists (e.g., goserelin acetate, buserelin, leuprorelin), trastuzumab (HER2 antibody), etc. are preferable as combination drugs.

In combination of the compound (I) and the combination drug, the administration time of the compound (I) and the combination drug is not restricted, and the compound (I) or the combination drug can be administered to an administration subject simultaneously, or may be administered at different times. The dosage of the combination drug may be determined according to the dose clinically used, and can be appropriately selected depending on the administration subject, administration route, disease, combination and the like.

The administration mode of the compound (I) and the combination drug is not particularly restricted, and it is sufficient that the compound (I) and the combination drug are combined in administration. Examples of such administration mode include the following methods:

(1) The compound (I) and the combination drug are simultaneously produced to give a single preparation which is administered. (2) The compound (I) and the combination drug are separately produced to give two kinds of preparations which are administered simultaneously by the same administration route. (3) The compound (I) and the combination drug are separately produced to give two kinds of preparations which are administered by the same administration route only at the different times. (4) The compound (I) and the combination drug are separately produced to give two kinds of preparations which are administered simultaneously by the different administration routes. (5) The compound (I) and the combination drug are separately produced to give two kinds of reparations which are administered by the different administration routes only at different times (for example, the compound (I) and the combination drug are administered in this order, or in the reverse order).

In addition, the method of the present invention can be combined with a non-drug therapy such as (1) surgery, (2) hypertensive chemotherapy using angiotensin II etc., (3) gene therapy, (4) immunotherapy (5) thermotherapy, (6) cryotherapy, (7) laser cauterization, (8) radiotherapy, etc.

For example, the method of the present invention provides benefits such as inhibition of resistance expression, extend disease-free survival, suppresses cancer metastasis or recurrence, prolongs survival when used before or after the surgery, etc., or a combination treatment comprising 2 or 3 of these therapies.

Also, treatment with the method of the present invention can be combined with supportive therapies [e.g., (i) administration of antibiotics (e.g., β-lactams such as pansporin™, macrolides such as clarytheromycin™) to an coincidence of various infectious diseases, (ii) intravenous hyperalimentation, administration of amino acid preparations and general vitamin preparations for improvement of malnutrition, (iii) morphine administration for pain mitigation, (iv) administration of drugs which mitigate adverse reactions such as nausea, vomiting, anorexia, diarrhea, leukopenia, thrombocytopenia, hemoglobin concentration reduction, hair loss, hepatopathy, nephrophathy, DIC and fever, (v) administration of drugs for inhibition of multiple drug resistance in cancer].

It is preferable to practice the method of the present invention before or after the aforementioned treatments.

As a period for treatment with the method of the present invention before the surgery, etc., for example, it can be performed one time about 30 minutes to 24 hours before the surgery, etc., or in 1 to 3 cycles about 3 months to 6 months before the surgery, etc. In this way, the surgery, etc. can be conducted easily because, for example, a cancer tissue would be reduced by treatment with the method of the present invention before the surgery, etc.

As a period for treatment with the method of the present invention after the surgery, etc., for example, it can be conducted repeatedly per a few weeks to 3 months, about 30 minutes to 24 hours after the surgery, etc. In this way, it increases the effectiveness of the surgery by conducting the method of the present invention after the surgery, etc.

While the dose of the inhibitor (particularly trastuzumab) of an ErbB-2 (HER2) extracellular moiety varies depending on the administration route, condition and the like, when, for example, it is intravenously administered as an anticancer agent to patients (body weight 40-80 kg) with breast cancer or prostate cancer, it is, for example, 0.5-100 mg/kg body weight/week, preferably 1-10 mg/kg body weight/week, more preferably 1-5 mg/kg body weight/week.

### EXAMPLES

While the present invention is explained in detail in the following, the present invention is not limited thereby.

In the Reference Examples, elution in column chromatography was conducted under observation by TLC (Thin Layer Chromatography). In TLC observation, the TLC plate used was the Merck Kieselgel 60F₂₅₄ plate, the developing solvent used was the solvent used as the eluent for column, and the means of detection used was an UV detector. The silica gel for the column was also Merck Kieselgel 60F₂₅₄ (70-230 mesh). NMR spectra are shown by proton NMR with tetramethylsilane as the internal standard, using VARIAN Gemini-206 (200 MHz type spectrometer); δ values are expressed in ppm.

The abbreviations used in the Reference Examples are defined as follows:

s: Singlet

br: Broad

d: Doublet

t: Triplet

q: Quartet

dd: Double doublet

dt: Double triplet

m: Multiplet

J: Coupling constant

Hz: Hertz

DMF: N,N-dimethylformamide

THF: Tetrahydrofuran

### Reference Example A11

4-chloromethyl-2-[(E)-2-(4-methylphenyl)ethenyl]-1,3-oxazole

(i) (E)-3-(4-methylphenyl)-2-propenamide

To a solution of 4-methylcinnamic acid (15.19 g) in THF (100 mL), DMF (5 drops) was added; under ice cooling, oxalyl chloride (9.6 mL) was added, followed by stirring at room temperature for 2 hours. After oxalyl chloride (4.0 mL) was added, the reaction mixture was stirred at room temperature for 1 hour, after which it was concentrated to dryness. The residue was dissolved in ethyl acetate (50 mL); under ice cooling, this solution was added drop by drop to a mixture of 25% aqueous ammonia (50 mL)-ethyl acetate (20 mL). The water layer was salted out; the organic layer was extracted with ethyl acetate. The extract was dried over magnesium sulfate, after which it was concentrated under reduced pressure. The precipitate was washed with hexane and diethyl ether to yield the titled compound (11.63 g) as colorless crystals.

¹H-NMR(CDCl₃) δ: 2.37 (3H,s), 5.56(2H,brs), 6.41(1H,d,J=15.8), 7.18(2H,d,J=8.0), 7.42(2H,d,J=8.0), 7.62(1H,d,J=15.8).

IR (KBr): 1671, 1601, 1518, 1397, 1254, 1123, 990, 816 cm⁻¹.

(ii) 4-chloromethyl-2-[(E)-2-(4-methylphenyl)ethenyl]-1,3-oxazole

A mixture of (E)-3-(4-methylphenyl)-2-propenamide (8.06 g) and 1,3-dichloroacetone (6.98 g) in toluene (50 mL) was refluxed for 3 hours. After cooling, the reaction mixture was diluted with ethyl acetate, washed with water and saline, and dried over magnesium sulfate, then concentrated under reduced pressure. The residue was purified by silica gel column chromatography (eluent:ethyl acetate-hexane 1:4) to yield the titled compound (8.44 g) as a white crystalline powder.

¹H-NMR(CDCl₃)δ: 2.38 (3H,s), 4.54 (2H,s), 6.87 (1H,d,J=16.2), 7.20 (2H,d,J=8.2), 7.43 (2H,d,J=8.2), 7.52 (1H,d,J=16.2), 7.62 (1H,s).

IR (KBr): 1642, 1607, 1591, 1537, 1345, 1267, 976, 943, 810 cm⁻¹.

### Reference Example A2

4-chloromethyl-2-[(E)-2-(4-fluorophenyl)ethenyl]-1,3-oxazole

4-Fluorocinnamic acid (25 g) was suspended in dichloromethane (300 mL); under ice cooling and stirring, DMF (0.5 mL) and then oxalyl chloride (15.36 mL) were added drop by drop; the same temperature was kept for 3 hours and gradually returned to room temperature. Under reduced pressure, the solvent was distilled off; the residue was dissolved in ethyl acetate (100 mL). This solution was added drop by drop to an ice-cooled mixed solution of 25% aqueous ammonia (250 mL) and ethyl acetate (52.5 mL). The reaction mixture was extracted with ethyl acetate (400 mL×2) and washed with saturated saline, after which it was dried over anhydrous magnesium sulfate. Under reduced pressure, the solvent was distilled off; the precipitated crystals were collected by filtration and dried to yield (E)-3-(4-fluorophenyl)-2-propenamide (24.4 g).

The (E)-3-(4-fluorophenyl)-2-propenamide (17.55 g) thus obtained and 1,3-dichloroacetone (12.85 g) were molten at 130° C. and stirred for 1.5 hours. After the reaction mixture was cooled to room temperature and extracted with ethyl acetate, it was washed with ice water, saturated aqueous sodium bicarbonate, and saturated saline. After drying with anhydrous sodium sulfate, the solvent was distilled off; the residue was purified by column chromatography (eluent: diethyl ether-hexane=1:9-+3:17) to yield the titled compound (10.5 g) as colorless crystals.

¹H-NMR(CDCl₃)δ: 4.54 (2H,s), 6.84 (1H,d,J=16.0 Hz) 7.09 (2H,t,J=8.8 Hz), 7.47-7.55 (3H,m), 7.63 (1H,s).

IR (KBr): 3173, 3133, 3063, 3040, 1645, 1601, 1591, 1537, 1508, 1435, 1416, 1350, 1275, 1233, 1167, 1101, 999 cm⁻¹.

### Reference Example A3

4-chloromethyl-2-[(E)-2-(4-trifluoromethylphenyl)ethenyl]-1,3-oxazole

(i) (E)-3-(4-trifluoromethylphenyl)-2-propenamide

To a suspension of 4-trifluoromethylcinnamic acid (19.4 g) and DMF (6 drops) in THF (100 mL), oxalyl chloride (11.7 mL) was added drop by drop at 0° C., followed by stirring at room temperature for 2 hours. After the solvent was distilled off under reduced pressure, the residue was dissolved in ethyl acetate (60 mL) and poured into a mixture of 25% aqueous ammonia-ethyl acetate (5:1, 120 mL). After salting-out, the water layer was extracted with a mixture of ethyl acetate-THF (12:1) (650 mL) and ethyl acetate (100 mL×2) and dried over anhydrous magnesium sulfate. After the solvent was distilled off under reduced pressure, the residue was recrystallized from ethyl acetate-hexane to yield the titled compound (18.0 g) as colorless tabular crystals.

¹H-NMR (CDCl₃) δ: 5.58 (2H, br s), 6.53 (1H, d, J=15.8 Hz), 7.63-7.72 (5H, m).

IR (KBr): 3326, 3167, 1686, 1636, 1617, 1404, 1190 cm⁻¹.

(ii) 4-chloromethyl-2-[(E)-2-(4-trifluoromethylphenyl)ethenyl]-1,3-oxazole

A solution of (E)-3-(4-trifluoromethylphenyl)-2-propenamide (17.9 g) and 1,3-dichloroacetone (14.8 g) in toluene (83 mL) was refluxed under heating for 9 hours using a Dean-Stark apparatus. After cooling, water was added; the reaction mixture was extracted with ethyl acetate and washed with saturated saline, after which it was dried over anhydrous magnesium sulfate. After the solvent was distilled off under reduced pressure, the residue was purified by silica gel column chromatography (eluent: hexane-methyl acetate=6:1→5:1) to yield the titled compound (15.1 g) as colorless needle crystals.

¹H-NMR (CDCl₃) δ: 4.55 (2H, d, J=0.8 Hz), 7.00 (1H, d, J=16.2 Hz), 7.56 (1H, d, J=16.2 Hz), 7.64-7.68 (5H, m).

IR (KBr): 1350, 1325, 1170, 1136, 1113, 1071, 959, 826, 727, 708 cm⁻¹.

### Reference Example A4

4-chloromethyl-2-[(E)-2-(2,4-difluorophenyl)ethenyl]-1,3-oxazole

Using (E)-3-(2,4-difluorophenyl)-2-propenamide (9.16 g) and 1,3-dichloroacetone (7.62 g), the same reaction as Reference Example A1-(ii) was carried out to yield the titled compound (6.31 g) as colorless crystals.

¹H-NMR (CDCl₃) δ: 4.55 (2H, s), 6.8-7.0 (2H, m), 6.96 (1H, d, J=16.8), 7.45-7.7 (3H, m).

### Reference Example A5

4-chloromethyl-2-[(E)-2-(2,6-difluorophenyl)ethenyl]-1,3-oxazole

Using (E)-(2,6-difluorophenyl)-2-propenamide (9.0 g) and 1,3-dichloroacetone (7.49 g), the same reaction as Reference Example A1-(ii) was carried out to yield the titled compound (7.18 g) as a light-yellow solid.

¹H-NMR (CDCl₃) δ: 4.55 (2H, s), 6.85-7.0 (2H, m), 7.2-7.35 (2H, m), 7.55-7.7 (1H, m), 7.66 (1H, s).

### Reference Example A6

3-(1H-imidazol-2-yl)-1,2-propanediol

3,4-Dihydroxybutyronitrile (30.33 g) was dissolved in absolute methanol (12.2 mL); under ice cooling and stirring, a 5.12 N solution of hydrogen chloride in ether (62 mL) was added under 5° C. The reaction mixture was stirred at constant temperature for 35 hours to yield a double-layered solution. The upper layer was removed, and the lower layer was dissolved in absolute methanol (45 mL). A solution of aminoacetaldehyde dimethylacetal (31.5 g) in absolute methanol (45 mL) was added under ice cooling and stirring under 20° C., followed by stirring for 27 hours. Under reduced pressure, the solvent was distilled off; to the residue, water (57 mL) and concentrated hydrochloric acid (142 mL) were added, followed by stirring at room temperature for 2 hours. Under reduced pressure, the solvent was distilled off; to the residue, an aqueous solution of potassium carbonate was added; after adjustment to pH 10, the solvent was again distilled off. The residue was extracted with ethanol (500 mL) and concentrated to dryness. After purification by silica gel column chromatography, the concentrated extract was desalinized with an ion exchange resin (Amberlyst 15) to yield the titled compound (13.16 g) as pale-brown crystals.

mp 98-100° C.

¹H-NMR (DMSO-d₆) δ: 2.60 (1H,dd,J=7.6 Hz, 14.8 Hz), 2.80 (1H,dd,J=5.0 Hz, 14.8 Hz), 3.28 (1H,dd,J=5.6 Hz, 10.2 Hz), 3.35 (1H,dd,J=5.4 Hz, 10.2 Hz), 3.72-3.85 (1H,m), 6.88 (2H,s).

IR (KBr): 3167, 3094, 2928, 2656, 1559, 1456, 1416, 1379, 1327, 1291, 1275, 1242, 1202, 1152, 1111, 1092, 1044 cm⁻¹.

### Reference Example A7

(2R)-3-(1H-imidazol-2-yl)-1,2-propanediol

(i) (2R)-1-(benzyloxy)-3-(1-trityl-1H-imidazol-2-yl)-2-propanol

In an argon atmosphere, n-butyllithium (1.6 M solution in hexane, 6.9 ml) was added drop by drop to a solution of 1-tritylimidazole (3.10 g) in THF (80 mL) under ice cooling. After stirring at the same temperature for 30 minutes, (R)-2-[(benzyloxy)methyl]oxirane (1.52 mL) was added. After stirring under ice cooling for 1.5 hours and at room temperature for 1 hour, water was added and the reaction mixture was extracted with ethyl acetate. The extract was washed with water and saline and dried over magnesium sulfate, after which it was concentrated under reduced pressure. The residue was purified by silica gel chromatography (eluent: ethyl acetate-hexane 1:1) to yield the titled compound (1.402 g) as a pale-yellow oily substance.

¹H-NMR (CDCl₃) δ: 2.06 (2H, dd, J=2.8 Hz, 18.0 Hz), 3.08 (1H, dd, J=5.4 Hz, 9.8 Hz), 3.21 (1H, dd, J=5.4 Hz, 9.8 Hz), 3.55-3.7 (1H, m), 4.36 (2H, s), 6.73 (1H, d, J=1.4 Hz), 6.93 (1H, d, J=1.4 Hz), 7.0-7.4 (20H, m).

(ii) (2R)-1-(benzyloxy)-3-(1H-imidazol-2-yl)-2-propanol

To a solution of (2R)-1-(benzyloxy)-3-(1-trityl-1H-imidazol-2-yl)-2-propanol (1.40 g) in acetone (8 mL), 1 N hydrochloric acid (8 mL) was added, followed by stirring at 50° C. for 1 hour. Additionally, 1 N hydrochloric acid (8 mL) was added, followed by stirring at 50° C. for 2 hours. After concentration and addition of water, the reaction mixture was twice washed with diethyl ether. After neutralization with aqueous sodium bicarbonate, the water layer was extracted with ethyl acetate and washed with saline, after which it was dried over magnesium sulfate and concentrated under reduced pressure. The residue was purified by silica gel column chromatography (eluent:ethyl acetate-methanol=10:1) to yield the titled compound (424 mg) as a colorless oily substance.

¹H-NMR (CDCl₃) δ: 2.85 (1H, dd, J=7.8 Hz, 15.6 Hz), 2.99 (1H, dd, J=3.6 Hz, 15.6 Hz), 3.39 (1H, dd, J=7.0 Hz, 9.5 Hz), 3.52 (1H, dd, J=4.4 Hz, 9.5 Hz), 4.1-4.3 (1H, m), 4.55 (2H, s), 6.94 (2H, s), 7.3-7.45 (5H, m).

(iii) (2R)-3-(1H-imidazol-2-yl)-1,2-propanediol

To a solution of (2R)-1-(benzyloxy)-3-(1H-imidazol-2-yl)-2-propanol (424 mg) in methanol (10 mL), 10% palladium carbon (50% hydrated, 85 mg) was added, followed by stirring at 50-60° C. in a hydrogen atmosphere for 2 days. The catalyst was filtered off; the filtrate was concentrated to yield the titled compound (254 mg) as a white solid.

¹H-NMR (CDCl₃) δ: 2.58 (1H,dd,J=7.6 Hz, 14.6 Hz), 2.78 (1H, dd, J=5.2 Hz, 14.6 Hz), 3.17 (1H, d, J=5.2 Hz), 3.2-3.3 (1H, m), 3.7-3.85 (1H, m), 4.6-4.7 (1H, m), 4.86 (1H, d, J=4.8 Hz), 6.76 (1H, brs), 6.95 (1H, brs).

[α]_{D}²²+2.5° (c=1.0, methanol)

### Reference Example A8

(2S)-3-(1H-imidazol-2-yl)-1,2-propanediol

(i) (3S)-4-(benzyloxy)-3-(trimethylsilyloxy)butyronitrile

To a mixture of (2S)-2-[(benzyloxy)methyl]oxirane (6.57 g) and trimethylsilanecarbonitrile (5.0 g), potassium cyanide (26 mg) and 18-crown-6 (106 mg) were added, followed by refluxing at 135° C. in an argon atmosphere for 75 minutes. After cooling, the reaction mixture was subjected to distillation under reduced pressure to yield the titled compound (7.42 g).

¹H-NMR (CDCl₃) δ: 0.15 (9H,s), 2.52 (1H,dd,J=6.6 Hz, 16.6 Hz), 2.65 (1H,dd, J=4.6 Hz, 16.6 Hz), 3.39 (1H,dd,J=6.8 Hz, 9.6 Hz), 3.50 (1H,dd,J=4.8 Hz, 9.6 Hz), 4.01-4.14 (1H,m), 4.52 (2H,s), 7.26-7.44 (5H,m).

IR (neat): 3065, 3032, 2957, 2903, 2865, 2251, 1607, 1588, 1497, 1454, 1416, 1366, 1254, 1209, 1117, 1001 cm¹.

(ii) (3S)-4-(benzyloxy)-3-hydroxybutyronitrile

(3S)-4-(Benzyloxy)-3-[(trimethylsilyl)oxy]butyronitrile (7.41 g) was dissolved in tetrahydrofuran (28.2 mL); under ice cooling and stirring, a 1 M solution of tetrabutylammonium fluoride in THF (28.2 mL) was added, followed by stirring for 1.5 hours. Under reduced pressure, the solvent was distilled off, the residue was dissolved in ether and washed with water and saturated saline. Under reduced pressure, the solvent was distilled off; the residue was purified by silica gel column chromatography to yield the titled compound (4.58 g) as a colorless oily substance.

¹H-NMR(DMSO-d₆) δ: 2.56 (1H,dd,J=6.4 Hz, 16.8 Hz), 2.70 (1H,dd,J=4.6 Hz, 16.8 Hz), 3.34 (1H,dd,J=6.2 Hz, 9.8 Hz), 3.44 (1H,dd,J=5.4 Hz, 9.8 Hz), 3.85-3.95 (1H,m), 5.52 (2H,d,J=5.2 Hz), 7.25-7.40 (5H,m).

IR (neat): 3600-3200, 3065, 3032, 2867, 2253, 1605, 1586, 1497, 1454, 1416, 1364, 1308, 1254, 1208, 1101, 1078 cm⁻¹.

(iii) (2S)-1-(benzyloxy)-3-(1H-imidazol-2-yl)-2-propanol

Using (3S)-4-(benzyloxy)-3-hydroxybutyronitrile (6.51 g), a 5.12 N solution of hydrogen chloride in ether (7.0 mL), and aminoacetaldehyde dimethyl acetal (3.58 g), the same reaction as Reference Example 6 was carried out to yield the titled compound (2.22 g) as a light-brown oily substance.

¹H-NMR(CDCl₃) δ: 2.84 (1H,dd, J=7.8 Hz, 15.4 Hz), 2.97 (1H,dd,J=3.6 Hz, 15.4 Hz), 3.41 (1H,dd,J=6.8 Hz, 9.4 Hz), 3.51 (1H,dd,J=4.4 Hz, 9.4 Hz), 4.11-4.23 (1H,m), 4.54 (2H,s), 6.91 (2H,s), 7.27 (5H,m).

IR (neat): 3400-3140, 3065, 3032, 2903, 2865, 1601, 1557, 1495, 1454, 1427, 1366, 1312, 1206, 1101, 1028 cm⁻¹.

[α]_{D}²²=−2.3° (c=1.04, methanol)

(iv) (2S)-3-(1H-imidazol-2-yl)-1,2-propanediol

(2S)-1-(Benzyloxy)-3-(1H-imidazol-2-yl)-2-propanol (1.725 g) was dissolved in ethanol (30 mL); 10% palladium carbon (1.04 g) was added, followed by vigorous stirring in a hydrogen atmosphere at 60° C. and 5 atm for 24 hours. The catalyst was filtered off and the solvent was distilled off; the residue was purified by silica gel flush column chromatography to yield the titled compound (0.945 g).

The spectral data (¹H-NMR, IR) of this product agreed with those of the compound of Reference Example 6.

### Reference Example A9

(i) 4-(4-benzyloxyphenyl)-3-buten-1-ol

In an argon atmosphere, 3-hydroxypropyltriphenylphosphonium bromide (4.02 g) was suspended in dehydrated THF (30 mL); 60% oily sodium hydride (0.4 g) was added, followed by refluxing for 3 hours. To the reaction mixture, a solution of 4-benzyloxybenzaldehyde (2.12 g) in dehydrated THF (7 mL) was added drop by drop, followed by refluxing for 67 hours. After cooling, the insoluble matter was filtered off; the filtrate was concentrated under reduced pressure. The residue was purified by column chromatography (eluent: hexane-ethyl acetate=9:1→4:1) to yield the titled compound (1.76 g) as colorless crystals.

¹H-NMR (CDCl₃) δ: 2.46 (0.8H,dq,J=1.4 Hz, 6.2 Hz), 2.61 (1.2H,dq,J=1.6 Hz, 6.4 Hz), 3.71-3.78 (2H,m), 5.06 (1.2H,s), 5.07 (1.8H,s), 5.59 (0.6H,dt,J=7.2 Hz, 11.6 Hz), 6.07 (0.4H,dt,J=7.2 Hz, 15.8 Hz), 6.45 (0.4H,d,J=15.8 Hz), 6.52 (0.6H,d,J=11.6 Hz), 6.89-6.98 (2H,m), 7.22-7.46(7H,m).

IR (KBr): 3279, 3063, 3036, 3011, 2911, 2867, 1607, 1574, 1510, 1470, 1454, 1383, 1302, 1250, 1177, 1117, 1053, 1017 cm⁻¹.

(ii) 4-(4-hydroxybutyl)phenol

4-(4-Benzyloxyphenyl)-3-buten-1-ol (1.70 g) was dissolved in a mixture of methanol-THF (1:1, 20 mL); 10% palladium carbon (0.17 g) was added, followed by vigorous stirring in a hydrogen atmosphere for 1.5 hours. The catalyst was filtered off; the filtrate was concentrated under reduced pressure to yield the titled compound (1.1 g) as a colorless crystalline powder.

¹H-NMR(CDCl₃) δ: 1.50-1.76 (4H,m), 2.57 (2H,t,J=7.1 Hz), 3.67 (2H,t,J=6.2 Hz), 6.74 (2H,d,J=8.4 Hz), 7.03 (2H,d,J=8.4 Hz).

IR (KBr): 3500-3100, 3025, 2940, 2859, 1615, 1597, 1514, 1456, 1362, 1240, 1173, 1107, 1055, 1024 cm⁻¹.

(iii) 4-[4-(benzyloxy)phenyl]-1-butanol

In an argon atmosphere, dry DMF (115 mL) was added to 4-(4-hydroxybutyl)phenol (9.43 g) and 65% oily sodium hydride (2.4 g), followed by stirring for 15 minutes. Next, under ice cooling and stirring, a solution of benzyl bromide (9.87 g) in dry dimethylformamide (29.5 mL) was added drop by drop, followed by stirring at the same temperature for 2 hours. After ice water and a 1 N solution of potassium hydrogen sulfate were added, the reaction mixture was extracted with ethyl acetate. The organic layer was washed with saturated saline, after which it was dried with anhydrous sodium sulfate. The solvent was distilled off under reduced pressure; the residue was purified by silica gel column chromatography to yield the titled compound (10.67 g) as a colorless crystalline powder.

¹H-NMR(DMSO-d₆) δ: 1.34-1.64 (4H,m), 2.50 (2H,t, J=7.0 Hz), 3.39 (2H,dt,J=5.2 Hz, 6.4 Hz), 4.34 (1H,t,J=5.2 Hz), 5.05 (2H,s), 6.90 (2H,d,J=8.6 Hz), 7.09 (2H,d,J=8.6 Hz), 7.28-7.47 (5H,m).

IR (KBr): 3500-3200, 3048, 3036, 2928, 2907, 2861, 2840, 1615, 1582, 1514, 1472, 1454, 1379, 1360, 1298, 1285, 1250, 1175, 1119, 1063, 1012 cm⁻¹.

(iv) 4-[4-(benzyloxy)phenyl]butyl methanesulfonate

To a solution of 4-(4-benzyloxyphenyl)butanol (10 g) in ethyl acetate (390 mL), triethylamine (8.16 mL) and ethanesulfonyl chloride (4.53 mL) were added drop by drop under ice cooling. After stirring at the same temperature for 30 minutes and at room temperature for 1 hour, the reaction mixture was washed with ice water and saturated saline. After drying over anhydrous sodium sulfate, the solvent was distilled off under reduced pressure to yield the titled compound (14 g) as an oily substance. This product was used for the next process without purification.

H-NMR(CDCl₃) 8:1.64-1.86 (4H,m), 2.60 (2H,t,J=7.1 Hz), 2.98 (3H,s), 4.23 (2H,t,J=6.1 Hz), 5.05 (2H,s), 6.91 (2H,d,J=8.8 Hz), 7.09 (2H,d,J=8.8 Hz), 7.32-7.48 (5H,m).

IR (neat):3063, 3031, 2940, 2865, 1611, 1584, 1512, 1456, 1354, 1337, 1240, 1175, 1115, 1015 cm¹.

(v) benzyl 4-(4-iodobutyl)phenyl Ether

Sodium iodide (29.25 g) was dissolved in acetone (195 mL); 4-[4-(benzyloxy)phenyl]butyl methanesulfonate (13 g) was added, followed by refluxing at 80° C. for 1.5 hours. After cooling, the solvent was distilled off; to the residue, ethyl acetate (750 mL) was added; the mixture was washed sequentially with water, an aqueous solution of sodium thiosulfate, and saturated saline. The organic layer was dried over anhydrous magnesium sulfate; the solvent was distilled off under reduced pressure to yield the titled compound (14.29 g) as an oily substance. This product was used for the next process without purification.

¹H-NMR(CDCl₃) δ: 1.63-1.93 (4H,m), 2.57 (2H,t,J=7.3 Hz), 3.19 (2H,t,J=6.8 Hz), 5.04 (2H,s), 6.90 (2H,d,J=8.8 Hz), 7.09 (2H,d,J=8.8 Hz), 7.30-7.47 (5H,m).

IR (neat): 3063, 3031, 2932, 2857, 1611, 1582, 1510, 1454, 1381, 1298, 1238, 1175, 1121, 1026 cm⁻¹.

(vi) 1-[4-(4-benzyloxyphenyl)butyl]-1H-1,2,3-triazole

Benzyl 4-(4-iodobutyl)phenyl ether (1.1 g), 1H-1,2,3-triazole (0.31 g), and potassium carbonate (0.622 g) were suspended in DMF (7.5 mL), followed by stirring at 70° C. for 26.5 hours. After cooling, the reaction mixture was extracted with ethyl acetate and washed with water and saturated saline. Under reduced pressure, the solvent was distilled off; the residue was subjected to silica gel column chromatography (eluent: hexane-ethyl acetate=4:1→2:3) to yield the titled compound (0.391 g).

¹H-NMR (CDCl₃) δ: 1.61 (2H,quintet,J=7.8 Hz), 1.93 (2H,quintet,J=7.8 Hz), 2.59 (2H,t,J=7.6 Hz), 4.39 (2H,t,J=7.1 Hz), 5.04 (2H,s), 6.90 (2H,d,J=8.8 Hz), 7.06 (2H,d,J=8.8 Hz), 7.30-7.48 (5H,m), 7.49 (1H,s), 7.69 (1H,s).

IR (KBr):3106, 3034, 2940, 2861, 1611, 1582, 1512, 1454, 1387, 1298, 1244, 1177, 1113, 1080, 1040, 1028 cm⁻¹.

(vii) 4-[4-(1H-1,2,3-triazol-1-yl)butyl]phenol

1-[4-(4-Benzyloxyphenyl)butyl]-1H-1,2,3-triazole (0.38 g) was dissolved in methanol (7.6 mL); 10% palladium carbon (0.1 g) was added, followed by vigorous stirring in a hydrogen atmosphere for 14 hours. The catalyst was filtered off; the filtrate was concentrated to dryness under reduced pressure to yield the titled compound (0.268 g) as a crystalline powder.

¹H-NMR (CDCl₃) δ: 1.60 (2H,quintet,J=7.0 Hz), 1.93 (2H,quintet,J=7.4 Hz), 2.57 (2H,t,J=7.5 Hz), 4.40 (2H,t,J=7.0 Hz), 6.79 (2H,d,J=8.6 Hz), 6.99 (2H,d,J=8.6 Hz), 7.51 (1H,s), 7.71 (1H,s).

IR (KBr): 3148, 3129, 3017, 2946, 2861, 2814, 1615, 1593, 1514, 1462, 1381, 1269, 1242, 1225, 1123, 1078 cm⁻¹.

### Reference Example A10

4-[3-(1H-1,2,3-triazol-1-yl)propyl]phenol

Benzyl 4-(3-iodopropyl)phenyl ether (2.47 g), 1H-1,2,3-triazole (629 mg), and potassium carbonate (1.26 g) were suspended in DMF (17.5 mL), followed by stirring at 70° C. for 18.5 hours. The reaction mixture was returned to room temperature and extracted with ethyl acetate, after which it was washed with water and saturated saline. Under reduced pressure, the solvent was distilled off; the residue was purified by silica gel column chromatography (eluent: hexane-ethyl acetate=4:1→2:3) to yield 1-[3-(4-benzyloxyphenyl)propyl]-1H-1,2,3-triazole (856 mg).

¹H-NMR (CDCl₃) δ: 2.23 (2H,quintet,J=7.2 Hz), 2.60 (2H,t,J=7.5 Hz), 4.38 (2H,t,J=7.1 Hz), 5.05 (2H,s), 6.92 (2H,d,J=8.8 Hz), 7.10 (2H,d,J=8.8 Hz), 7.30-7.48 (5H,m), 7.52 (1H,s), 7.72 (1H,s).

IR (KBr): 3100, 3030, 2960, 2926, 2860, 1613, 1585, 1514, 1454, 1383, 1298, 1250, 1215, 1177, 1115, 1082, 1044, 1028, 1019 cm⁻¹.

1-[3-(4-Benzyloxyphenyl)propyl]-1H-1,2,3-triazole (850 mg) was dissolved in methanol (29 mL); 10% palladium carbon (0.1 g) was added, followed by vigorous stirring in a hydrogen atmosphere for 13 hours. The catalyst was filtered off; the filtrate was concentrated to dryness under reduced pressure to yield the titled compound (600 mg) as a crystalline powder.

¹H-NMR(CDCl₃) δ: 2.22 (2H,quintet,J=7.0 Hz), 2.56 (2H,t,J=7.0 Hz), 4.38 (2H,t,J=7.0 Hz), 6.87 (2H,d,J=8.6 Hz), 7.04 (2H,d,J=8.6 Hz), 7.55 (1H,s), 7.74 (1H,s).

IR (KBr):3127, 3100, 3015, 2932, 1615, 1595, 1516, 1456, 1373, 1244, 1223, 1175, 1121, 1080, 1038 cm⁻¹.

### Reference Example A11

3-[3-(1H-1,2,3-triazol-1-yl)propyl]phenol

(i) 3-[3-(benzyloxy)phenyl]-1-propanol

In an argon stream, 3-benzyloxybenzaldehyde (21.3 g) and ethyl diethylphosphonoacetate (23.6 g) were suspended in dry DMF (250 mL). Under ice cooling and stirring, 65% oily sodium hydride (3.88 g) was added little by little; after completion of this addition, the mixture was stirred at room temperature for 2 hours. After the solvent was distilled off, the residue was dissolved in ethyl acetate and washed with water and saturated saline, after which it was dried over anhydrous sodium sulfate. Under reduced pressure, the solvent was distilled off to yield 33.15 g of a crude product of ethyl (E)-3-[3-(benzyloxy)phenyl]-2-propenate as an oily substance. This product was dissolved in ethanol (406 mL); ethylenediamine-treated 5% palladium carbon [Pd-C (en), 2.7 g] was added, followed by vigorous stirring in a hydrogen atmosphere. Hydrogen (1.75 L) was consumed to complete hydrogenation, and the catalyst was filtered off. Under reduced pressure, the solvent was distilled off; the residue was dissolved in dehydrated THF (120 mL). This solution was added drop by drop to a mixture of lithium aluminum hydride (4.61 g) suspended in dehydrated THF (120 mL) mixture under ice cooling. The reaction mixture was stirred under ice cooling for 1.5 hours and at room temperature for 1 hour. The reaction mixture was added to ice water and acidified, after which it was extracted with ethyl acetate, washed with water and saturated saline, after which it was dried over anhydrous sodium sulfate. Under reduced pressure, the solvent was distilled off; the residue was purified by silica gel column chromatography to yield the titled compound (14.39 g) as a colorless oily substance.

¹H-NMR (CDCl₃) δ: 1.80-1.96 (2H,m), 2.69 (2H,t,J=7.7 Hz), 3.66 (2H,t,J=6.4 Hz), 5.05 (2H,s), 6.77-6.87 (3H,m), 7.20 (1H,t,J=8.0 Hz), 7.28-7.48 (5H,m).

IR (neat): 3330, 3063, 3032, 2940, 2867, 1599, 1582, 1487, 1453, 1381, 1314, 1258, 1155, 1026 cm⁻¹.

(ii) 3-[3-(benzyloxy)phenyl]propyl Methanesulfonate

Using 3-(3-benzyloxyphenyl)propanol (13.5 g), triethylamine (8.16 mL) and methanesulfonyl chloride (4.53 mL), the same reaction as Reference Example A9-(iv) was carried out to yield the titled compound (19.7 g) as an oily substance.

¹H-NMR (CDCl₃) δ: 2.00-2.15 (2H,m), 2.73 (2H,t,J=7.5 Hz), 2.98 (3H,s), 4.22 (2H,t,J=6.3 Hz), 5.06 (2H,s), 6.77-6.88 (3H,m), 7.22 (1H,t,J=7.7 Hz), 7.31-7.48 (5H,m).

IR (neat): 3032, 2940, 2870, 1599, 1584, 1487, 1453, 1381, 1354, 1260, 1175, 1026 cm⁻¹.

(iii) benzyl 3-(3-iodopropyl)phenyl Ether

Using 3-[3-(benzyloxy)phenyl]propyl methanesulfonate (19.7 g) and sodium iodide (29.25 g), the same reaction as Reference Example A9-(v) was carried out to yield the titled compound (18.4 g) as an oily substance.

¹H-NMR(CDCl₃) δ: 2.11 (2H,quintet,J=7.3 Hz), 2.70 (2H,t,J=7.2 Hz), 3.16 (2H,t,J=6.8 Hz), 5.06 (2H,s), 6.78-6.87 (3H,m), 7.21 (1H,t,J=7.2 Hz), 7.32-7.48 (5H,m).

IR (neat): 3063, 3031, 2934, 2861, 1599, 1582, 1487, 1451, 1381, 1316, 1258, 1213, 1155, 1080, 1028 cm⁻¹.

(iv) 1-[3-(3-benzyloxyphenyl)propyl]-1H-1,2,3-triazole

In an argon atmosphere, 1H-1,2,3-triazole (0.9 g) was dissolved in DMF (20 mL); 65% oily sodium hydride (0.48 g) was added. After stirring for 30 minutes, a solution of benzyl 3-(3-iodopropyl)phenyl ether (3.53 g) in DMF (5 mL) was added, followed by stirring at room temperature for 19 hours. The reaction mixture was diluted with ethyl acetate and washed with water and saturated saline. Under reduced pressure, the solvent was distilled off; the residue was subjected to column chromatography to yield the titled compound (1.1 g) as colorless crystals.

mp 74-75° C.

¹H-NMR (CDCl₃) δ: 2.25 (2H,quintet,J=7.2 Hz), 2.63 (2H,t, J=7.3 Hz), 4.37 (2H,t,J=7.1 Hz), 5.05 (2H,s), 6.75-6.88 (3H,m), 7.23 (1H,t,J=8.2 Hz), 7.31-7.47 (5H,m), 7.49 (1H,d,J=1.0 Hz), 7.71 (1H,d,J=1.0 Hz).

IR (KBr): 3125, 3063, 3032, 2944, 2867, 1599, 1584, 1487, 1453, 1381, 1316, 1260, 1215, 1157, 1113, 1074, 1028 cm⁻¹.

(v) 3-[3-(1H-1,2,3-triazol-1-yl)propyl]phenol

To a solution of 1-[3-(3-benzyloxyphenyl)propyl]-1H-1,2,3-triazole (0.937 g) in methanol (32 mL), 10% palladium carbon (0.1 g) was added, followed by vigorous stirring in a hydrogen atmosphere at room temperature for 8 hours. The catalyst was filtered off; the filtrate was concentrated to dryness under reduced pressure to yield the titled compound (0.593 g) as colorless crystals.

mp 85-86° C.

¹H-NMR (CDCl₃) δ: 2.24 (2H,quintet,J=7.1 Hz), 2.60 (2H,t,J=7.5 Hz), 4.38 (2H,t,J=7.1 Hz), 6.68-6.79 (3H,m), 6.96 (1H,s), 7.16 (1H,t,J=8.1 Hz), 7.54 (1H,d,J=1.0 Hz), 7.73 (1H,d,J=1.0 Hz).

IR (KBr): 3129, 3077, 3054, 2949, 2863, 2722, 2614, 1599, 1588, 1483, 1458, 1362, 1337, 1281, 1221, 1157, 1121, 1080, 1038 cm⁻¹.

### Reference Example A12

4-{4-[2-(2-hydroxyethyl)-1H-imidazol-1-yl]butyl}phenol

(i) 2-(1-{4-[4-(benzyloxy)phenyl]butyl}-1H-imidazol-2-yl)-1-ethanol

Benzyl 4-(4-iodobutyl)phenyl ether (14.29 g), 2-(2-hydroxyethyl)imidazole (13.1 g) and potassium carbonate (5.39 g) were stirred in DMF (390 mL) at 60° C. for 16 hours. After cooling, the insoluble matter was filtered off; the filtrate was concentrated under reduced pressure. The residue was dissolved in ethyl acetate and washed with water and saturated saline. Under reduced pressure, the solvent was distilled off; the residue was purified by column chromatography (eluent: ethyl acetate-methanol=19:1→9:1). The eluate was recrystallized from ethyl acetate-methanol to yield the titled compound (10.99 g) as colorless crystals.

mp 75-77° C.

¹H-NMR(CDCl₃) δ: 1.53-1.82 (4H,m), 2.58 (2H,t,J=7.1 Hz), 2.78 (2H,t,J=5.5 Hz), 3.81 (2H,t,J=6.9 Hz), 4.03 (2H,t,J=5.5 Hz), 5.04 (2H,s), 6.80 (1H,d,J=1.2 Hz), 6.90 (2H,d,J=8.6 Hz), 6.93 (1H,d,J=1.2 Hz), 7.05 (2H,d,J=8.6 Hz), 7.34-7.47 (5H,m).

IR (KBr): 3144, 3032, 2934, 2859, 1611, 1582, 1514, 1495, 1456, 1431, 1381, 1298, 1273, 1244, 1175, 1150, 1121, 1109, 1051, 1026 cm⁻¹.

(ii) 4-{4-[2-(2-hydroxyethyl)-1H-imidazol-1-yl]butyl}phenol

Using 2-(1-{4-[4-(benzyloxy)phenyl]butyl}-1H-imidazol-2-yl)-1-ethanol (10.67 g) and 10% palladium carbon (1.6 g), the same reaction as Reference Example A11-(v) was carried out to yield the titled compound (5.3 g).

mp 118-119° C.

¹H-NMR(CDCl₃) δ: 1.50-1.80 (4H,m), 2.55 (2H,t,J=7.0 Hz), 2.79 (2H,t,J=5.8 Hz), 3.82 (2H,t,J=7.0 Hz), 3.97 (2H,t,J=5.8 Hz), 3.85-4.40 (1H,br), 6.77 (2H,d,J=8.4 Hz), 6.80 (1H,s), 6.94 (1H,s), 6.96 (2H,d,J=8.4 Hz).

IR (KBr): 3600-2400, 1615, 1593, 1516, 1489, 1456, 1373, 1252, 1171, 1150, 1125, 1103, 1055 cm⁻¹.

### Reference Example A13

(i) 2-(1-{3-[4-(benzyloxy)phenyl]propyl}-1H-imidazol-2-yl)-1-1-ethanol

Using benzyl 4-(3-iodopropyl)phenyl ether (5.28 g), 2-(2-hydroxyethyl)imidazole (5.05 g) and potassium carbonate. (2.07 g), the same reaction as Reference Example A12-(i) was carried out to yield the titled compound (2.78 g) as colorless crystals.

mp 80-82° C.

¹H-NMR(CDCl₃) δ: 2.03 (2H,quintet,J=7.4 Hz), 2.58 (2H,t,J=7.4 Hz), 2.74 (2H,t,J=5.6 Hz), 3.82 (2H,t,J=7.4 Hz), 4.01 (2H,t,J=5.6 Hz), 5.05 (2H,s), 6.83 (1H,s), 6.92 (2H,d,J=8.6 Hz), 6.94 (1H,s), 7.07 (2H,d,J=8.6 Hz), 7.32-7.47 (5H,m).

IR (KBr): 3500-3100, 3110, 3063, 3032, 2934, 2865, 1611, 1584, 1512, 1495, 1454, 1381, 1298, 1240, 1177, 1152, 1121, 1057, 1024 cm⁻¹.

(ii) 4-{3-[2-(2-hydroxyethyl)-1H-imidazol-1-yl]propyl}phenol

Using 2-(1-{3-[4-(benzyloxy)phenyl]propyl}-1H-imidazol-2-yl)-1-ethanol (2.53 g) and 10% palladium carbon (0.38 g), the same reaction as Reference Example A11-(v) was carried out to yield the titled compound (1.85 g) as colorless crystals.

mp 116-117° C.

¹H-NMR(CDCl₃+CD₃OD) δ: 2.03 (2H,quintet, J=7.3 Hz), 2.55 (2H,t,J=7.3 Hz), 2.75 (2H,t, J=6.2 Hz), 3.83 (2H,t,J=7.3 Hz), 3.91 (2H,t,J=6.2 Hz), 6.77 (2H,d,J=8.6 Hz), 6.84 (1H,d, J=1.2 Hz), 6.93 (1H,d,J=1.2 Hz), 6.97 (2H,d,J=8.6 Hz).

IR (KBr): 3500-3100, 3119, 2934, 2861, 1615, 1593, 1516, 1495, 1454, 1373, 1252, 1173, 1152, 1123, 1053 cm⁻¹.

### Reference Example A14

3-{3-[2-(2-hydroxyethyl)-1H-imidazol-1-yl]propyl}phenol

(i) 2-(1-{3-[3-(benzyloxy)phenyl]propyl}-1H-imidazol-2-yl)-1-ethanol

Using benzyl 3-(3-iodopropyl)phenyl ether (3.53 g), 2-(2-hydroxyethyl)imidazole (1.46 g) and 65% oily sodium hydride (0.48 g), the same reaction as Reference Example A11-(iv) was carried out to yield the titled compound (2.66 g) as a colorless oily substance. ¹H-NMR(CDCl₃) δ: 2.05 (2H,quintet,J=7.3 Hz), 2.61 (2H,t,J=7.5 Hz), 2.73 (2H,t,J=5.5 Hz), 3.81 (2H,t,J=7.3 Hz), 4.02 (2H,t,J=5.5 Hz), 5.06 (2H,s), 6.73-6.88 (3H,m), 6.82 (1H,d,J=1.2 Hz), 6.95 (1H,d,J=1.2 Hz), 7.23 (1H,t,J=8.2 Hz), 7.31-7.48 (5H,m).

IR (neat): 3500-3100, 3067, 3034, 2938, 2867, 1599, 1584, 1524, 1491, 1453, 1381, 1316, 1260, 1155, 1119, 1053, 1026 cm⁻¹.

(ii) 3-{3-[2-(2-hydroxyethyl)-1H-imidazol-1-yl]propyl}phenol

Using 2-(1-{3-[3-(benzyloxy)phenyl]propyl}-1H-imidazol-2-yl)-1-ethanol (2.42 g) and 10% palladium carbon (0.24 g), the same reaction as Reference Example A11-(v) was carried out to yield the titled compound (1.69 g) as colorless crystals.

mp 111-113° C.

¹H-NMR(CDCl₃) δ: 2.07 (2H,quintet,J=6.9 Hz), 2.55 (2H,t,J=7.3 Hz), 2.73 (2H,t,J=5.9 Hz), 3.80 (2H,t,J=7.1 Hz), 4.00 (2H,t,J=5.9 Hz), 6.55-6.76 (3H,m), 6.86 (1H,d,J=1.4 Hz), 6.96 (1H,d,J=1.4 Hz), 7.15 (1H,t,J=7.8 Hz).

IR (KBr): 3500-3100, 3046, 2940, 2865, 2712, 2604, 1599, 1588, 1528, 1483, 1456, 1372, 1279, 1250, 1155, 1123, 1057 cm⁻¹.

### Reference Example A15

3-{1-[4-(4-hydroxyphenyl)butyl]-1H-imidazol-2-yl}-1,2-propanediol

(i) 3-{1-[4-(4-benzyloxyphenyl)butyl]-1H-imidazol-2-yl}-1,2-propanediol

Using benzyl 4-(4-iodobutyl)phenyl ether (2.05 g), 2-(2,3-dihydroxypropyl)imidazole (1.0 g) and 65% oily sodium hydride (0.259 g), the same reaction as Reference Example A11-(iv) was carried out to yield the titled compound (1.23 g) as colorless crystals.

¹H-NMR(CDCl₃) δ: 1.52-1.83 (4H,m), 2.57 (2H,t,J=7.1 Hz), 2.78 (2H,d,J=5.2 Hz), 2.79 (1H,d,J=6.8 Hz), 3.62 (1H,dd,J=4.8 Hz, 11.2 Hz), 3.74 (1H,dd,J=4.8 Hz, 11.2 Hz), 3.82 (2H,t,J=7.1 Hz), 4.12-4.23 (1H,m), 5.04 (2H,s), 6.79 (1H,d,J=1.4 Hz), 6.90 (2H,d,J=8.6 Hz), 6.91 (1H,d,J=1.4 Hz), 7.05 (2H,d,J=8.6 Hz), 7.30-7.47 (5H,m).

IR (KBr): 3500-3200, 3065, 3030, 2932, 2861, 1611, 1582, 1510, 1495, 1454, 1379, 1296, 1275, 1240, 1177, 1150, 1123, 1080, 1026 cm⁻¹.

(ii) 3-{1-[4-(4-hydroxyphenyl)butyl]-1H-imidazol-2-yl}-1,2-propanediol

Using 3-{1-[4-(4-benzyloxyphenyl)butyl]-1H-imidazol-2-yl}-1,2-propanediol (1.22 g) and 10% palladium carbon (0.18 g), the same reaction as Reference Example A11-(v) was carried out to yield the titled compound (0.918 g) as colorless crystals.

¹H-NMR(CDCl₃+CD₃OD) δ: 1.50-1.80 (4H,m), 2.55 (2H,t,J=7.0 Hz) 2.75 (1H,d,J=7.2 Hz), 2.76 (1H,d,J=5.6 Hz), 3.49 (1H,dd,J=5.4 Hz, 11.6 Hz), 3.62 (1H,dd,J=4.2 Hz, 11.6 Hz), 3.84 (2H,t,J=7.0 Hz), 3.97-4.08 (1H,m), 6.75 (2H,d,J=8.6 Hz), 6.80 (1H,d,J=1.4 Hz), 6.89 (1H,d,J=1.4 Hz), 6.97 (2H,d,J=8.6 Hz).

IR (KBr): 3500-3100, 3011, 2936, 2859, 1613, 1595, 1516, 1489, 1456, 1372, 1360, 1252, 1171, 1150, 1125, 1101, 1030 cm⁻¹.

### Reference Example A16

(i) 3-{1-[3-(3-benzyloxyphenyl)propyl]-1H-imidazol-2-yl}-1,2-propanediol

Using benzyl 3-(3-iodopropyl)phenyl ether (1.98 g), 2-(2,3-dihydroxypropyl)imidazole (1.0 g) and 65% oily sodium hydride (0.259 g), the same reaction as Reference Example A11-(iv) was carried out to yield the titled compound (1.31 g) as a colorless oily substance.

¹H-NMR(CDCl₃) δ: 2.05 (2H,quintet,J=7.3 Hz), 2.60 (2H,t,J=7.3 Hz), 2.73 (1H,d,J=4.8 Hz), 2.74 (1H,d,J=7.2 Hz), 3.61 (1H,dd,J=4.8 Hz, 11.2 Hz), 3.74 (1H,dd,J=4.8 Hz, 11.2 Hz), 3.82 (2H,t,J=7.3 Hz), 4.12-4.23 (1H,m), 5.06 (2H,s), 6.73-6.88 (3H,m), 6.81 (1H,d,J=1.2 Hz), 6.93 (1H,d,J=1.2 Hz), 7.23 (1H,t,J=8.4 Hz), 7.31-7.48 (5H,m).

IR (neat): 3500-3200, 3063, 3032, 2934, 2865, 1599, 1584, 1526, 1489, 1454, 1381, 1316, 1260, 1155, 1123, 1082, 1028 cm⁻¹.

(ii) 3-{1-[3-(3-hydroxyphenyl)propyl]-1H-imidazol-2-yl}-1,2-propanediol

Using 3-(1-[3-(3-benzyloxyphenyl)propyl]-1H-imidazol-2-yl)-1,2-propanediol (1.30 g) and 10% palladium carbon (0.195 g), the same reaction as Reference Example A11-(v) was carried out to yield the titled compound (0.979 g) as a colorless oily substance.

¹H-NMR(CDCl₃+CD₃OD) δ: 2.07(2H,quintet,J=7.4 Hz), 2.58 (2H,t,J=7.3 Hz), 2.72 (1H,d,J=6.8 Hz), 2.72 (1H,d,J=5.8 Hz), 3.50 (1H,dd,J=5.4 Hz,11.4 Hz), 3.61 (1H,d,J=4.2 Hz, 11.4 Hz), 3.85 (2H,t,J=7.3 Hz), 3.98-4.10 (1H,m), 6.60-6.74(3H,m), 6.86 (1H,d,J=1.4 Hz), 6.92 (1H,d,J=1.4 Hz), 7.14 (1H,t,J=7.8 Hz).

IR (neat): 3500-3100, 3040, 2942, 2863, 1599, 1588, 1530, 483, 1456, 1360, 1279, 1254, 1155, 1125, 1088, 1030 cm⁻¹.

### Reference Example A17

2-[(E)-2-(2,4-difluorophenyl)ethenyl]-4-[[4-(4-iodobutyl)phenoxy]methyl]-1,3-oxazole

(i) 4-[4-[2-(E)-[2-(2,4-difluorophenyl)ethenyl]-1,3-oxazol-4-yl]methoxyphenyl]-1-butanol

To a solution of 4-(4-hydroxyphenyl)-1-butanol (1.99 g) in DMF (20 mL), 60% oily sodium hydride (528 mg) was added under ice cooling, followed by stirring at room temperature for 30 minutes. Under ice cooling, (E)-4-chloromethyl-2-[2-(2,4-difluorophenyl)ethenyl]oxazole (3.37 g) was added, followed by stirring overnight at room temperature. After water and 1 N hydrochloric acid was added, the reaction mixture was extracted with ethyl acetate. After the extract was dried over magnesium sulfate, it was concentrated under reduced pressure; the residue was recrystallized from ethyl acetate-diethyl ether-hexane to yield the titled compound (3.71 g) as colorless crystals.

mp 75-76° C.

¹H-NMR (CDCl₃) δ: 1.5-1.7 (4H, m), 2.60 (2H, t, J=6.8 Hz), 3.66 (2H, t, J=6.0 Hz), 5.02 (2H, s), 6.8-6.9 (1H, m), 6.89 (2H, d, J=8.4 Hz), 6.98 (1H, d, J=17.0 Hz), 7.11 (2H, d,J=8.4 Hz), 7.5-7.6 (1H, m), 7.59 (1H, d, J=17.0 Hz), 7.66 (1H, s).

IR (KBr): 1613, 1514, 1493, 1431, 1279, 1246, 1140, 968, 856 cm⁻¹.

(ii) 2-[(E)-2-(2,4-difluorophenyl)ethenyl]-4-[[4-(4-iodobutyl)phenoxy]methyl]-1,3-oxazole

To a solution of 4-[4-[2-(E)—[2-(2,4-difluorophenyl)ethenyl]-1,3-oxazol-4-yl]methoxyphenyl]-1-butanol (3.47 g) in THF (50 mL), triethylamine (1.37 mL) was added; under ice cooling, methanesulfonyl chloride (0.77 mL) was added, followed by stirring at room temperature for 30 minutes. After water was added, the reaction mixture was extracted with ethyl acetate; the extract was washed with saline, after which it was dried over magnesium sulfate. The solvent was distilled off; to the residue, acetone (100 mL) and sodium iodide (6.75 g) were added, followed by stirring at 40-50° C. for 2 hours. The reaction mixture was concentrated; water was added; the mixture was extracted with ethyl acetate. The extract was washed sequentially with aqueous sodium thiosulfate and saline and dried over magnesium sulfate, after which it was concentrated under reduced pressure. The precipitate was collected by filtration and washed with diethyl ether-hexane to yield the titled compound (3.55 g) as a pale-yellow powder.

¹H-NMR (CDCl₃) δ: 1.6-1.9 (4H, m), 2.5-2.7 (2H, m), 3.1-3.3 (2H, m), 5.02 (2H, s), 6.8-7.2 (6H, m), 7.5-7.75 (4H, m).

IR (KBr): 1615, 1514, 1493, 1431, 1279, 1246, 1140, 966, 856 cm⁻¹.

### Reference Example A18

2-[(E)-2-(4-bromophenyl)ethenyl]-4-[[4-(4-iodobutyl)phenoxy]methyl]-1,3-oxazole

Using 4-(4-hydroxyphenyl)-1-butanol (4.99 g) and (E)-4-chloromethyl-2-[2-(4-bromophenyl)ethenyl]oxazole (7.43 g), the same reaction as Reference Example A17-(i) was carried out to yield 4-[4-[2-(E)-[2-(4-bromophenyl)ethenyl]-1,3-oxazol-4-yl]methoxyphenyl]-1-butanol (9.70 g). Using the compound obtained (4.28 g), the same reaction as Reference Example A17-(ii) was carried out to yield the titled compound (4.47 g) as a white powder.

¹H-NMR (CDCl₃) δ: 1.65-1.95 (4H,m), 2.58 (2H,t,J=7.2 Hz), 3.20 (2H,t,J=6.8 Hz), 5.02 (2H,s), 6.92 (1 h,d,J=16.4 Hz), 6.92 (2H,d,J=8.6 Hz), 7.38 (2H,d,J=8.4 Hz), 7.47 (1H,d,J=16.4 Hz), 7.52 (2H,d,J=8.4 Hz), 7.66 (1H,s).

### Reference Example B1

[1-[4-[4-[[2-[(E)-2-(4-methylphenyl)ethenyl]-1,3-oxazol-4-yl]methoxy]phenyl]butyl]-1H-1,2,3-triazole

To a solution of 4-[4-(1H-1,2,3-triazol-1-yl)butyl]phenol (174 mg) in DMF (4 mL), 60% oily sodium hydride (35 mg) was added under ice cooling, followed by stirring at room temperature for 30 minutes. Under ice cooling, (E)-4-chloromethyl-2-[2-(4-methylphenyl)ethenyl]oxazole (206 mg) was added, followed by stirring at room temperature for 2 hours. After water was added to the reaction mixture, the precipitate was collected by filtration and washed with water. The precipitate was dissolved in a mixture of THF-ethyl acetate, and the solution was washed with water and saline, and dried over magnesium sulfate, after which it was concentrated under reduced pressure. The residue was recrystallized from ethyl acetate-hexane to yield the titled compound (281 mg) as colorless crystals.

mp 154-155° C.

¹H-NMR(CDCl₃) δ: 1.5-1.7 (2H,m), 1.85-2.05 (2H, m), 2.38 (3H, s), 2.60 (2H, t, J=7.5 Hz), 4.39 (2H, t, J=7.0 Hz), 5.01 (2H, s), 6.87 (2H, d, J=8.6 Hz), 6.9-7.0-(1H, m), 7.19 (2H, d, J=8.6 Hz), 7.19 (2H, d, J=8.0 Hz), 7.42 (2H, d, J=8.0 Hz), 7.5-7.7 (4H, m).

IR (KBr): 1640, 1607, 1530, 1514, 1464, 1339, 1256, 1211, 1053, 974, 810 cm⁻¹.

Anal. Calcd for C₂₅H₂₆N₄O₂: C, 72.44; H, 6.32; N, 13.52.

Found: C, 72.36; H, 6.49; N, 13.70.

### Reference Example B2

1-{4-[4-({2-[(E)-2-(4-fluorophenyl)ethenyl]-1,3-oxazol-4-yl}methoxy)phenyl]butyl}-1H-1,2,3-triazole

In an argon atmosphere, 4-[4-(1H-1,2,3-triazol-1-yl)butyl]phenol (218 mg) and 65% oily sodium hydride (39 mg) were dissolved in DMF (5 mL) added thereto. With stirring under ice cooling, 4-(chloromethyl)-2-[(E)-2-(4-fluorophenyl)ethenyl]-1,3-oxazole (250 mg) was added, followed by stirring at room temperature for 3 hours. After water was added, the reaction mixture was extracted with ethyl acetate. The extract was washed with water and saturated saline and dried over sodium sulfate, after which it was concentrated under reduced pressure. The residue was purified by silica gel column chromatography (eluent; chloroform:ethanol=24:1), after which it was recrystallized from ethyl acetate to yield the titled compound (368 mg) as colorless crystals.

mp 124-125° C.

H-NMR(CDCl₃) δ: 1.62 (2H,quintet,J=7.0 Hz), 1.94 (2H,quintet,J=7.5 Hz), 2.61 (2H,t,J=7.5 Hz), 4.40 (2H,t,J=7.0 Hz), 5.01 (2H,s), 6.86 (1H,d,J=16.0 Hz), 6.92 (2H,d,J=8.6 Hz), 7.08 (2H,d,J=8.6 Hz), 7.09 (2H,t,J=8.7 Hz), 7.46-7.57 (4H,m), 7.66 (1H,s), 7.70 (1H,d,J=1.0 Hz).

IR (KBr): 3420, 3160, 3120, 2940, 2924, 2865, 1644, 1599, 1584, 1532, 1512, 1466, 1435, 1400, 1337, 1302, 1248, 1229, 1211, 1177, 1161, 1113, 1076, 1049, 1030 cm⁻¹.

Anal calcd for C₂₄H₂₃N₄O₂F: C,68.88;H,5.55;N,13.39. Found: C,68.70;H,5.55;N,13.49.

### Reference Example B3

1-{3-[3-({2-[(E)-2-(4-fluorophenyl)ethenyl]-1,3-oxazol-4-yl}methoxy)phenyl]propyl}-1H-1,2,3-triazole

Using 3-[3-(1H-1,2,3-triazol-1-yl)propyl]phenol (208 mg), 65% oily sodium hydride (39 mg) and 4-(chloromethyl)-2-[(E)-2-(4-fluorophenyl)ethenyl]-1,3-oxazole (250 mg), the same reaction as Reference Example B2 was carried out to yield the titled compound (366 mg).

mp 105-0.106° C.

¹H-NMR(CDCl₃) δ: 2.26 (2H,quintet,J=7.2 Hz), 2.64 (2H,t,J=7.5 Hz), 4.39 (2H,t,J=7.0 Hz), 5.03 (2H,s), 6.78-6.89 (3H,m), 6.86 (1H,d,J=16.2 Hz), 7.09 (2H,t,J=8.6 Hz), 7.25 (1H,t,J=7.8 Hz), 7.51 (1H,d,J=16.2 Hz), 7.47-7.54 (3H,m), 7.68 (1H,s), 7.72 (1H,s).

IR (KBr): 3110, 3050, 2955, 2870, 1642, 1601, 1586, 1532, 1507, 1489, 1460, 1453, 1337, 1310, 1273, 1240, 1213, 1177, 1159, 1113, 1097, 1080, 1065 cm⁻¹.

Anal calcd for C₂₃H₂₁N₄O₂F: C,68.30;H,5.23;N,13.85.

Found: C,68.22;H,5.04;N,14.00.

### Reference Example B4

1-(4-{4-[(2-{(E)-2-[4-(trifluoromethyl)phenyl]ethenyl}-1,3-oxazol-4-yl)methoxy]phenyl}butyl)-1H-1,2,3-triazole

Using 4-[4-(1H-1,2,3-triazol-1-yl)butyl]phenol (152 mg), 65% oily sodium hydride (28 mg) and 4-(chloromethyl)-2-{(E)-2-[4-(trifluoromethyl)phenyl]ethenyl}-1,3-oxazole (212 mg), the same reaction as Reference Example B2 was carried out to yield the titled compound (290 mg).

mp 160-161° C.

¹H-NMR(CDCl₃) δ: 1.62 (2H,quintet,J=7.0 Hz), 1.94 (2H,quintet,J=7.6 Hz), 2.61 (2H,t,J=7.4 Hz), 4.40 (2H,t,J=7.4 Hz), 5.02 (2H,s), 6.92 (2H,d,J=8.6 Hz), 7.02 (1H,d,J=16.6 Hz), 7.08 (2H,d,J=8.6 Hz), 7.50 (1H,s), 7.56 (1H,d,J=16.6 Hz), 7.64 (4H,s), 7.69 (1H,s), 7.71 (1H,s).

IR (KBr): 3120, 2936, 1615, 1584, 1512, 1464, 1414, 1327, 1248, 1159, 1125, 1069 cm⁻¹.

Anal calcd for C₂₅H₂₃N₄O₂F₃: C,64.10;H,4.95;N,11.96.

Found: C,64.18;H,5.12;N,11.98.

### Reference Example B5

1-(3-{4-[(2-((E)-2-[4-(trifluoromethyl)phenyl]ethenyl)-1,3-oxazol-4-yl)methoxy]phenyl}propyl)-1H-1,2,3-triazole

Using 4-[3-(1H-1,2,3-triazol-1-yl)propyl]phenol (143 mg), 65% oily sodium hydride (28 mg) and 4-(chloromethyl)-2-{(E)-2-[4-(trifluoromethyl)phenyl]ethenyl}-1,3-oxazole (212 mg), the same reaction as Reference Example B2 was carried out to yield the titled compound (232 mg).

mp 157-158° C.

¹H-NMR(CDCl₃) δ: 2.24 (2H,quintet,J=7.2 Hz), 2.61 (2H,t,J=7.3 Hz), 4.39 (2H,t,J=7.2 Hz), 5.03 (2H,s), 6.94 (2H,d,J=8.4 Hz), 7.02 (1H,d,J=16.4 Hz), 7.11 (2H,d,J=8.4 Hz), 7.52 (1H,s), 7.56 (1H,d,J=16.4 Hz), 7.64 (4H,s), 7.69 (1H,s), 7.72 (1H,s).

IR (KBr): 3129, 3100, 2934, 1613, 1584, 1547, 1510, 1449, 1416, 1337, 1329, 1291, 1238, 1179, 1140, 1109, 1071, 1009 cm⁻¹.

Anal calcd for C₂₄H₂₁N₄O₂F₃: C,63.43;H,4.66;N,12.33.

Found: C,63.21;H,4.73;N,12.26.

### Reference Example B6

1-(3-{3-[(2-{(E)-2-[4-(trifluoromethyl)phenyl]ethenyl}-1,3-oxazol-4-yl)methoxy]phenyl}propyl)-1H-1,2,3-triazole

Using 3-[3-(1H-1,2,3-triazol-1-yl)propyl]phenol (123 mg), 65% oily sodium hydride (24 mg) and 4-(chloromethyl)-2-{(E)-2-[4-(trifluoromethyl)phenyl]ethenyl}-1,3-oxazole (183 mg), the same reaction as Reference Example B2 was carried out to yield the titled compound (248 mg).

mp 115-116° C.

¹H-NMR(CDCl₃) δ: 2.26 (2H,quintet,J=7.2 Hz), 2.64 (2H,t,J=7.2 Hz), 4.39 (2H,t,J=7.2 Hz), 5.04 (2H,s), 6.77-6.91 (3H,m), 7.01 (1H,d,J=16.6 Hz), 7.25 (1H,t,J=8.4 Hz), 7.52 (1H,s), 7.56 (1H,d,J=16.6 Hz), 7.64 (4H,s), 7.71 (2H,s).

IR (KBr): 3140, 3050, 2940, 2860, 1610, 1599, 1586, 1487, 1451, 1415, 1327, 1262, 1169, 1125, 1113, 1069, 1017 cm⁻¹.

Anal calcd for C₂₄H₂₁N₄O₂F₃: C,63.43;H,4.66;N,12.33.

Found: C,63.36;H,4.73;N,12.26.

### Reference Example B7

1-{4-[4-({2-[(E)-2-(2,4-difluorophenyl)ethenyl]-1,3-oxazol-4-yl}methoxy)phenyl]butyl}-1H-1,2,3-triazole

Using 4-[4-(1H-1,2,3-triazol-1-yl)butyl]phenol (152 mg), 65% oily sodium hydride (28 mg) and 4-(chloromethyl)-2-[(E)-2-(2,4-difluorophenyl)ethenyl]-1,3-oxazole (188 mg), the same reaction as Reference Example B2 was carried out to yield the titled compound (254 mg).

mp 115-117° C.

¹H-NMR(CDCl₃) δ: 1.62 (2H,quintet,J=7.2 Hz) 1.94 (2H,quintet,J=7.5 Hz), 2.60 (2H,t,J=7.5 Hz), 4.39 (2H,t,J=7.1 Hz), 5.01 (2H,s), 6.81-6.98 (2H,m), 6.91 (2H,d,J=8.6 Hz), 6.98 (1H,d,J=16.2 Hz), 7.07 (2H,d,J=8.6 Hz), 7.47-7.53 (1H,m), 7.50 (1H,s), 7.59 (1H,d-,J=16.2 Hz), 7.67 (1H,s), 7.70 (1H,s).

IR (KBr): 3133, 2932, 2863, 1644, 1615, 1590, 1532, 1514, 1493, 1468, 1431, 1345, 1298, 1279, 1246, 1215, 1179, 1140, 1086, 1049, 1032 cm⁻¹.

Anal calcd for C₂₄H₂₂N₄O₂F₂: C,66.05;H,5.08;N,12.84.

Found: C,66.03;H,5.00;N,13.03.

### Reference Example B8

1-{3-[3-({2-[(E)-2-(2,4-difluorophenyl)ethenyl]-1,3-oxazol-4-yl}methoxy)phenyl]propyl}-1H-1,2,3-triazole

Using 3-[3-(1H-1,2,3-triazol-1-yl)propyl]phenol (143 mg), 65% oily sodium hydride (28 mg) and 4-(chloromethyl)-2-[(E)-2-(2,4-difluorophenyl)ethenyl]-1,3-oxazole (188 mg), the same reaction as Reference Example B2 was carried out to yield the titled compound (257 mg).

mp 89-90° C.

¹H-NMR(CDCl₃) δ: 2.26 (2H,quintet,J=7.3 Hz), 2.64 (2H,t,J=7.4 Hz), 4.39 (2H,t,J=7.1 Hz), 5.03 (2H,s), 6.77-6.98 (5H,m), 6.98 (1H,d,J=16.8 Hz), 7.24 (1H,t,J=7.6 Hz), 7.47-7.60 (1H,m), 7.52 (1H,s), 7.59 (1H,d,J=16.8 Hz), 7.68 (1H,s), 7.71 (1H,s).

IR (KBr): 3127, 3071, 2934, 2868, 1644, 1615, 1599, 1534, 1495, 1453, 1433, 1354, 1273, 1215, 1159, 1142, 1090, 1028 cm⁻¹.

Anal calcd for C₂₃H₂₀N₄O₂F₂: C,65.39;H,4.77;N,13.26.

Found: C,65.32;H,4.56;N,13.34.

### Reference Example B9

[1-[4-[4-[[2-[(E)-2-(2,6-difluorophenyl)ethenyl]-1,3-oxazol-4-yl]methoxy]phenyl]butyl]-1H-1,2,3-triazole

To a solution of 4-[4-(1H-1,2,3-triazol-1-yl)butyl]phenol (217 mg) in DMF (4 mL), 65% oily sodium hydride (41 mg) was added under ice cooling. After stirring at room temperature for 30 minutes, 4-(chloromethyl)-2-[(E)-2-(2,6-difluorophenyl)ethenyl]-1,3-oxazole (281 mg) was added under ice cooling, followed by overnight stirring at room temperature. Water was added under ice cooling; the precipitate was collected by filtration and washed with water, after which it was dissolved in THF-ethyl acetate. The reaction mixture was washed with water and saline and dried over magnesium sulfate, after which it was concentrated under reduced pressure. The residue was recrystallized from ethyl acetate-hexane to yield the titled compound (348 mg) as colorless crystals.

¹H-NMR(CDCl₃) δ: 1.5-1.7 (2H, m), 1.85-2.05 (2H, m), 2.60 (2H, t, J=7.4 Hz), 4.39 (2H, t, J=7.2 Hz), 5.02 (2H, s), 6.92 (2H, d, J=8.8 Hz), 6.94 (1H, d, J=17.4 Hz), 6.85-7.35 (3H, m), 7.07 (2H, d, J=8.8 Hz), 7.61 (1H, d, J=17.4 Hz), 7.45-7.7 (3H, m).

IR (KBr): 1620, 1586, 1514, 1464, 1244, 1024, 999, 968, 783 cm¹.

Anal. Calcd for C₂₄H₂₂F₂N₄O₂: C, 66.05; H, 5.08; N, 12.84.

Found: C, 65.83; H, 5.06; N, 12.93.

### Reference Example B10

2-[1-[4-[4-[[2-[(E)-2-(4-methylphenyl)ethenyl]-1,3-oxazol-4-yl]methoxy]phenyl]butyl]-1H-imidazol-2-yl]-1-ethanol

Using 4-[4-[2-(2-hydroxyethyl)-1H-imidazol-1-yl]butyl]phenol (260 mg) and (E)-4-chloromethyl-2-[2-(4-methylphenyl)ethenyl]oxazole (257 mg), the same reaction as Reference Example B1 was carried out to yield the titled compound (331 mg) as colorless crystals.

mp 108-109° C.

¹H-NMR(CDCl₃) δ: 1.5-1.8 (4H, m), 2.38 (3H, s), 2.58 (2H, t, J=7.0 Hz), 2.79 (2H, t, J=5.6 Hz), 3.82 (2H, t, J=6.8 Hz), 4.03 (2H, t, J=5.6 Hz), 5.01 (2H, s), 6.8-6.85 (2H, m), 6.89 (1H, d, J=16.6 Hz), 6.92 (2H, d, J=8.6 Hz), 7.07 (2H, d, J=8.6 Hz), 7.19 (2H, d, J=7.8 Hz), 7.43 (2H, d, J=7.8 Hz), 7.51 (1H, d, J=16.6 Hz), 7.64 (1H, s).

IR (KBr): 1510, 1240, 1055, 806 cm⁻¹.

Anal. Calcd for C₂₈H₃₁N₃O₃: C, 73.50; H, 6.83; N, 9.18.

Found: C, 73.36; H, 6.66; N, 9.12.

### Reference Example B11

2-[1-[4-[4-[[2-[(E)-2-(3-methylphenyl)ethenyl]-1,3-oxazol-4-yl]methoxy]phenyl]butyl]-1H-imidazol-2-yl]-1-ethanol

Using 4-[4-[2-(2-hydroxyethyl)-1H-imidazol-1-yl]butyl]phenol (260 mg) and (E)-4-chloromethyl-2-[2-(3-methylphenyl)ethenyl]oxazole (257 mg), the same reaction as Reference Example B1 was carried out to yield the titled compound (290 mg) as colorless crystals.

mp 109-111° C.

¹H-NMR(CDCl₃) δ: 1.55-1.8 (4H, m), 2.38 (3H, s), 2.58 (2H, t, J=7.0 Hz), 2.78 (2H, t, J=5.6 Hz), 3.82 (2H, t, J=7.0 Hz), 4.03 (2H, t, J=5.6 Hz), 5.01 (2H, s), 6.80 (1H, d, J=1.4 Hz), 6.92 (1H, d, J=16.6 Hz), 6.92 (2H, d, J=8.8 Hz), 6.93 (1H, d, J=1.4 Hz), 7.07 (2H, d, J=8.8 Hz), 7.1-7.2 (1H, m), 7.2-7.4 (3H, m), 7.51 (1H, d, J=16.6 Hz), 7.65 (1H, s).

IR (KBr): 1514, 1460, 1250, 1051, 976, 828, 789 cm⁻¹.

Anal. Calcd for C₂₈H₃₁N₃O₃.0.2H₂O: C, 72.92; H, 6.86; N, 9.11.

Found: C, 72.71; H, 6.74; N, 8.97.

### Reference Example B12

2-[1-[4-[4-[[2-[(E)-2-(2-methylphenyl)ethenyl]-1,3-oxazol-4-yl]methoxy]phenyl]butyl]-1H-imidazol-2-yl]-1-ethanol

Using 4-[4-[2-(2-hydroxyethyl)-1H-imidazol-1-yl]butyl]phenol (153 mg) and (E)-4-chloromethyl-2-[2-(2-methylphenyl)ethenyl]oxazole (151 mg), the same reaction as Reference Example B1 was carried out to yield the titled compound (167 mg) as colorless crystals.

mp 91-93° C. (ethyl acetate-hexane).

¹H-NMR(CDCl₃) δ: 1.5-1.8 (4H, m), 2.46 (3H, s), 2.59 (2H, t, J=7.0 Hz), 2.79 (2H, t, J=5.6 Hz), 3.82 (2H, t, J=7.0 Hz), 4.03 (2H, t, J=5.6 Hz), 5.02 (2H, s), 6.8-6.9 (3H, m), 6.92 (2H, d, J=8.6 Hz), 7.07 (2H, d, J=8.6 Hz), 7.2-7.3 (3H, m), 7.55-7.65 (1H, m), 7.66 (1H, s), 7.79 (1H, d, J=16.2 Hz).

IR (KBr): 1508, 1464, 1231, 1061, 1009, 862, 752 cm⁻¹.

Anal. Calcd for C₂₈H₃₁N₃O₃.0.2H₂O: C, 72.92; H, 6.86; N, 9.11.

Found: C, 72.98; H, 6.70; N, 9.23.

### Reference Example B13

2-[1-[4-[4-[[2-[(E)-2-(4-ethylphenyl)ethenyl]-1,3-oxazol-4-yl]methoxy]phenyl]butyl]-1H-imidazol-2-yl]-1-ethanol

To a solution of 4-[4-[2-(2-hydroxyethyl)-1H-imidazol-1-yl]butyl]phenol (260 mg) in DMF (4 mL), 60% oily sodium hydride (44 mg) was added under-ice cooling. After stirring at room temperature for 30 minutes, (E)-4-chloromethyl-2-[2-(4-ethylphenyl)ethenyl]oxazole (272 mg) was added under ice cooling. After stirring overnight at room temperature, water was added under ice cooling. The precipitate was collected by filtration and washed with water. The precipitate was dissolved in ethyl acetate and dried (magnesium sulfate), after which it was concentrated under reduced pressure. The residue was recrystallized from ethyl acetate-hexane to yield the titled compound (297 mg) as colorless crystals.

mp 94-95° C.

¹H-NMR(CDCl₃) δ: 1.25 (3H, t, J=7.4 Hz), 1.5-1.85 (4H, m), 2.59 (2H, t, J=7.0 Hz), 2.67 (2H, q, J=7.4 Hz), 2.79 (2H, t, J=5.4 Hz), 3.82 (2H, t, J=7.0 Hz), 4.04 (2H, t, J=5.4), 5.01 (2H, s), 6.8-7.0 (3H, m), 6.92 (2H, d, J=8.4 Hz), 7.07 (2H, d, J=8.4 Hz), 7.2-7.3 (2H, m), 7.4-7.5 (2H, m), 7.53 (1H, d, J=17.2 Hz), 7.65 (1H, s).

IR (KBr): 1508, 1462, 1231, 1181, 1061, 1007, 864, 833 cm⁻¹.

Anal. Calcd for C₂₉H₃₃N₃O₃: C, 73.86; H, 7.05; N, 8.91.

Found: C, 73.73; H, 6.79; N, 8.76.

### Reference Example B14

2-(1-{4-[4-({2-[(E)-2-(4-fluorophenyl)ethenyl]-1,3-oxazol-4-yl}methoxy)phenyl]butyl)-1H-imidazol-2-yl)-1-ethanol

Using 4-[4-[2-(2-hydroxyethyl)-1H-imidazol-1-yl]butyl]phenol (391 mg), 65% oily sodium hydride (60 mg) and 4-(chloromethyl)-2-[(E)-2-(4-fluorophenyl)ethenyl]-1,3-oxazole (375 mg), the same reaction as Reference Example B2 was carried out to yield the titled compound (583 mg).

mp 130-132° C.

¹H-NMR(CDCl₃) δ: 1.56-1.84 (4H,m), 2.10-2.90 (1H,br}, 2.58 (2H,t,J=7.1 Hz), 2.78 (2H,t,J=5.5 Hz), 3.82 (2H,t,J=7.1 Hz), 4.03 (2H,t,J=5.5 Hz), 5.01 (2H,s), 6.80-6.94 (5H,m), 7.04-7.13 (4H,m), 7.46-7.55 (3H,m), 7.65 (1H,s).

IR (KBr): 3150, 3113, 3048, 2936, 2861, 1642, 1599, 1582, 1532, 1512, 1464, 1422, 1399, 1375, 1337, 1302, 1277, 1246, 1229, 1209, 1177, 1159, 1148, 1105, 1051, 1001 cm⁻¹.

Anal calcd for C₂₇H₂₈N₃O₃F: C,70.26;H,6.11;N,9.10.

Found: C,70.15;H,6.06;N,9.35

### Reference Example B15

2-[1-[4-[4-[[2-[(E)-2-(4-chlorophenyl)ethenyl]-1,3-oxazol-4-yl]methoxy]phenyl]butyl]-1H-imidazol-2-yl]-1-ethanol

To a solution of 4-[4-[2-(2-hydroxyethyl)-1H-imidazol-1-yl]butyl]phenol (130 mg) in DMF (4 mL), 60% oily sodium hydride (22 mg) was added under ice cooling. After stirring at room temperature for 30 minutes, (E)-4-chloromethyl-2-[2-(4-chlorophenyl)ethenyl]oxazole (140 mg) was added under ice cooling. After stirring at 0° C. for 1 hour, then at room temperature overnight, water was added under ice cooling. The precipitate was collected by filtration, washed with water, and dissolved in a mixture of THF-ethyl acetate. This solution was dried over magnesium sulfate, after which it was concentrated under reduced pressure. The residue was recrystallized from methanol-ethyl acetate-diethyl ether to yield the titled compound (168 mg) as colorless crystals.

mp 127-128° C.

¹H-NMR(CDCl₃) δ: 1.5-1.8 (4H, m), 2.58 (2H, t, J=7.0 Hz), 2.78 (2H, t, J=5.4 Hz), 3.82 (2H, t, J=7.0 Hz), 4.03 (2H, t, J=5.4 Hz), 5.01 (2H, s), 6.8-7.0-(5H, m), 7.07 (2H, d, J=8.8 Hz), 7.35 (2H, d, J=8.4 Hz), 7.46 (2H, d, J=8.4 Hz), 7.4-7.55 (1H, m), 7.66 (1H, s).

IR (KBr): 1514, 1474, 1341, 1264, 1246, 1076, 966, 814 cm⁻¹.

Anal. Calcd for C₂₇H₂₈ClN₃O₃: C, 67.85; H, 5;90; N, 8.79.

Found: C, 67.85; H, 5.72; N, 9.09.

### Reference Example B16

2-[1-[4-[4-[[2-[(E)-2-(4-bromophenyl)ethenyl]-1,3-oxazol-4-yl]methoxy]phenyl]butyl]-1H-imidazol-2-yl]-1-ethanol

To a solution of 2-(1H-imidazol-2-yl)-ethanol (449 mg) in DMF (10 mL), 60% oily sodium hydride (176 mg) was added under ice cooling. After stirring at room temperature for 30 minutes, 4-[[4-(4-iodobutyl)phenoxy]methyl]-2-[(E)-2-(4-bromophenyl)ethenyl]-1,3-oxazole (2.15 g) was added under ice cooling. After stirring overnight at room temperature, water was added under ice cooling. The reaction mixture was extracted with a mixture of ethyl acetate-THF. The extract was dried over magnesium sulfate, after which it was concentrated under reduced pressure. The residue was recrystallized from ethyl acetate-hexane to yield the titled compound (2.09 g) as light-yellow crystals.

mp 149-150° C.

¹H-NMR(CDCl₃) δ: 1.55-1.8 (4H, m), 2.58 (2H, t, J=7.0 Hz), 2.78 (2H, t, J=5.6 Hz), 3.82 (2H, t, J=7.0 Hz), 4.03 (2H, t, J=5.6 Hz), 5.01 (2H, s), 6.91 (2H, d, J=8.8 Hz), 6.92 (1H, d, J=16.3 Hz), 6.8-7.0 (2H, m), 7.07 (2H, d, J=8.8 Hz), 7.38 (2H, d, J=8.6 Hz), 7.47 (1H, d, J=16.3 Hz), 7.52 (2H, d, J=8.6 Hz), 7.66 (1H, s).

IR (KBr): 1514, 1487, 1254, 1055, 972, 826, 814 cm⁻¹.

Anal. Calcd for C₂₇H₂₈BrN₃O₃: C, 62.07; H, 5.40; N, 8.04.

Found: C, 61.82; H, 5.26; N, 7.90.

### Reference Example B17

2-[1-[4-[4-[2-[(E)-2-(4-trifluoromethylphenyl)ethenyl]oxazol-4-yl]methoxyphenyl]butyl-1H-imidazol-2-yl]-1-ethanol

In an argon atmosphere, DMF (4 mL) was added to a mixture of 65% sodium hydride (40.6 mg) and 4-[4-[2-(2-hydroxyethyl)-1H-imidazol-1-yl]butyl]phenol (260 mg) at 0° C. After stirring at room temperature for 30 minutes, [2-[(E)-2-(4-trifluoromethylphenyl)ethenyl]oxazol-4-yl]methyl chloride (316 mg) was added at 0° C., followed by stirring at room temperature for 15 hours. After water was added to the reaction mixture, the precipitated crystal was collected by filtration, washed with water and isopropyl ether, after which it was recrystallized from acetone-hexane to yield the titled compound (393 mg) as pale-yellow needles.

¹H-NMR (CDCl₃) δ: 1.56-1.74 (4H, m), 2.59 (2H, t, J=6.6 Hz), 2.78 (2H, t, J=5.4 Hz), 3.82 (2H, t, J=6.8 Hz), 4.03 (2H, t, J=5.4 Hz), 5.02 (2H, d, J=1.2 Hz), 6.81 (1H, d, J=1.6 Hz), 6.90-6.95 (4H, m), 7.02 (2H, d, J=16.2 Hz), 7.52-7.69 (6H, m).

IR (KBr): 1512, 1323, 1244, 1175, 1132, 1113, 1067, 1055 cm⁻¹.

### Reference Example B18

2-[1-[3-[4-[2-[(E)-2-(4-trifluoromethylphenyl)ethenyl]oxazol-4-yl]methoxyphenyl]propyl]-1H-imidazol-2-yl]-1-ethanol

Using 65% sodium hydride (40.6 mg), 4-[3-[2-(hydroxyethyl)-1H-imidazol-1-yl]propyl]phenol (246 mg) and [2-[(E)-2-(4-trifluoromethylphenyl)ethenyl]oxazol-4-yl]methyl chloride (316 mg), the same reaction as Reference Example B17 was carried out to yield the titled compound (330 mg) as colorless needles.

¹H-NMR (CDCl₃) δ: 2.01-2.08 (2H, m), 2.60 (2H, t, J=7.8 Hz), 2.74 (2H, t, J=5.8 Hz), 3.83 (2H, t, J=7.4 Hz), 4.03 (2H, t, J=5.8 Hz), 5.03 (2H, s), 6.84 (1H, d, J=1.2 Hz), 6.96-7.12 (6H, m), 7.52-7.70 (6H, m).

IR (KBr): 1512, 1327, 1246, 1173, 1125, 1069, 1017, 826 cm⁻¹.

### Reference Example B19

2-[1-[4-[4-[[2-[(E)-2-(2,4-difluorophenyl)ethenyl]-1,3-oxazol-4-yl]methoxy]phenyl]butyl]-1H-imidazol-2-yl]-1-ethanol

To a solution of 4-[4-[2-(2-hydroxyethyl)-1H-imidazol-1-yl]butyl]phenol (260 mg) in DMF (4 mL), 60% oily sodium hydride (44 mg) was added under ice cooling. After stirring at room temperature for 30 minutes, (E)-4-chloromethyl-2-[2-(2,4-difluorophenyl)ethenyl]oxazole (281 mg) was added under ice cooling. After stirring at room temperature for 3 days, water was added under ice cooling. The precipitate was collected by filtration and washed with water. The precipitate was dissolved in a mixture of ethyl acetate-THF and dried over magnesium sulfate, after which it was concentrated under reduced pressure. The residue was recrystallized from ethyl acetate-hexane to yield the titled compound (275 mg) as pale-yellow crystals.

mp 93-95° C.

¹H-NMR(CDCl₃) δ: 1.55-1.85 (4H, m), 2.58 (2H, t, J=7.0 Hz), 2.78 (2H, t, J=5.4 Hz), 3.82 (2H, t, J=7.0 Hz), 4.03 (2H, t, J=5.4 Hz), 5.01 (2H, s), 6.8-7.0 (6H, m), 6.98 (1H, d, J=16.3 Hz), 7.07 (2H, d, J=8.8 Hz), 7.5-7.6 (1H, m), 7.59 (1H, d, J=16.3 Hz), 7.67 (1H, s).

IR (KBr): 1611, 1508, 1277, 1231, 1140, 1103, 1063, 970, 860 cm⁻¹.

Anal. Calcd for C₂₇H₂₇F₂N₃O₃.0.1H₂O: C, 67.38; H, 5.70; N, 8.73.

Found: C, 67.24; H, 5.74; N, 8.55.

### Reference Example B20

2-[1-[3-[4-[[2-[(E)-2-(2,4-difluorophenyl)ethenyl]-1,3-oxazol-4-yl]methoxy]phenyl]propyl]-1H-imidazol-2-yl]-1-ethanol

To a solution of 4-[4-[2-(2-hydroxyethyl)-1H-imidazol-1-yl]propyl]phenol (246 mg) in DMF. (4-mL), 60% oily sodium hydride (44 mg) was added under ice cooling. After stirring at room temperature for 30 minutes, (E)-4-chloromethyl-2-[2-(2,4-difluorophenyl)ethenyl]oxazole (281 mg) was added under ice cooling. After stirring overnight at room temperature, water was added under ice cooling. The precipitate was collected by filtration and washed with water. The precipitate was dissolved in ethyl acetate and dried over magnesium sulfate, after which it was concentrated under reduced pressure. The residue was recrystallized from ethyl acetate-diethyl ether-hexane to yield the titled compound (272 mg) as colorless crystals.

mp 94-96° C.

¹H-NMR(CDCl₃) δ: 1.95-2.15 (2H, m), 2.5-2.65 (2H, m), 2.65-2.8 (2H, m), 3.75-3.9 (2H,m), 3.95-4.1 (2H, m), 5.02 (2H, s), 6.8-7.15 (9H, m), 7.45-7.7 (3H., m).

IR(KBr): 1609, 1512, 1277, 1231, 1140, 1061, 1020, 974, 860 cm⁻¹.

Anal. Calcd for C₂₆H₂₅F₂N₃O₃.0.4H₂O: C, 66.06; H, 5.50; N, 8.89.

Found: C, 66.13; H, 5.38; N, 8.55.

### Reference Example B21

2-[1-[3-[4-[[2-[(E)-2-(2,6-difluorophenyl)ethenyl]-1,3-oxazol-4-yl]methoxy]phenyl]propyl]-1H-imidazol-2-yl]-1-ethanol

Using 2-(2-hydroxyethyl)-1-[4-(4-hydroxyphenyl)butyl]imidazole (260 mg), 60% oily sodium hydride (41 mg) and (E)-4-chloromethyl-2-[2-(2,6-difluorophenyl)ethenyl]oxazole (281 mg), the same reaction as Reference Example B19 was carried out to yield the titled compound (359 mg) as colorless crystals.

mp 106-107° C.

¹H-NMR(CDCl₃) δ: 1.5-1.8 (4H, m), 2.58 (2H, t, J=7.0 Hz), 2.78 (2H, t, J=5.6 Hz), 3.82 (2H, t, J=7.0 Hz), 4.03 (2H, t, J=5.6 Hz), 5.02 (2H, s), 6.8-7.0 (6H, m), 7.07 (2H, d, J=8.4 Hz), 7.2-7.35 (2H, m), 7.61 (1H, d, J=16.8 Hz), 7.68 (1H, s).

IR (KBr): 1618, 1516, 1472, 1456, 1246, 1065, 1001, 974, 789 cm⁻¹.

Anal calcd for C₂₇H₂₇F₂N₃O₃: C, 67.63; H, 5.68; N, 8.76.

Found: C, 67.78; H, 5.57; N, 9.01.

### Reference Example B22

3-(4-{4-[4-({2-[(E)-2-(3-methylphenyl)ethenyl]-1,3-oxazol-4-yl}methoxy)phenyl]butyl}-1H-imidazol-2-yl)-1,2-propanediol

Using 3-(1-[4-(4-hydroxyphenyl)butyl]-1H-imidazol-2-yl]-1,2-propanediol (154 mg), 65% oily sodium hydride (21 mg) and 4-(chloromethyl)-2-[(E)-2-(3-methylphenyl)ethenyl]-1,3-oxazole (131 mg), the same reaction as Reference Example B2 was carried out to yield the titled compound (156 mg).

mp 102-104° C.

¹H-NMR(CDCl₃) δ: 1.52-1.82 (4H,m), 2.39 (3H,s), 2.59 (2H,t,J=7.0 Hz), 2.77 (1H,d,J=5.0 Hz), 2.78 (1H,d,J=6.8 Hz), 3.64 (1H,dd,J=4.8 Hz, 11.2 Hz), 3.76 (1H,dd,J=4.2 Hz, 11.2 Hz), 3.82 (2H,t,J=7.0 Hz), 4.12-4.24 (1H,m), 5.02 (2H,s), 6.80 (1H,d,J=1.4 Hz), 6.92 (1H,d,J=1.4 Hz), 6.93 (1H,d,J=16.2 Hz), 6.93 (1H,d,J=8.8 Hz), 7.08 (2H,d,J=8.8 Hz), 7.13-7.39 (4H,m), 7.52 (1H,d,J=16.2 Hz), 7.66 (1H,s).

IR (KBr): 3500-3200, 3112, 3029, 2934, 2865, 1645, 1609, 1584, 1510, 1491, 1462, 1379, 1350, 1242, 1177, 1150, 1123, 1100, 1026 cm⁻¹.

Anal calcd for C₂₉H₃₃N₃O₄.0.5H₂O: C,70.14;H,6.90;N,8.46.

Found: C,70.39;H,6.63;N,8.51.

### Reference Example B23

3-(1-{4-[4-({2-[(E)-2-(4-fluorophenyl)ethenyl]-1,3-oxazol-4-yl}methoxy)phenyl]butyl}-1H-imidazol-2-yl)-1,2-propanediol

Using 3-{1-[4-(4-hydroxyphenyl)butyl]-1H-imidazol-2-yl}-1,2-propanediol (291 mg), 65% oily sodium hydride (39 mg) and 4-(chloromethyl)-2-[(E)-2-(4-fluorophenyl)ethenyl]-1,3-oxazole (250 mg), the same reaction as Reference Example B2 was carried out to yield the titled compound (347 mg).

mp 114-116° C.

¹H-NMR(CDCl₃) δ: 1.52-1.83 (4H,m), 2.59 (2H,t,J=7.2 Hz), 2.76 (1H,d,J=5.2 Hz), 2.77 (1H,d,J=7.0 Hz), 3.64 (1H,dd,J=4.8 Hz, 11.4 Hz), 3.76 (1H,dd,J=4.2 Hz, 11.4 Hz), 3.82 (2H,t,J=6.8 Hz), 4.12-4.24 (1H,m), 5.01 (2H,s), 6.80 (1H,d,J=1.4 Hz), 6.86 (1H,d,J=16.8 Hz), 6.92 (1H,d,J=1.4 Hz), 6.93 (2H,d,J=8.8 Hz), 7.07 (2H,d,J=8.8 Hz), 7.09 (2H,d,J=8.7 Hz), 7.46-7.56 (3H,m), 7.66(1H,s).

IR (KBr): 3500-3200, 3152, 3104, 3044, 2940, 2865, 1644, 1599, 1584, 1532, 1512, 1495, 1462, 1422, 1400, 1339, 1300, 1246, 1177, 1159, 1098, 1047 cm⁻¹.

Anal calcd for C₂₈H₃₀N₃O₄F: C,68.42;H,6.15;N,8.55.

Found: C,68.16;H,5.98;N,8.46.

### Reference Example B24

3-[1-(4-(4-[(2-{(E)-2-[4-(trifluoromethyl)phenyl]ethenyl}-1,3-oxazol-4-yl)methoxy]phenyl]butyl)-1H-imidazol-2-yl]-1,2-propanediol

Using 3-{1-[4-(4-hydroxyphenyl)butyl]-1H-imidazol-2-yl}-1,2-propanediol (204 mg), 65% oily sodium hydride (28 mg) and 4-(chloromethyl)-2-{(E)-2-[4-(trifluoromethyl)phenyl]ethenyl}-1,3-oxazole (212 mg), the same reaction as Reference Example B2 was carried out to yield the titled compound (285 mg).

mp 142-143° C.

¹H-NMR(CDCl₃) δ: 1.53-1.82 (4H,m), 2.59 (2H,t,J=7.1 Hz), 2.76 (1H,d,J=5.0 Hz), 2.77 (1H,d,J=7.0 Hz), 3.64 (1H,dd,J=4.8 Hz, 11.4 Hz), 3.76 (1H,dd,J=4.2 Hz, 11.4 Hz), 3.83 (2H,t,J=6.8 Hz), 4.12-4.24 (1H,m), 5.02 (2H,s), 6.81 (1H,d,J=1.4 Hz), 6.92 (1H,d,J=1.4 Hz), 6.93 (2H,d,J=8.8 Hz), 6.95 (1H,d,J=16.4 Hz),7.08 (2H,d,J=8.8 Hz), 7.56 (1H,d,J=16.4 Hz), 7.64 (4H,s), 7.70 (1H,s).

IR (KBr): 3500-3200, 3148, 3071, 2936, 2867, 1642, 1615, 1582, 1510, 1491, 1466, 1416, 1397, 1323, 1246, 1173, 1138, 1117, 1067, 1046, 1017 cm⁻¹.

Anal calcd for C₂₉H₃₀N₃O₄F₃: C,64.32;H,5.58;N,7.76.

Found: C,64.26;H,5.70;N,7.62.

### Reference Example B25

3-[1-(3-{3-[(2-{(E)-2-[4-(trifluoromethyl)phenyl]ethenyl}-1,3-oxazol-4-yl)methoxy]phenyl}propyl)-1H-imidazol-2-yl]-1,2-propanediol

Using 3-{1-[3-(3-hydroxyphenyl)propyl]-1H-imidazol-2-yl}-1,2-propanediol (194 mg), 65% oily sodium hydride (28 mg) and 4-(chloromethyl)-2-{(E)-2-[4-(trifluoromethyl)phenyl]ethenyl)-1,3-oxazole (212 mg), the same reaction as Reference Example B2 was carried out to yield the titled compound (255 mg).

mp 102-104° C.

¹H-NMR(CDCl₃) δ: 2.08 (2H,quintet, J=7.0 Hz), 2.62 (2H,t,J=7.4 Hz), 2.72 (1H,d,J=4.8 Hz), 2.73 (1H,d,J=7.6 Hz), 3.63 (1H,dd,J=4.8 Hz, 11.4 Hz), 3.74 (1H,dd,J=4.2 Hz, 11.4 Hz), 3.83 (2H,t,J=7.2 Hz), 4.13-4.24 (1H,m), 5.03 (2H,s), 6.77-6.91 (3H,m), 6.84 (1H,d,J=1.4 Hz), 6.94 (1H,d,J=1.4 Hz), 7.02 (1H,d,J=16.4 Hz), 7.25 (1H,t,J=7.8 Hz), 7.57 (1H,d,J=16.4 Hz), 7.64 (4H,s), 7.71 (1H,s)

IR (KBr): 3500-3200, 3108, 3056, 2932, 2867, 1613, 1599, 1586, 1534, 1489, 1451, 1416, 1325, 1260, 1167, 1125, 1069, 1030, 1017 cm⁻¹.

Anal calcd for C₂₈H₂₈N₃O₄F₃: C,63.75;H,5.35;N,7.97.

Found: C,63.60;H,5.32;N,7.88.

### Reference Example B26

3-(1-{4-[4-({2-[(E)-2-(2,4-difluorophenyl)ethenyl]-1,3-oxazol-4-yl}methoxy)phenyl]butyl}-1H-imidazol-2-yl)-1,2-propanediol

Using 3-{1-[4-(4-hydroxyphenyl)butyl]-1H-imidazol-2-yl}-1,2-propanediol (204 mg), 65% oily sodium hydride (28 mg) and 4-(chloromethyl)-2-[(E)-2-(2,4-difluorophenyl)ethenyl]-1,3-oxazole (188 mg), the same reaction as Reference Example B2 was carried out to yield the titled compound (223 mg).

mp 126-128° C.

¹H-NMR(CDCl₃) δ: 1.52-1.81 (4H,m), 2.58 (2H,t,J=6.9 Hz), 2.77 (2H,d,J=5.4 Hz), 3.63 (1H,dd,J=4.8 Hz, 11.4 Hz), 3.75 (1H,dd,J=4.2 Hz, 11.4 Hz), 3.82 (2H,t,J=7.0 Hz), 4.10-4.24 (1H,m), 5.01 (2H,s), 6.76-7.02 (7H,m), 7.07 (2H,d,J=8.6 Hz), 7.48-7.51 (1H,m), 7.59 (1H,d,J=16.6 Hz), 7.67 (1H,s).

IR (KBr): 3500-3200, 3106, 3073, 3032, 2934, 2865, 1644, 1613, 1593, 1532, 1512, 1495, 1462, 1431, 1354, 1298, 1275, 1244, 1177, 1142, 1090, 1028 cm⁻¹.

Anal calcd for C₂₈H₂₉N₃O₄F₂: C,66.00;H,5.74;N,8.25.

Found: C,65.89;H,5.94;N,8.37.

### Reference Example B27

3-(1-{3-[3-({2-[(E)-2-(2,4-difluorophenyl)ethenyl]-1,3-oxazol-4-yl}methoxy)phenyl]propyl}-1H-imidazol-2-yl)-1,2-propanediol

Using 3-{1-[3-(3-hydroxyphenyl)propyl]-1H-imidazol-2-yl}-1,2-propanediol (203 mg), 65% oily sodium hydride (29 mg) and 4-(chloromethyl)-2-[(E)-2-(2,4-difluorophenyl)ethenyl]-1,3-oxazole (197 mg), the same reaction as Reference Example B2 was carried out to yield the titled compound (220 mg).

mp 92-94° C.

¹H-NMR(CDCl₃) δ: 2.08 (2H,quintet,J=7.2 Hz), 2.62 (2H,t,J=7.3 Hz), 2.73 (1H,d,J=5.0 Hz), 2.74 (1H,d,J=7.0 Hz), 3.63 (1H,dd,J=4.8 Hz, 11.2 Hz), 3.74 (1H,dd,J=4.2 Hz, 11.2 Hz), 3.83 (2H,t,J=7.4 Hz), 4.14-4.24 (1H,m), 5.02 (2H,s), 6.76-6.98 (5H,m), 6.84 (1H,d,J=1.4 Hz), 6.93 (1H,d,J=1.4 Hz), 6.98 (1H,d,J=16.4 Hz), 7.25 (1H,t,J=7.9 Hz), 7.48-7.61 (1H,m), 7.60 (1H,d,J=16.4 Hz), 7.69 (1H,s).

IR (KBr): 3500-3200, 3106, 3067, 3042, 2938, 2872, 1644, 1613, 1599, 1534, 1495, 1453, 1431, 1379, 1354, 1275, 1155, 1142, 1123, 1090, 1028 cm⁻¹.

Anal calcd for C₂₇H₂₇N₃O₄F₂: C,65.44;H,5.49;N,8.48.

Found: C,65.39;H,5.32;N,8.62.

### Reference Example B28

3-[1-[4-[4-[[2-[(E)-2-(2,6-difluorophenyl)ethenyl]-1,3-oxazol-4-yl]methoxy]phenyl]butyl]-1H-imidazol-2-yl]-1,2-propanediol

Using 3-(1-[3-(3-hydroxyphenyl)propyl]-1H-imidazol-2-yl}-1,2-propanediol (142 mg), 60% oily sodium hydride (40 mg) and 4-(chloromethyl)-2-[(E)-2-(2,6-difluorophenyl)ethenyl]-1,3-oxazole (495 mg), the same reaction as Reference Example B2 was carried out to yield the titled compound (395 mg) as colorless crystals.

mp 123-125° C.

¹H-NMR(CDCl₃) δ: 1.5-1.8 (4H, m), 2.59 (2H, t, J=7.0), 2.7-2.8 (2H, m), 3.6-3.75 (2H, m), 3.83 (2H, t, J=7.0 Hz), 4.1-4.25 (1H, m), 5.03 (2H, s), 6.8-7.0 (4H, m), 6.92 (2H, d, J=8.6 Hz), 7.07 (2H, d, J=8.6 Hz), 7.2-7.3 (1H, m), 7.29 (1H, d, J=16.8 Hz), 7.61 (1H, d, J=16.8 Hz), 7.69 (1H, s).

IR (KBr): 1620, 1508, 1458, 1236, 1051, 1001, 789 cm⁻¹.

Anal. Calcd for C₂₈H₂₉F₂N₃O₄: C, 6.6.00; H 5.74; N, 8.25.

Found: C, 65.71; H, 5.78; N, 8.09.

### Reference Example B29

(2R)-3-[[1-[4-[4-[[2-[(E)-2-(2,4-difluorophenyl)ethenyl]-1,3-oxazol-4-yl]methoxy]phenyl]butyl]-1H-imidazol-2-yl]-1,2-propanediol

To a solution of (2R)-3-(1H-imidazol-2-yl)-1,2-propanediol (127 mg) in DMF (4 mL), 60% oily sodium hydride (37 mg) was added under ice cooling. After stirring at room temperature for 30 minutes, 4-[[4-(4-iodobutyl)phenoxy]methyl]-2-[(E)-2-(2,4-difluorophenyl)ethenyl]-1,3-oxazole (485 mg) was added under ice cooling. After stirring at room temperature for 3 hours, water was added under ice cooling. The reaction mixture was extracted with a mixture of THF-ethyl acetate and washed with water and saline and dried over magnesium sulfate, after which it was concentrated under reduced pressure. The residue was purified by silica gel column chromatography (eluent: ethyl acetate-methanol=10:1), after which it was recrystallized from ethyl acetate-hexane to yield the titled compound (262 mg) as colorless crystals.

mp 104-106° C.

¹H-NMR(CDCl₃) δ: 1.5-1.8 (4H, m), 2.59 (2H, t, J=7.0 Hz), 2.7-2.8 (2H, m), 3.55-3.75 (2H, m), 3.79 (2H, t, J=7.0 Hz), 4.1-4.2 (1H, m), 5.01 (2H, s), 6.8-7.1 (5H, m), 6.92 (2H, d, J=8.4 Hz), 7.07 (2H, d, J=8.4 Hz), 7.5-7.6 (1H, m), 7.59 (1H, d, J=16.2 Hz), 7.67 (1H, s).

IR (KBr): 1507, 1472, 1273, 1235, 1140, 1092, 966, 858 cm⁻¹.

Anal. Calcd for C₂₈H₂₉F₂N₃O₄: C, 66.00; H, 5.74; N, 8.25.

Found: C, 65.69; H, 5.82; N, 8.06.

[α]_{D}²²=+4.20 (c=1.0, methanol)

### Reference Example B30

(2S)-3-[[1-[4-[4-[[2-[(E)-2-(2,4-difluorophenyl)ethenyl]-1,3-oxazol-4-yl]methoxy]phenyl]butyl]-1H-imidazol-2-yl]-1,2-propanediol

Using (2S)-3-(1H-imidazol-2-yl)-1,2-propanediol, 60% oily sodium hydride (50 mg) and 4-[[4-(4-iodobutyl)phenoxy]methyl]-2-[(E)-2-(2,4-difluorophenyl)ethenyl]-1,3-oxazole (415 mg), the same reaction as Reference Example B29 was carried out to yield the titled compound (219 mg) as colorless crystals.

mp 106-108° C.

¹H-NMR(CDCl₃) δ: 1.5-1.8 (4H, m), 2.58 (2H, t, J=6.8 Hz), 2.7-2.8 (2H, m), 3.6-3.75 (2H, m), 3.82 (2H, t, J=7.0 Hz), 4.1-4.2 (1H, m), 5.01 (2H, s), 6.8-7.1 (5H, m), 6.89 (2H, d, J=8.4 Hz), 7.07 (2H, d, J=8.4 Hz), 7.5-7.6 (1H, m), 7.59 (1H, d, J=16.4 Hz), 7.67 (1H, s).

IR(KBr): 1615, 1512, 1497, 1273, 1246, 1229, 1140, 1094, 1046, 966, 847 cm⁻¹.

Anal. Calcd for C₂₈H₂₉F₂N₃O₄: C, 66.00; H, 5.74; N, 8.25. Found: C, 65.75; H, 5.60; N, 8.12.

[α]_{D}²²=−3.5° (c=1.0, methanol).

### Reference Example C1

### Suppression of Receptor Tyrosine-Phosphorylation in Human Breast Cancer Cells

Cell suspension of Human breast cancer cell line MCF-7 (500 μl), suspended at 300,000 cells/0.5 mL, was placed in a 24-well plate and cultured at 37° C. in a 5% carbon dioxide gas incubator. On the following day, 250 μl of a solution of 4-fold serially diluted test compound was added. After 2 hours, 250 μl of a heregulin solution, prepared to a final concentration of 0.8 μg/ml, was added. After 5 minutes, a buffer solution for lysis of extract solution was added to stop the reaction and to extract and yield a cell-lysate protein. After this cell-lysate protein was subjected to SDS-polyacrylamide gel electrophoresis to separate the protein, the protein in the gel was blotted onto a nylon filter. This filter was reacted with an anti-phosphotyrosine specific antibody; the portion containing phosphotyrosine on the filter was luminated using the ECL method to photosensitize an X-ray film. The amount of the film photosensitization was determined using an image analyzer. Taking as 100% the amount of phosphorylation of the HER2 tyrosine in the heregulin added group, the ratio of the amount of phosphorylation of the HER2 tyrosine in each group receiving a solution of the test compound at each concentration was determined, and the test compound concentration required to achieve 50% suppression of the amount of phosphorylation of HER2 tyrosine (IC₅₀ value) was calculated.

The results are shown in Table 1.

This finding showed that the compound (I) potently inhibits the phosphorylation reaction of the tyrosine residue of the receptor protein caused by activation of the receptor tyrosine kinase due to growth factor stimulation upon stimulation of human breast cancer cells by the growth factor heregulin.

TABLE 1

| | Intracellular HER2 phosphorylation |
|---|---|
| Ref. Ex. No. | inhibition |
| (Compound No.) | MCF-7 (IC₅₀:μM) |
| B2 | 1.9 |
| B3 | 0.18 |
| B4 | 0.10 |
| B6 | 1.2 |
| B11 | 1.1 |
| B20 | 1.5 |
| B22 | 1.9 |
| B26 | 0.92 |

### Reference Example C2

### Inhibitory Action on Breast Cancer Cell Line BT-474 Proliferation In Vitro (In Vitro)

Cell suspension of human breast cancer cell line BT-474 (100 μl) suspended at 1,000 cells/100 μl), was placed in a 96-well microwell plate and cultured at 37° C. in a 5% carbon dioxide gas incubator. On the following day, 100 μl of a solution of each test compound, previously diluted 2-fold with a heregulin solution prepared to a final concentration of 0.04 μg/ml, was added, and the cells were cultured for 5 days. After the culture medium containing the test compound was removed, the cells were washed and fixed with 5% trichloroacetic acid, after which a 0.4% (w/v) SRB solution (dissolved in 1% acetic acid) was added to fix and stain the cells (Skehan et al., Journal of the National Cancer Institute, Vol. 82, pp. 1107-1112, 1990). After the pigment solution was removed and the plate was washed with a 1% acetic acid solution, 100 μl of an extractant (10 mM Tris buffer solution) was added to extract and dissolve the pigment; absorbance was measured at an absorption wavelength of 550 nm to quantify the amount of cells as protein content. Taking as 100% the absorbance for the control group, which received no test compound solution, the ratio of the absorbance for each treatment group was determined, and the compound concentration required to achieve 50% suppression of the residual cell content relative to the control (IC₅₀ value) was calculated.

The results are shown in Table 2.

The compound (I) was thus shown to potently suppress the proliferation of the human breast cancer cell line BT-474.

TABLE 2

| Ref. Ex. No. | Inhibition of cell proliferation |
|---|---|
| (Compound No.) | BT-474 (IC₅₀:μM) |
| B2 | <0.05 |
| B3 | <0.05 |
| B4 | <0.05 |
| B6 | <0.05 |
| B11 | <0.05 |
| B19 | 0.017 |
| B20 | <0.05 |
| B22 | <0.05 |
| B26 | <0.05 |

### Reference Example C3

### Inhibitory Action on Breast Cancer Cell Proliferation In Vivo (In Vivo)

5,000,000 cells of human breast cancer cell line BT-474 were suspended in Matrigel solution and transplanted subcutaneously into the chest of a female BALB/c nude mouse (6 weeks of age) (Freedman et al., Proceedings of the National Academy of Science, USA, Vol. 87, pp. 6698-6702, 1990).

Immediately after transplantation and at 7 days after transplantation, 50 μL of estradiol dipropionate (5 mg/mL solution) was administered intramuscularly into a hind leg in an attempt to enhance the survival of the tumor. At 14 days after transplantation, tumor diameter was measured, and 5 mice per group, uniformized with respect to tumor size, were used for the experiment. The compound of the present invention in a 5% gum arabic suspension (physiological saline) was administered orally at a dose of 30 mg/kg twice daily for 10 days. On the day of administration initiation and the day after administration completion, tumor diameter was measured, and tumor volume was calculated using the equation shown below.

Formula: Tumor volume=longer diameter×shorter diameter×shorter diameter×(1/2)

The ratio of the value obtained by subtracting the tumor volume on the day of administration initiation from the tumor volume on the day after administration completion in the control group, which received an gum arabic solution, and to that the value obtained by subtracting the tumor volume at the day of administration initiation from the tumor volume at the day of administration completion in each drug administration group was obtained as the proliferation rate.

The results are shown in Table 3.

The compound (I) suppressed the growth of human breast cancer cells transplanted to nude mice. Mice were weighed during the test period; no body weight loss due to administration of the compound (I) was observed.

TABLE 3

| Ref. Ex. No. (Compound. No.) | Growth rate (%) |
|---|---|
| B4 | 5 |
| B6 | 28 |
| B23 | 27 |
| B24 | 28 |
| B26 | 15 |

### Reference Example C4

### Enzyme Selectivity of Tyrosine Kinase Inhibitory Activity of Compound B4

For consideration of the action on a receptor-type tyrosine kinase, A-431 cells or NIH3T3 cells were sown in a 24 multi-well plate (2×10⁵ cells/well) and cultured overnight in a carbon dioxide gas incubator (5% CO₂, 37° C.). The medium was changed to a serum-free medium and the cells were further incubated for one day. Compound B4 (compound of Ref. Ex. No. (Compound No.) B4; hereinafter the same) was added to a given concentration, and after 2 hr, A-431 cells were stimulated with EGF (20 ng/mL) and NIH3T3 cells were stimulated with PDGF (20 ng/mL) or FGF (20 ng/mL). The culture medium was removed 5 min later and SDS sample buffer was added to give a cell lysate (200 μL). For consideration of the action on a non-receptor type tyrosine kinase, A431 (Jak1), Jurkat (Src) or Namalwa cells (Blk) were sown in a 6-well multi-well plate (5×10⁵ cells/well) and cultured overnight in a carbon dioxide gas incubator (5% CO₂, 37° C.). Compound B4 was added to a given concentration, and after 2 hr, the culture medium was removed and SDS sample buffer was added to give a cell lysate (200 μL). The cell lysate (8 μL) was separated by SDS-PAGE (7.5%-15%) and, after western blotting, each phosphorylated tyrosine kinase was detected by the ECL™ system (Amersham) and the amount of phosphorylation was quantified by image analysis.

The amount of phosphorylation at each point was converted into a percentage with the amount of phosphorylation without the compound B4 treatment as 100%, and the concentration of compound B4, which affords 50% inhibition was calculated and taken as an IC₅₀ value. The results are shown in the following Table 4.

The IC₅₀ value of compound B4 for tyrosine kinase activity other than HER2 was higher than 25 μmol/L in every case.

The tyrosine kinase inhibitory activity of compound B4 was HER2 selective.

TABLE 4

| Tyrosine kinase | IC₅₀ (μmol/L) |
|---|---|
| EGFR | >25 |
| FGFR | >25 |
| PDGFR | >25 |
| Jak1 | >25 |
| Src | >25 |
| blk | >25 |

### Reference Example C5

### Her2 Expression Cell Line Selectively or HER2 Non-Expression Cell Line Selectivity (Intracellular Signal Transduction) of Compound B4

HER2 expression human cancer cell line (BT-474) was sown in a 24-well multi-well plate (BT-474:1.2×10⁵ cells/well) and cultured overnight in a carbon dioxide gas incubator (5% CO₂, 37° C.). Compound B4 was added, and after 2 hr, the culture medium was removed and the cells were dissolved in SDS sample buffer (200 μL). The cell lysate (8 μL) was separated by SDS-PAGE (7.5-15%) and, after western blotting, HER2 phosphorylation and phosphorylated Akt were detected by the 25 ECL™ system and quantified by image analysis. The results are shown in FIG. 1.

The phosphorylation of HER2 was remarkable in excessively expressed cell line (BT-474 cells), showing suppression by compound B4 (IC₅₀ value: 4.0 nmol/L). The compound B4 inhibited activation of Akt in HER2 expression cells (IC₅₀ value: 1.9 nmol/L).

Compound B4 suppressed activation of anti-apoptosis factor Akt located downstream of HER2 in the signal transduction system of cancer cells.

### Reference Example C6

### Cell Death Induction Activity by Intracellular Signal Transduction Inhibition of Compound B4

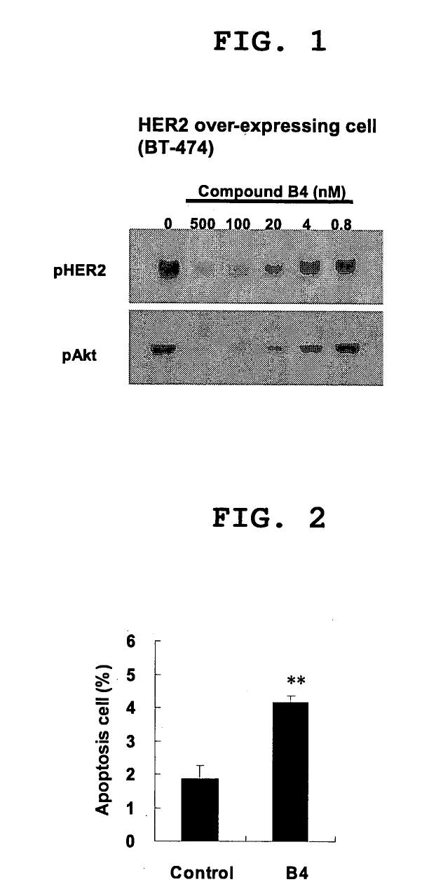

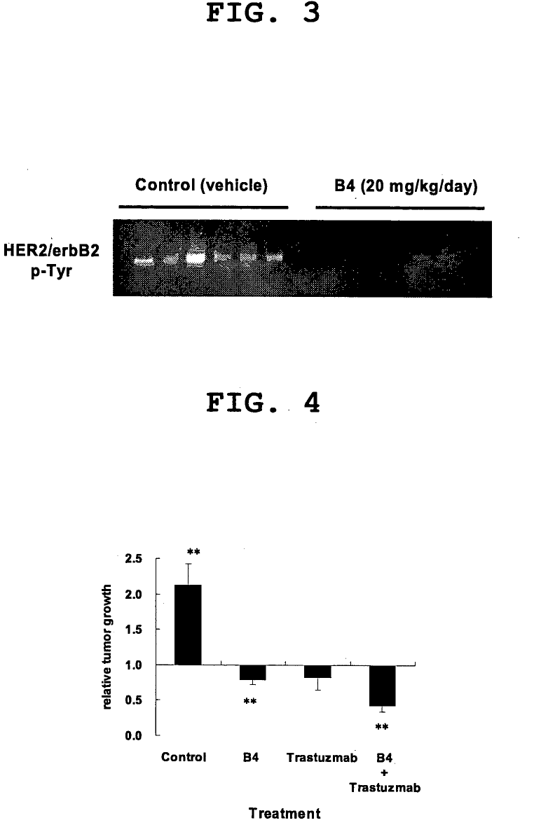

HER2 expression human cancer cell line is sown in a 10 cm culture dish (BT-474: 1×10⁶ cells/dish) and cultured overnight in a carbon dioxide gas incubator (5% CO₂, 37° C.). Compound B4 (1 μmol/L) was added and the cells were further cultured for ˜5 days. After the culture, the culture medium was removed and a cell suspension was prepared by a trypsin treatment and the cells were fixed with 70% ethanol. The DNA of the cells was stained with propidium iodide and the DNA amount was measured by a flow cytometer. The results are shown in FIG. 2. The cells with a decreased amount of DNA are apoptotic (cell death) cells.

The compound B4 induced cell death of HER2 high expression human breast cancer cells BT-474. This is considered to be attributable to the inhibition of HER2 tyrosine kinase by compound B4, and the inhibition of intracellular signal transduction (Akt activated) accompanying therewith. The compound B4 inhibits HER2 in the signal transduction system of cancer cells, and suppresses activation of anti-apoptosis factor Akt located downstream of HER2. As a result, cancer cells develop G1 arrest and the cell death is induced.

The compound B4 is a useful compound having an activity of inducing cell death to cancer cells through HER2 inhibition.

### Reference Example C7

### Intra-Tumor Inhibition of HER2 Phosphorylation by Compound B4

The compound B4 (20 mg/kg/day, divided-into two per day) was orally administered for 14 consecutive days to breast cancer cell line BT-474 gallbladder cancer nude mice (6 per group) and the tumor volume before administration and the tumor volume on the next day of the final administration were measured to confirm an anti-tumor effect. Thereafter, compound B4 (10 mg/kg, one dose of two divisions) was given. Four hours later, the tumor was sampled and a tumor homogenate was prepared. The tumor homogenate was separated by SDS-PAGE (7.5-15%), and after western blotting, HER2 and phosphorylated HER2 were detected by the ECL™ system and quantified by image analysis. The results are shown in FIG. 3.

The intra-tumor HER2 phosphorylation (per tumor weight) then of the compound B4 administration group was inhibited to 13% of the control group (87% inhibition).

It is clear that the target molecule HER2 can be inhibited also in tumor by administration of compound B4.

### Reference Example C8

### Anti-Tumor Effect of Compound B4 (Combined Use)

The compound B4 was orally administered (20 mg/kg/day, divided into two per day) for 14 days, or trastuzumab (Herceptin (product name)) (10 mg/kg, twice a week) was intraperitoneally administered for 2 weeks (4 times in total), or both compound B4 and trastuzumab were simultaneously administered to HER2 high expression human breast cancer cell BT-474 cancer-bearing nude mice. An increase in the tumor volume from that before administration to that on the next day of the final administration was compared to that of the control group and anti-tumor effect was studied. In addition, the tumor volume was calculated from the following equation:

Formula: Tumor volume=longer diameter×shorter diameter×shorter diameter×(1/2)

The ratio of the value obtained by subtracting the tumor volume on the day of administration initiation from the tumor volume on the next day of the completion of the administration in the control group, and the value obtained by subtracting the tumor volume on the day of administration initiation from the tumor volume on the next day of the completion of the administration was determined as a growth rate (T/C (%)). The results are shown in FIG. 4.

A ratio (T/C (%)) of the administration group relative to the control group with regard to the increase in the tumor volume from the tumor volume before administration to that on the next day of the final administration was calculated. As a result, compound B4 single group (T/C=−18%), trastuzumab single group (T/C=−17%) and the combination group (T/C=−46%) each significantly suppressed the tumor growth. In the compound B4 single group and combination group, a significant decrease in the tumor volume (5 cases out of 5 cases) (compound B4 single group: 13-30% reduction, combination group: 51-67% reduction). The action mechanism of compound B4 is different from that of trastuzumab. The combined use of compound B4 and an anti-cancer drug having a different action mechanism resulted in a stronger anti-tumor activity as compared to a single administration.

As shown above, the combined use of compound B4 and trastuzumab is highly useful as an anti-tumor agent.

### Reference Example C9

### Cell Growth Inhibitory Action of Compound B4 on HER2 Expression Cells (Cell Panel)

1) Cell growth inhibitory action; Each cell line was sown in a 24-well multi-well plate (5×10⁴ to 2×10⁵ cells/well) and cultured overnight in a carbon dioxide gas incubator (5% CO₂, 37° C.). The compound B4 was added and the cells were further cultured overnight in a carbon dioxide gas incubator for 3-5 days, after which the cells were counted.

2) Scoring HER2 expression; cell growth each cell line was sown in a multi-chamber slide (5×10³ cells/chamber), and after culturing overnight in a carbon dioxide gas incubator (5% CO₂, 37° C.), fixed with formalin. According to the manual attached to HercepTest™, the cells were stained and the level of HER2 expression was scored under a microscope. The results are shown in Table 5. Score 0 means HER2 non-expression, 1+ or above means HER2 positive and 3+means the highest expression amount.

The compound B4 showed cell growth inhibitory activity in every cancer studied (breast cancer, lung cancer, pancreatic cancer, kidney cancer, ovarian cancer, colon cancer). It also showed cell growth inhibitory activity in childhood cancers such as Wilms tumor, teratoid tumor and the like. The compound B4 showed cell growth inhibitory activity in HER2 positive cell lines having a HercepTest™ score of 1+ of above and this activity was not limited to specific cancers. However, it did not show effect on the growth of a HER2 non-expression cell.

The compound B4 is effective against HER2 positive tumors from among many malignant solid tumors including breast cancer, which are associated with bad prognosis.

TABLE 5

| | | HER2 | Compound B4 |
|---|---|---|---|
| Cancer | Cell line | score | IC₅₀ (μmol/L) |
| Breast | BT-474 | 3+ | 0.05 |
| | MDA-MB-231 | 0 | >25 |
| Lung | A549 | 1+ | 0.008 |
| | Ma-1 | 0 | >25 |
| Pancreas | MIA-PaCa-2 | 1+ | 0.02 |
| | BxPC-3 | 0 | >25 |
| Renal | ACHN | 1+ | 0.14 |
| | 786-O | 0 | >25 |
| Ovarian | NIH:OV-CAR-3 | 1+ | 0.06 |
| | ES-2 | 0 | 12 |
| Colon | HCT-116 | 1+ | <0.05 |
| | SW480 | 0 | >25 |
| Wilms' | G401 | 1+ | <0.05 |
| Teratoma | PA-1 | 1+ | <0.05 |

### Reference Example C10

### Anti-Tumor Effect of Compound B4 Against HER 2 Expression Tumor (Lung Cancer-Breast Cancer Gastric Cancer)

HER2 high expression human breast cancer cells BT-474 (10,000,000 cells) were suspended in Matrigel solution and subcutaneously transplanted into the chest of female BALB/c nude mouse (6 weeks of age). Immediately after transplantation and day 7 after the transplantation, estradiol dipropionate (5 mg/kg solution, 50 μL) was intramuscularly administered to the hind leg with the aim of enhancing the survival rate of the tumor. HER2 expression human non-parvicellular lung cancer A549 cells were suspended in Matrigel solution and 5,000,000 cells were subcutaneously transplanted into the chest of female BALB/c nude mouse (6 weeks of age). HER2 expression human kidney cancer ACHN cells (2,000,000 cells) were subcutaneously transplanted into the chest of nude mouse. HER2 high expression human gastric cancer cells 4-1ST was a in vivo subcultured tumor and seed tumor piece (ca. 50 mm³) was subcutaneously transplanted into the nude mouse. After the transplantation, the tumor diameter was measured (BT-474: day 29, A549, ACHN: day 10, 4-1ST: day 70) and 4 to 6 mice (varied depending on the tumor type) per group, uninformized with respect to tumor size, were used for the experiment.

The compound B4 was orally administered for 14 days (15 to 20 mg/kg/day, divided into two per day), or trastuzumab (Herceptin, product name) (10 mg/kg, twice a week) was intraperitoneally administered for 2 weeks. An increase in the tumor volume from that before administration to that on the next day of the final administration was compared to that of the control group and an anti-tumor effect was studied. In addition, the tumor volume was calculated from the following equation:

Formula: tumor volume=longer diameter×shorter diameter×shorter diameter×(1/2)

A ratio of the value obtained by subtracting a tumor volume on the day of the administration initiation from the tumor volume on the next day of the completion of the administration in the control group, and the value obtained by subtracting the tumor volume on the day of the administration initiation from the tumor volume on the next day of the completion of the administration was determined as a growth rate (T/C (%)). The results are shown in Table 6 (partial data cited from other Figure (BT-474, A549)).

The compound B4 showed an-antitumor activity on a breast cancer cell line and gastric cancer having a HercepTest™ score of 3+, and lung cancer and kidney cancer strain having a score of 1+, on which trastuzumab failed to show action. The compound B4 did not show antitumor activity on HER2 non-expression tumor.

The antitumor activity of compound B4 matched well with in vitro cell growth inhibitory activity and is effective against HER2 positive cases having poor prognosis, from among many patients with malignant solid tumors including intractable cancers such as non-parvicellular lung cancer and the like.

TABLE 6

| | | | Compound | | | |
|---|---|---|---|---|---|---|
| | | | B4 | | Trastuzmab | |
| | | | dose | | dose | |
| | | HER2 | (mg/kg/ | T/C | (mg/kg/ | T/C |
| Cell line | | score | day) | (%) | day) | (%) |
| BT-474 | breast | 3+ | 20 | −18 | | |
| 4-1ST | gastric | 3+ | 15 | 4 | | |
| A549 | lung | 1+ | 20 | 22 | 20 | 94 |
| ACHN | renal | 1+ | 20 | −16 | | |
| MDA-MB-231 | breast | 0 | 20 | 87 | | |

### Reference Example C11

### Selectivity of Compound B4 to HER2 Expression Cells (Growth Inhibitory Action in EGF Receptor Co-Expression Cells)

1) Cell growth inhibitory action; Each cell line was sown in a 24-well-multi-well plate (5×10⁴ cells/well) and cultured overnight in a carbon dioxide gas incubator (5% CO₂, 37° C.). The compound B4 was added and the cells were further cultured for 3 days in a carbon dioxide gas incubator, after which the cells were counted.

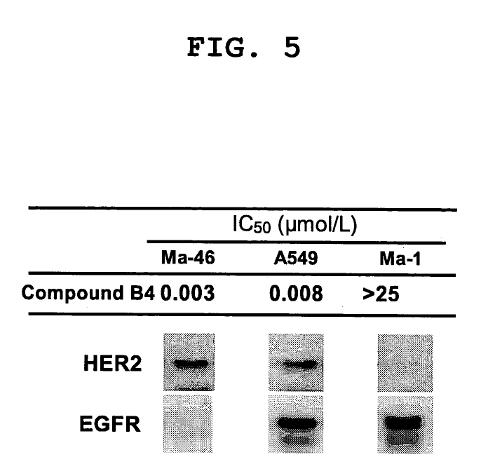

2) Receptor expression amount; Each cell line was sown in a 24-well multi-well plate and cultured overnight in a carbon dioxide gas incubator (5% CO₂, 37° C.). Then, the culture medium as removed and the cells were dissolved in an SDS sample buffer (200 μL). The cell lysate (8 μL) was separated by SDS-PAGE (7.5-15%) and after western blotting, HER2 and EGF receptor were detected by the ECL™ system and images were analyzed. The results are shown in FIG. 5.

The compound B4 showed a strong cell growth inhibitory action on HER2 expression cells (Ma-46), HER2/EGF-R co-expression cells (A549). The compound B4 did not show a cell growth inhibitory action on HER2 non-expression cells (Ma-1).

The EGF-R tyrosine kinase inhibitor (ZD-1839) shows a cell growth inhibitory action on EGF-R high expression cells, but the cell growth inhibitory action (IC₅₀) on HER2/EGF-R co-expression cells (A549) is 3 μmol/L and comparatively weak (Raben D et al. 11th NCI-EORTC-AACR symposium on new drug in cancer therapy, LB4 2000).

It has been reported that the HER2/EGF-R heterodimer plays a key role in the growth of cancer cells, and that HER2 tyrosine kinase activity is necessary, rather than EGF-R tyrosine kinase activity, for the activity expression of HER2/EGF-R heterodimer (Journal of Biological Chemistry, vol. 276, No. 18, pp. 15554 15560, 2001). The HER2 inhibitor is more effective than the EGF-R inhibitor in HER2/EGF-R co-expression cells.

### Reference Preparation Example 1

### (Amount per Tablet)

| | |
|---|---|
| (1) Compound obtained | 10.0 mg |
| in Reference Example B4 | |
| (2) Lactose | 60.0 mg |
| (3) Corn starch | 35.0 mg |
| (4) Gelatin | 3.0 mg |
| (5) Magnesium stearate | 2.0 mg |

A mixture of 10.0 mg of the compound obtained in Reference Example B4, 60.0 mg of lactose, and 35.0 mg of corn starch was granulated through a 1 mm-mesh sieve using 0.03 ml of a 10% by weight aqueous solution of gelatin (3.0 mg of gelatin), after which the granules were dried at 40° C. and filtered again. The granules obtained were mixed with 2.0 mg of magnesium stearate and compressed. The core tablets obtained were coated with a sugar coat comprising a suspension of sucrose, titanium dioxide, talc, and gum arabic and polished with beeswax to yield sugar-coated tablets.

### Reference Preparation Example 2

### (Amount per Tablet)

| | |
|---|---|
| (1) Compound obtained | 10.0 mg |
| in Reference Example B4 | |
| (2) Lactose | 70.0 mg |
| (3) Corn starch | 50.0 mg |
| (4) Soluble starch | 7.0 mg |
| (5) Magnesium stearate | 3.0 mg |

10.0 mg of the compound obtained in Reference Example B4 and 3.0 mg of magnesium stearate were granulated using 0.07 ml of an aqueous solution of soluble starch (7.0 mg of soluble starch), after which these granules were dried and mixed with 70.0 mg of lactose and 50.0 mg of corn starch. This mixture was compressed to yield tablets.

### INDUSTRIAL APPLICABILITY

Since the method of the present invention can block information signals of multimers of the epithelial growth factor receptor family by selectively inhibiting ErbB-2 (HER2), it is useful as a prophylactic or therapeutic method of cancer, a method of inhibiting cancer cell from acquiring drug resistance, a method of inhibiting metastasis of cancer cells to lymph node and the like.

1. A method for the prophylaxis or treatment of a cancer, which comprises blocking an information signal through a multimer of an epithelial growth factor receptor family by selectively inhibiting ErbB-2 (HER2).

2. The method of claim 1, wherein the information signal is blocked by inhibiting formation of a multimer between ErbB-2 (HER2) and the epithelial growth factor receptor family.

3. The method of claim 1 or 2, wherein the epithelial growth factor receptor family is EGFR (HER1), ErbB-2 (HER2), ErbB-3 (HER3) or ErbB-4 (HER4).

4. The method of claim 1, wherein the multimer of the epithelial growth factor receptor family is a heterodimer between (1) ErbB-2 (HER2) and (2) an epithelial growth factor receptor family selected from EGFR (HER1), ErbB-3 (HER3) and ErbB-4 (HER4).

5. The method of claim 1 to claim 4, wherein the cancer includes a cancer cell that tests HER2 positive by a HER2 diagnostic method.

6. The method of claim 1 to claim 5, wherein the ErbB-2 (HER2) is selectively inhibited by administering a HER2 tyrosine kinase inhibitor.

7. The method of claim 1 to claim 5, wherein the ErbB-2 (HER2) is selectively inhibited by administering an inhibitor of an extracellular moiety of ErbB-2 (HER2).

8. The method of claim 1 to claim 5, wherein the ErbB-2 (HER2) is selectively inhibited by administering a HER2 tyrosine kinase inhibitor and an anti-HER2 antibody in combination.

9. The method of claim 8, wherein the anti-HER2 antibody is Trastuzumab.

10. The method of claim 1 to claim 8, which is a prophylactic or therapeutic method of a drug resistant cancer.

11. The method of claim 1 to claim 8, which is a prophylactic or therapeutic method of a hormone independent cancer.

12. The method of claim 1 to claim 8, which is a prophylactic or therapeutic method of an anthracycline resistant cancer.

13. The method of claim 1 to claim 8, which is a prophylactic or therapeutic method of a taxon resistant cancer.

14. The method of claim 1 to claim 8, which is a prophylactic or therapeutic method of a platinum complex drug resistant cancer.

15. The method of claim 1 to claim 8, which delays or prevents cancer's becoming independent of hormone.

16. A method of inhibiting a cancer from acquiring drug resistance, which comprises selectively inhibiting ErbB-2 (HER2), thereby to block an information signal through a multimer of the epithelial growth factor receptor family.

17. A method of inhibiting metastasis of a cancer, which comprises selectively inhibiting ErbB-2 (HER2) to block an information signal through a multimer of the epithelial growth factor receptor family.

18. A method of inhibiting metastasis of a cancer to a lymph node, which comprises selectively inhibiting ErbB-2 (HER2) to block an information signal through a multimer of the epithelial growth factor receptor family.

19. A method for the prophylaxis or treatment of a cancer, which comprises inhibiting ErbB-2 (HER2) without causing downregulation of ErbB-2 (HER2) or going through an immune mechanism.

20. A method of regressing a cancer, which comprises selectively inhibiting ErbB-2 (HER2) to prevent involvement of the corresponding adaptor protein in ErbB-2 (HER2), thereby to block an information signal through a multimer of the epithelial growth factor receptor family.

21. A method of suppressing generation of an information signal through a HER2-HER3 dimer, which comprises inhibiting ErbB-2 (HER2), thereby to prevent involvement of the corresponding adaptor protein in ErbB-3 (HER3).

22. A method of delaying or prohibiting transition into a hormone independent cancer, which comprises administering a selective ErbB-2 (HER2) inhibitor to a patient with a hormone dependent cancer.

23. A method of treating a cancer, which comprises administering a selective ErbB-2 (HER2) inhibitor to a patient with a hormone independent cancer to make cancer cells hormone-dependent, and then administering a different anticancer agent and/or a hormone therapy agent to said patient.
